(19) United States
(12) Patent Application Publication
(10) Pub. No.: US 2026/0269032 A1
(43) Pub. Date: Sep. 10, 2026

(54) LANGUAGE MODEL-DRIVEN DENTAL DIAGNOSTICS SYSTEM

(71) Applicant: Align Technology, Inc., San Jose, CA (US)

(72) Inventors: Yury Slynko, Berlin (DE); Magdalena Blankenburg, Frankfurt am Main (DE); Eric Meyer, Morgan Hill, CA (US)

(21) Appl. No.: 19/558,294

(22) Filed: Mar. 5, 2026

Related U.S. Application Data
(60) Provisional application No. 63/768,029, filed on Mar. 6, 2025.
(60) Provisional application No. 63/885,106, filed on Sep. 19, 2025.

(51) Int. Cl. G16H 10/60; G16H 10/20; G16H 15/00; G16H 20/00; G16H 50/20; G16H 70/20

(52) U.S. Cl. CPC G16H 10/60; G16H 10/20; G16H 15/00; G16H 20/00; G16H 50/20; G16H 70/20

(57) ABSTRACT

Described herein are methods, systems, and devices for implementing a language model-driven dental diagnostics system. In at least one embodiment, a method includes: receiving, by the diagnostics system, a plurality of data sets corresponding to a patient; receiving, by the diagnostics system, data representative of text-based guidelines; processing the plurality of data sets and the text-based guidelines by applying a language model of the diagnostics system; and generating or updating a diagnostic record of the patient from output generated from the language model.

### CROSS-REFERENCE TO RELATED APPLICATIONS

This application claims the benefit of priority of U.S. Provisional Patent Application No. 63/768,029, filed Mar. 6, 2025, and U.S. Provisional Patent Application No. 63/885,106, filed Sep. 19, 2025, the disclosures of which are hereby incorporated by reference herein in their entireties.

### TECHNICAL FIELD

Embodiments of the present disclosure relate to the field of dental diagnostics and, in particular, to a system and method for improving the process of diagnosing dental conditions.

### BACKGROUND

For a typical dental practice, a patient visits the dentist twice a year for a cleaning and an examination. A dental office may or may not generate a set of dental x-ray images, intraoral scans, photos, videos, three-dimensional facial scans, or other types of scans of the patient during the patient visit. The dental hygienist additionally cleans the patient's teeth and notes any possible problem areas, which they convey to the dentist. The dentist then reviews the patient history, reviews the new images (if any such images were generated), and spends a few minutes examining the patient's teeth in a patient examination process. During the patient examination process, the dentist may follow a checklist of different areas to review. The examination can start with examining the patient's teeth for cavities, then reviewing existing restorations, then checking the patient's gums, then checking the patients'head, neck and mouth for pathologies or tumors, then checking the jaw joint, then checking the bite relationship and/or other orthodontic problems, and then checking any x-rays and/or other images of the patient. Based on this review, the dentist makes a determination as to whether there are any dental conditions that need to be dealt with immediately and whether there are any other dental conditions that are not urgent but that should be dealt with eventually and/or that should be monitored. The dentist then needs to explain the identified dental conditions to the patient, talk to the patient about potential treatments, and agree with the patient to make a decision on treatment for the patient's health. It can be challenging for the dentist to identify all problem dental conditions and convey the information about the dental conditions and their treatment options to the patient in the short amount of time that the dentist has allotted for that patient. The challenge is exacerbated by the fact that information about the patient's teeth can be fragmented and siloed, requiring the dentist to open and review multiple different applications and data sources to gain a full understanding of the patient's dental health and gum health. Sometimes the necessary information may exist at another provider's office including previous dentists and/or other dental specialists. This can lead to misdiagnosis of dental conditions and also increase the amount of time that the dentist must spend with each patient.

### SUMMARY

The following summary presents a simplified summary of various aspects of the present disclosure in order to provide a basic understanding of such aspects. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In one aspect of the present disclosure, a method comprises: receiving, by a diagnostics system, a plurality of data sets corresponding to a patient; receiving, by the diagnostics system, data representative of text-based guidelines; processing the plurality of data sets and the text-based guidelines by applying a language model of the diagnostics system, wherein the language model is configured to process one or more modalities; and generating or updating a diagnostic record of the patient from output generated from the language model.

In a further aspect of the present disclosure, a method comprises: receiving embeddings data generated from one or more sets of data corresponding to patient data; and providing the embeddings data as training data to an autoencoder to train the autoencoder to generate one or more of a treatment option, clinical indices, a diagnosis, a medical report, or model data.

In a further aspect of the present disclosure, a method comprises: receiving, by a diagnostics system, a plurality of data sets corresponding to a patient, wherein the plurality of data sets comprise data of different modalities; receiving, by the diagnostics system, data representative of text-based guidelines; processing, by the diagnostics system, each data set using a modality-specific machine learning model selected from a plurality of machine learning models to generate a representation of the data set, each modality-specific machine learning model being trained to process a data of a specific modality; aggregating the representations; providing the aggregated representations and the text-based guidelines as input to a language model of the diagnostics system; and generating or updating a diagnostic record of the patient based on output from the language model.

In a further aspect of the present disclosure, a method comprises: receiving embeddings data generated from one or more sets of data corresponding to patient data; and providing the embeddings data as training data to each of a plurality of modality-specific machine learning models.

In a further aspect of the present disclosure, a method comprises: providing, to a client device of a patient, access to a conversational agent, wherein the client device is to generate a graphical user interface (GUI) that allows the patient to interact with the conversational agent; implementing a chat conversation between the conversational agent and the patient, the chat conversation comprising a plurality of messages exchanged between the conversational agent and the patient, wherein the plurality of messages comprises: query messages transmitted to the client device derived from a medical questionnaire; and response messages received from the client device corresponding to responses of the patient to the query messages; processing the response messages received from the client device to generate a completed questionnaire; and storing the completed questionnaire in a dental record associated with the patient.

In a further aspect of the present disclosure, a method comprises: generating for display, by a client device, a graphical user interface (GUI), the GUI comprising a chat interface to facilitate a chat conversation between a user of the client device and a conversational agent; receiving a plurality of query messages from the conversational agent pertaining to a dental health questionnaire; displaying one or more proposed responses as selectable options; displaying, as a visual aid, one or more images or videos associated with content of one or more messages of the chat conversation; and transmitting responses entered by the user of the client device to the conversational agent.

In a further aspect of the present disclosure, a system comprises: a memory; and a processing device operatively connected to the memory, the processing device to perform the method of any of the preceding embodiments.

In a further aspect of the present disclosure, a computer readable medium comprises: instructions that, when executed by a processing device, cause the processing device to perform the method of any of the preceding embodiments.

### BRIEF DESCRIPTION OF THE DRAWINGS

The present disclosure is illustrated by way of example, and not by way of limitation, in the figures of the accompanying drawings.

FIG. 1A illustrates a user interface of a dental diagnostics hub, in accordance with embodiments of the present disclosure.

FIG. 1B illustrates a user interface of a dental diagnostics hub, showing a time-lapse feature, in accordance with embodiments of the present disclosure.

FIG. 1C illustrates a user interface of a dental diagnostics hub after diagnostics have been run on a patient's dental arches, in accordance with embodiments of the present disclosure.

FIG. 1D illustrates a user interface for navigating diagnostics results provided to a mobile device by a dental diagnostics hub, in accordance with embodiments of the present disclosure.

FIG. 1E illustrates a user interface of a dental diagnostics hub after diagnostics have been run on a patient's dental arches, in accordance with embodiments of the present disclosure.

FIG. 2 illustrates a user interface for a caries analysis of a dental diagnostics hub, in accordance with embodiments of the present disclosure.

FIG. 3 illustrates a user interface for a tooth wear analysis of a dental diagnostics hub, in accordance with embodiments of the present disclosure.

FIG. 4 illustrates a user interface for an occlusal contact analysis of a dental diagnostics hub, in accordance with embodiments of the present disclosure.

FIG. 5A illustrates a user interface for a malocclusion analysis of a dental diagnostics hub, in accordance with embodiments of the present disclosure.

FIG. 5B illustrates pre-treatment and post-treatment models of a dental arch in a user interface for an orthodontic treatment analysis of a dental diagnostics hub, in accordance with embodiments of the present disclosure.

FIG. 6A illustrates a user interface for a tooth stain analysis of a dental diagnostics hub, in accordance with embodiments of the present disclosure.

FIG. 6B illustrates a user interface for a post-bleaching tooth stain analysis of a dental diagnostics hub, in accordance with embodiments of the present disclosure.

FIG. 7 illustrates a patient dental score card of a dental diagnostics hub, in accordance with embodiments of the present disclosure.

FIG. 8 illustrates an intraoral scanning system, in accordance with embodiments of the present disclosure.

FIG. 9A illustrates a first portion of a flow diagram for a method of analyzing a patient's teeth and gums, in accordance with embodiments of the present disclosure

FIG. 9B illustrates a second portion of the flow diagram for a method of analyzing a patient's teeth and gums, in accordance with embodiments of the present disclosure.

FIG. 10 illustrates an exemplary data pipeline for implementing a diagnostics system, in accordance with embodiments of the present disclosure.

FIG. 11 illustrates a first variation of the data pipeline for which input to the diagnostics system is provided in the form of text data, in accordance with embodiments of the present disclosure.

FIG. 12 illustrates a second variation of the data pipeline for which input to the diagnostics system is provided in the form of text data and image data, in accordance with embodiments of the present disclosure.

FIG. 13 illustrates a third variation of the data pipeline for which input to the diagnostics system is provided in the form of embeddings, in accordance with embodiments of the present disclosure.

FIG. 14 is a flow diagram illustrating potential implementations of the diagnostics system for processing and generating outputs, in accordance with embodiments of the present disclosure.

FIG. 15 illustrates training of an autoencoder used in the exemplary data pipeline 1000, in accordance with embodiments of the present disclosure.

FIG. 16 illustrates a flow diagram for a method of generating or updating a diagnostic record of a patient, in accordance with embodiments of the present disclosure.

FIG. 17 illustrates an exemplary questionnaire system for providing an interactive conversational agent to converse with a patient, in accordance with embodiments of the present disclosure.

FIG. 18 illustrates an exemplary graphical user interface to provide a patient access to a conversational agent, in accordance with embodiments of the present disclosure.

FIG. 19A illustrates a flow diagram for a method of providing a patient access to a conversational agent, in accordance with embodiments of the present disclosure.

FIG. 19B illustrates a flow diagram for a method of providing a user interface on a client device for accessing a conversational agent, in accordance with embodiments of the present disclosure.

FIG. 20 illustrates a block diagram of an example computing device, in accordance with embodiments of the present disclosure.

### DETAILED DESCRIPTION

Described herein are embodiments of a dental diagnostics system. In at least one embodiment, a dentist or doctor (terms used interchangeably herein) and/or their dental staff may gather various information about a patient. Such information may include intraoral 3D scans of the patient's dental arches, x-rays of the patient's teeth (e.g., optionally including bitewing x-rays of the patient's teeth, panoramic x-rays of the patient's teeth, etc.), cone-beam computed tomography (CBCT) scans of the patient's jaw, infrared images of the patient's teeth, color 2D or 3D images of the patient's teeth and/or gums, videos of the patient's face, videos of the patient articulation in motion, biopsy information, malocclusion information, observation notes about the patient's teeth and/or gums, and so on. The intraoral scans may be generated by an intraoral scanner, and at least some of the other data may be generated by one or more devices other than intraoral scanners, such as mobile capture devices or specialized 3D/4D cameras. Additionally, different data may be gathered at different times. Each of the different data points may be useful for determining whether the patient has one or more types of dental conditions. In addition, text data (e.g., structured text data or unstructured text data) is also gathered. For example, the text data may be in the form of conversational language. The dental diagnostics system, in at least one embodiment, implements a large language model (LLM), such as a large multi-modal language model, to process, for example, the text data and potentially image data and generate one or more clinically-relevant outputs. In at least one embodiment, clinical data may be processed into text data prior to providing the clinical data to the dental diagnostics system. LLMs are multi-modal and may, in various embodiments, process data in addition to text data, such as images or video.

The dental diagnostics system analyzes one or more of the types of data that is available for a given patient, and uses that data to assess numerous different types of dental conditions for the patient and classify or rank identified dental conditions based on severity. The dental diagnostics system may implement a clinical protocol that is well-defined and is provided to the dental diagnostics system as guidance/instructions to formally follow. In at least one embodiment, the dental diagnostics hub includes a pre-established list of clinical areas for review and systematically processes the scan data and/or other data to populate the list with actual patient data and/or analysis of the actual patient data. The dental diagnostics system may be part of a dental diagnostics hub that provides a user interface that presents a unified view of each of the types of analyzed dental conditions, showing which of the types of dental conditions might be of concern and which of the types of dental conditions might not be of concern for the patient.

Traditionally, the various types of gathered information are fragmented such that each of the different types of information is stored in a separate system and accessed by a separate dentistry-related application. In order for a dentist to fully assess a patient's dental health, they generally would need to separately load and review each of the different types of data in each of the different dentistry-related applications specific to that type of data. The standard process for reviewing the data and making diagnoses involves numerous manual or automated steps on the part of the dentist, and requires significant effort and time on the part of the dentist. Accordingly, the standard process for a dentist to perform a full analysis of a patient's dental health is highly inefficient. Moreover, once such a full analysis is made, it is generally difficult for the dentist to present the diagnosis to the patient, which is again a highly manual process on the part of the dentist. What is lacking in traditional systems, and what is provided in embodiments described herein, is a way to quickly and consistently gather patient dental information in a systematic fashion and to store such information in a centralized repository. In at least one embodiment, some of the routine steps associated with gathering and/or reviewing dental information are automated to reduce error and increase efficiency.

The embodiments described herein address the limitations of traditional approaches by providing a holistic approach to dentistry by synthesizing data in a way to provide precise diagnoses from the level of individual teeth to the overall patient oral health status. Such diagnoses may take into account pertinent health and dental records, as well as the skillset and preferences of the dentist. The diagnostics system may also be able to follow a formal diagnostic protocol as defined by a specific academic or professional expert key opinion leader. By leveraging advanced analytical and reasoning capabilities, the diagnostics system described herein empowers dentists to deliver significantly improved diagnoses and, thus, treatments tailored to each patient's unique needs, enabling practitioners to identify subtle nuances and underlying issues to enhance the efficacy of intervention.

The dental diagnostics system is configured to process a variety of inputs. For example, the dental diagnostics system can import and analyze different types of patient inputs, including dental and medical patient questionnaires, photos, X-ray images, CBCT scans, 3D photos/models, videos, intraoral scans, intraoral pictures, patient history, doctor questionnaires, doctor notes, dental examination records (e.g., voice records, screenshots from existing patient management systems, etc.), periodontal examinations, articulation data, occlusion, bite data, dental and medical history forms, and other types of records. Input may also be processed by various language models for generating structured or unstructured text data and embeddings. The embodiments can utilize language models to contextually understand specific skills sets of a dentist and their preferences, and use those combined inputs and analyses to produce a comprehensive dental diagnosis within a logic frame of reasoning and with a specific and formal clinical protocol.

Outputs generated by the dental diagnostics system may be in the form of answers to questions provided by the doctor (e.g., in a chatbot interface), the generation of clinical records, diagnoses, and other clinically-relevant outputs, some of these being pre-defined in the clinical protocol. In at least one embodiment, the dental diagnostics system provides an interface through an interactive diagnostics hub to interact with the staff, the doctor and the patient. The language used to communicate with the user may be tailored to that individual's skill or knowledge level or native language. The dental diagnostics system is able to interact orally and/or visually with the users using AI vision, audio-based AI, deep learning, large language models, through an AI-based avatar, etc.

In at least one embodiment, the dental diagnostics system is configured to propose prioritized treatment strategies, goals and plans, including costs and duration of treatment, with reasoning derived from specific clinical protocols. The dental diagnostics system may be configured to export all of its diagnosis, treatment plans, reasoning into machine readable formats (e.g., JSON) for easy of integration into existing patient record systems.

In at least one embodiment, the dental diagnostics system brings together all of the disparate types of information associated with a patient's dental health. The dental diagnostics system further performs automated analysis for each of the different types of dental conditions. A summary result of the various automated analyses may then be shown together in a graphical user interface (GUI). The summary result of the various automated analyses may include a severity rating for each of the types of dental conditions. The summary result may identify those dental conditions having higher severity levels to call them to the attention of the dentist. The dentist may then select any of the types of dental conditions to cause the dental diagnostics system to provide more detailed information about the selected type of dental condition for the patient.

In at least one embodiment, the dental diagnostics system greatly increases the speed and efficiency of diagnosing dental conditions of patients. The dental diagnostics system enables a dentist to determine, at a single glance of the GUI for the dental diagnostics system, most or all of the dental conditions that might be of concern for a patient. It enables the dentist to easily and quickly prioritize dental conditions to be addressed. Additionally, the dental diagnostics system may compare different identified dental conditions to determine any correlations between different identified dental conditions. As a result, the dental diagnostics system may identify some dental conditions as symptoms of other underlying root cause dental conditions. For example, the dental diagnostics system may identify tooth crowding and caries formation that results from the tooth crowding, abfraction lesions, or dens invaginatus. Other examples can include identifying vertical root fracture or external cervical resorption.

Additionally, the dental diagnostics system in embodiments creates presentations of dental conditions, what will happen if those dental conditions are untreated, root causes of the patient's dental conditions, treatment plan options, and/or simulations of treatment results. Such presentations may be shown to the patient to educate the patient about the condition of their dentition and their options for treating the problems and/or leaving the problems untreated.

Further embodiments of the present disclosure relate to a conversational agent for collecting information to generate a completed patient questionnaire. Current approaches to collecting patient or consumer information, such as traditional paper or digital questionnaires, often present significant challenges. Many patients struggle to understand the questions or are unsure how to answer them, leading to incomplete or inaccurate responses. The sheer volume and complexity of these forms can be overwhelming, resulting in skipped questions, reduced engagement, and additional time consumption by the doctor or staff when they get an unfilled or partially filled questionnaire from a patient. This lack of clarity and guidance not only diminishes the quality of the collected data but also increases the administrative burden on healthcare providers, who must often follow up to clarify or complete missing information. Embodiments described herein that utilize a conversational agent offer a dynamic and interactive solution for gathering patient data. For example, by integrating virtual avatars (e.g., a computer-generated dentist) as part of a chat interview, adapting the types of questions based on previous answers, and extracting information from uploaded documents or media, the conversational agent provides a more intuitive and supportive experience. Moreover, such embodiments accommodate both voice and text responses, and are compatible with current chat platforms. The conversational agent may facilitate a chat conversation with a patient through their own personal device, which is implemented in a GUI. In at least one embodiment, the GUI includes additional data gathering features such as the ability to collect patient photos for dentofacial analysis, perform virtual smile designs, and conduct preliminary data analysis before a doctor's review.

As used herein, a “conversational agent” is software component executed by one or more computing devices that is configured to conduct an interactive exchange with a human user by receiving and generating messages in one or more modalities. A conversational agent may operate via text (e.g., chat), audio/voice (e.g., speech input and/or output), video (e.g., camera input and/or rendered video output), and/or an animated avatar (e.g., lip-synced, gesture-animated character), over synchronous or asynchronous communication channels. The agent maintains conversational context across turns, optionally across sessions, and may perform one or more tasks including collecting information, answering questions, providing guidance or education, controlling user interfaces, and initiating or orchestrating downstream workflows (e.g., data extraction, record updates, scheduling).

Advantages of the embodiments that utilize a conversational agent for generating a completed patient questionnaire include, but are not limited to: (1) enhanced patient comprehension: the conversational agent can dynamically adapt its language, tone, and explanations to match the patient's level of understanding; (2) increased engagement and completion rates: by presenting questions interactively and in manageable segments, the conversational agent minimizes patient overwhelm, encourages engagement, and significantly improves the rate of questionnaire completion compared to static and lengthy forms; (3) real-time clarification and education: the conversational agent can provide immediate clarifications, definitions, and illustrative examples in response to patient queries; (4) multimodal data collection: beyond text-based answers, the conversational agent can capture and integrate video, voice recordings, and uploaded documents, to generate a more robust and comprehensive dataset for clinical evaluation; (5) personalization and accessibility: the system can operate in the user's preferred language and accommodate various accessibility needs; (6) improved data quality: by guiding patients through the process and validating responses in real time, the conversational agent reduces the incidence of incomplete, inconsistent, or erroneous data; (7) seamless integration with clinical workflows: the collected data can be automatically structured and integrated into electronic health records or other dental software systems, streamlining the workflow for practitioners and reducing administrative burden; and (8) enhanced patient experience: the use of an avatar creates a familiar and reassuring interface to foster trust and comfort throughout the data collection process.

A dental practitioner (e.g., a dentist or dental technician) may use an intraoral scanner to perform an intraoral scan of a patient's oral cavity. An intraoral scan application running on a computing device operatively connected to the intraoral scanner may communicate with the scanner to effectuate intraoral scanning and receive intraoral scan data (also referred to as intraoral images and intraoral scans). A result of the intraoral scanning may be a sequence of intraoral scans that have been discretely generated (e.g., by pressing on a “generate scan” button of the scanner for each image) or automatically generated (e.g., by pressing a “start scanning” button and moving the intraoral scanner around the oral cavity while multiple intraoral scans are generated). An operator may start performing intraoral scanning at a first position in the oral cavity, and move the intraoral scanner within the oral cavity to various additional positions until intraoral scans have been generated for an entirety of one or more dental arches or until a particular dental site is fully scanned. In at least one embodiment recording of intraoral scans may start automatically as teeth are detected or insertion into the oral cavity is detected and may automatically be paused or stopped as removal of the intraoral scanner from the oral cavity is detected.

According to an example, a user (e.g., a dental practitioner) may subject a patient to intraoral scanning. In doing so, the user may apply the intraoral scanner to one or more patient intraoral locations. The scanning may be divided into one or more segments. As an example the segments may include a lower buccal region of the patient, a lower lingual region of the patient, a upper buccal region of the patient, an upper lingual region of the patient, one or more preparation teeth of the patient (e.g., teeth of the patient to which a dental device such as a crown or an orthodontic alignment device will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with scan being directed towards an interface area of the patient's upper and lower teeth). In one embodiment, the segments include an upper dental arch segment, a lower dental arch segment and a patient bite segment. Via such scanner application, the scanner may generate intraoral scan data. The computing device executing the intraoral scan application may receive and store the intraoral scan data. The intraoral scan data may include two-dimensional (2D) intraoral images (e.g., color 2D images), three-dimensional intraoral scans (e.g., intraoral images with depth information such as monochrome height maps), intraoral images generated using infrared or near-infrared (NIRI) light, and/or intraoral images generated using ultraviolet light. The 2D color images, 3D scans, NIRI and/or infrared images and/or ultraviolet images may be generated by an intraoral scanner capable of generating each of these types of intraoral scan data. Such intraoral scan data may be provided from the scanner to the computing device in the form of one or more points (e.g., one or more pixels and/or groups of pixels). For instance, the scanner may provide such intraoral scan data as one or more point clouds.

In at least one embodiment, intraoral scanning may be performed on a patient's oral cavity during a visitation of a dentist's office. The intraoral scanning may be performed, for example, as part of a semi-annual or annual dental health checkup. The intraoral scanning may be a full scan of the upper and lower dental arches, and may be performed in order to gather information for performing dental diagnostics. The dental information generated from the intraoral scanning may include 3D scan data, 2D color images, NIRI and/or infrared images, and/or ultraviolet images.

In addition to performing intraoral scanning, a dental practitioner may generate one or more other types of relevant dental health information, such as x-rays of the patient's teeth (e.g., optionally including bitewing x-rays of the patient's teeth, panoramic x-rays of the patient's teeth, etc.), cone-beam computed tomography (CBCT) scans of the patient's jaw, infrared images of the patient's teeth, color 2D images of the patient's teeth and/or gums not generated by an intraoral scanner (e.g., from photos taken by a camera), biopsy information, malocclusion information, observation notes about the patient's teeth and/or gums, and so on. Other types of relevant dental health information can include: results of comprehensive clinical examination including extraoral exam data (e.g., facial symmetry, lymph nodes, or TMJ) and/or intraoral exam data (e.g., teeth, gingiva, soft tissues, mucosa, tongue, floor of the mouth, or palate); results of periodontal evaluation (e.g., pocket depth probing and attachment loss measurement, bleeding on probing and gingival recession assessment, or periodontal charting; caries detection (e.g., visual-tactile examination, fiber-optic transillumination, or laser fluorescence devices such as DIAGNOdent); radiographic assessments (e.g., bitewing radiographs (interproximal caries detection), periapical radiographs (detailed view of individual teeth and roots), panoramic radiographs (overall jaw and dentition view), occlusal radiographs (assessment of anterior teeth and occlusal relationships), or cephalometric x-rays (orthodontic and skeletal analysis), or cone beam CT (3D imaging for complex cases)); endodontic testing, such as pulp vitality tests (e.g., cold, heat, electric pulp testing); occlusal analysis (e.g., articulator-based examination (occlusal discrepancies, bite registration), or digital occlusal analysis systems (e.g., T-Scan)); oral cancer screening (e.g., visual inspection and palpation for lesions or abnormal tissue changes); diagnostic casts and study models (e.g., impressions for model analysis, occlusion, and prosthodontic planning); TMJ evaluation (e.g., assessment of joint sounds, range of motion, and pain on movement); salivary and caries risk assessment (e.g., salivary flow and composition tests); cosmetic and aesthetic evaluations (e.g., smile design analysis and tooth color matching); and oral hygiene assessment (e.g., plaque, calculus, or biofilm evaluation); 3D facial scans (for symmetry and other facial/dental analysis); 3D video scans (for motion analysis, dynamic bite analysis, articulation analysis); and 2D articulation capture videos (for articulation/TMJ/functional analysis).

For example, in addition to a dental practitioner generating an intraoral scan of the oral cavity during an annual or semi-annual dentist appointment, the dental practitioner may additionally generate one or more x-rays of the patient's oral cavity during the dentist appointment. Additional types of dental information may also be gathered when the dentist deems it appropriate to generate such additional information. For example, the dentist may take biopsy samples and send them to a lab for testing and/or may generate a panoramic x-ray and/or a CBCT scan of the patient's oral cavity.

The intraoral scan application may generate a 3D model (e.g., a virtual 3D model) of the upper and/or lower dental arches of the patient from the intraoral scan data. To generate the 3D model(s) of the dental arches, the intraoral scan application may register and stitch together the intraoral scans generated from the intraoral scan session. In one embodiment, performing image registration includes capturing 3D data of various points of a surface in multiple intraoral scans, and registering the intraoral scans by computing transformations between the intraoral scans. The intraoral scans may then be integrated into a common reference frame by applying appropriate transformations to points of each registered intraoral scan.

In one embodiment, registration is performed for each pair of adjacent or overlapping intraoral scans. Registration algorithms may be carried out to register two adjacent intraoral scans for example, which essentially involves determination of the transformations which align one intraoral scan with the other. Registration may involve identifying multiple points in each intraoral scan (e.g., point clouds) of a pair of intraoral scans, surface fitting to the points of each intraoral scans, and using local searches around points to match points of the two adjacent intraoral scans. For example, the intraoral scan application may match points, edges, curvature features, spin-point features, etc. of one intraoral scan with the closest points, edges, curvature features, spin-point features, etc. interpolated on the surface of the other intraoral scan, and iteratively minimize the distance between matched points. Registration may be repeated for each adjacent and/or overlapping scans to obtain transformations (e.g., rotations around one to three axes and translations within one to three planes) to a common reference frame. Using the determined transformations, the intraoral scan application may integrate the multiple intraoral scans into a first 3D model of the lower dental arch and a second 3D model of the upper dental arch.

The intraoral scan data may further include one or more intraoral scans showing a relationship of the upper dental arch to the lower dental arch. These intraoral scans may be usable to determine a patient bite and/or to determine occlusal contact information for the patient. The patient bite may include determined relationships between teeth in the upper dental arch and teeth in the lower dental arch.

The intraoral scan application or another application may further register data from one or more other imaging modalities to the 3D model generated from the intraoral scan data. For example, processing logic may register x-ray images, CBCT scan data, ultrasound images, panoramic x-ray images, 2D color images, NIRI images, articulation data, various other video data or 3D scan data, and so on to the 3D model. Each of the different imaging modalities may contribute different information about the patient's dentition. For example, NIRI images and x-ray images may identify caries and color images may be used to add accurate color data to the 3D model, which is usable to determine tooth staining. The registered intraoral data from the multiple imaging modalities may be presented together in the 3D model and/or side-by-side with one or more imaging modalities shown that reflect a zoomed in and/or highlighted section and/or orientation of the 3D model. The data from different imaging modalities may be provided as different layers, where each layer may be for a particular imaging modality. This may enable a doctor to turn on or off specific layers to visualize the dental arch with or without information from those particular imaging modalities.

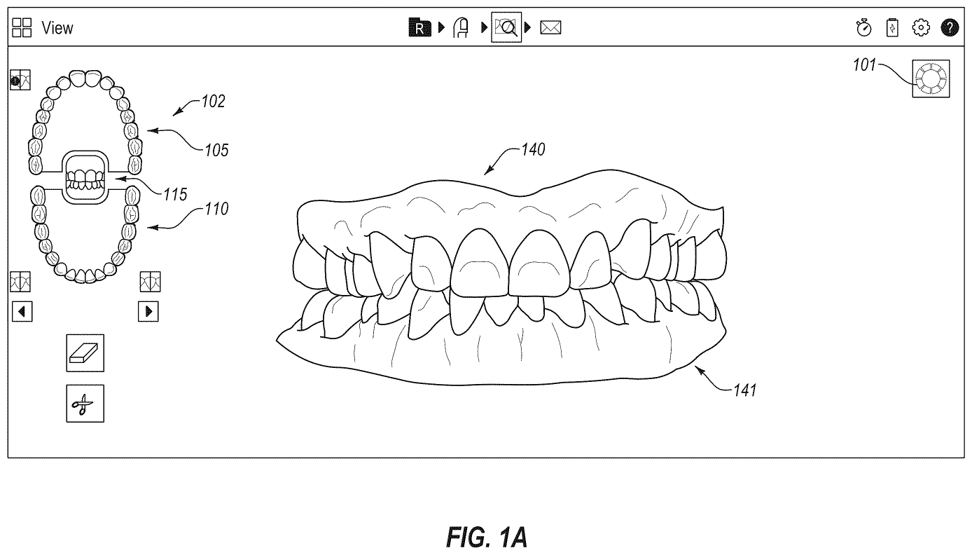

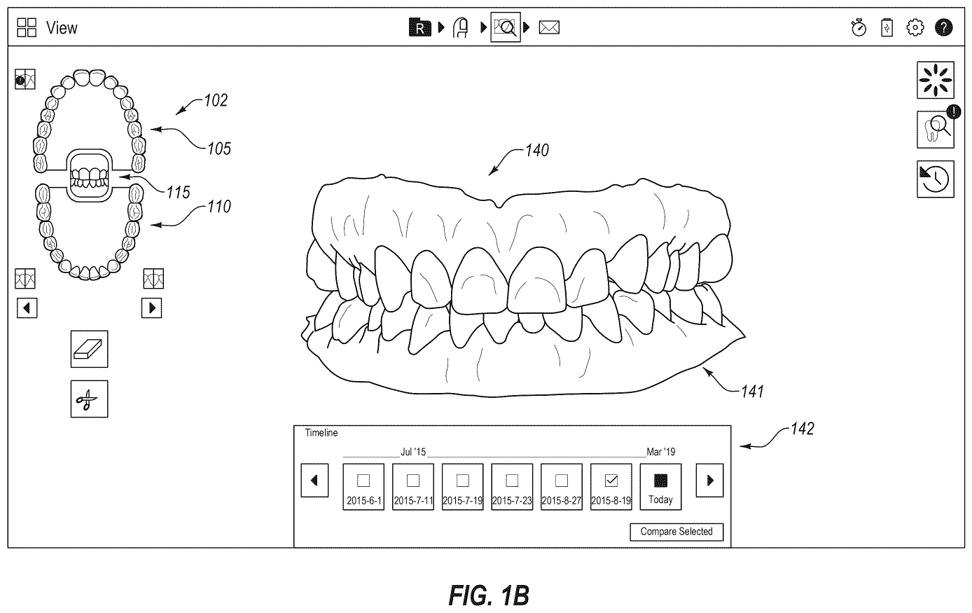

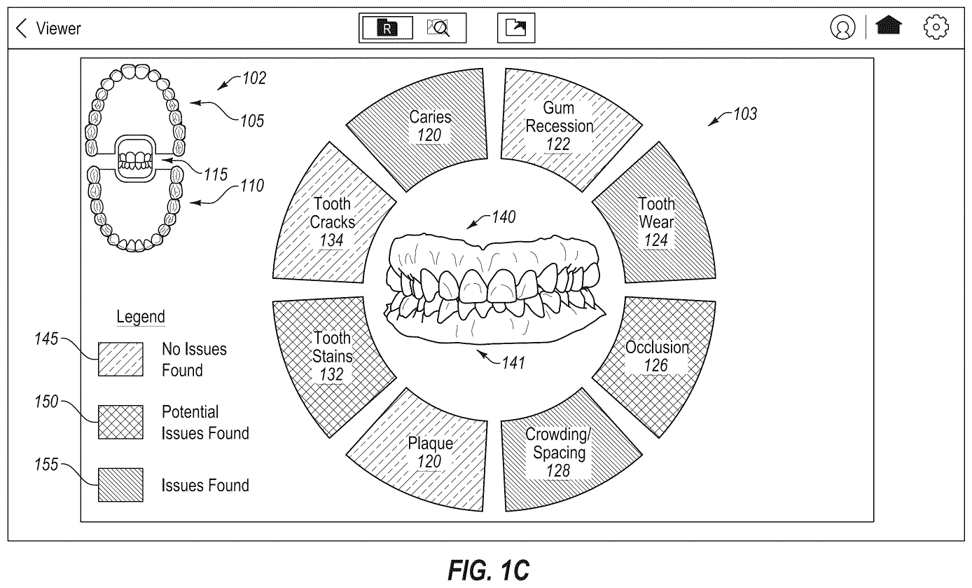

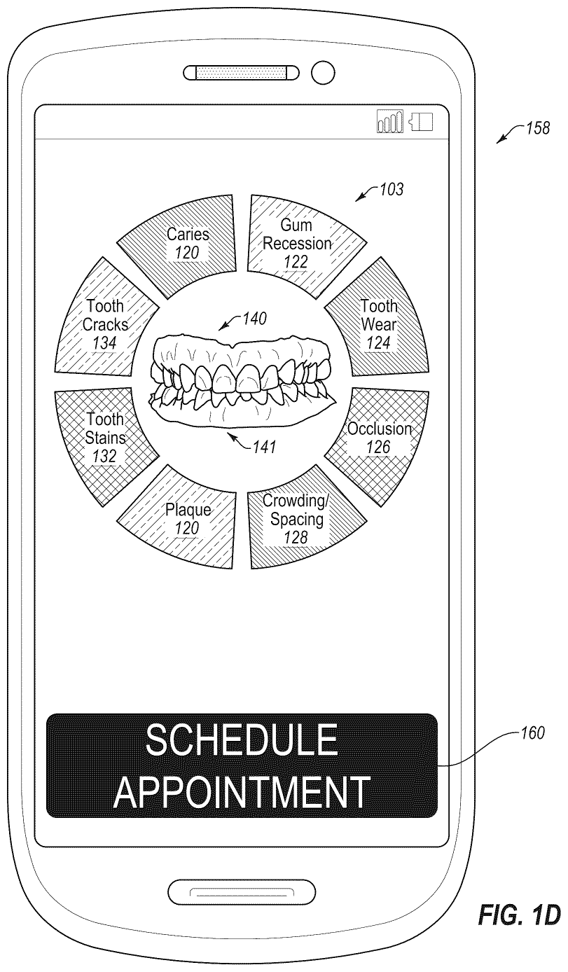

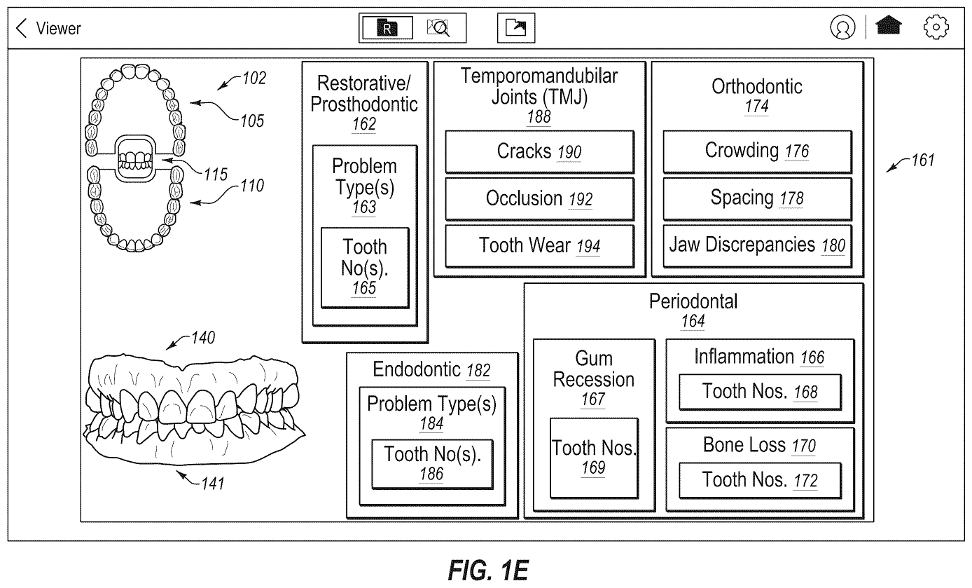

FIG. 1A illustrates a user interface of a dental diagnostics hub, in accordance with embodiments of the present disclosure. A 3D model of a patient's upper dental arch 140 and a 3D model of the patient's lower dental arch 141 may be generated by an intraoral scan application and input into the dental diagnostics hub. Additionally, bite data showing the relationship of the upper dental arch 140 and the lower dental arch 141 may be input into the dental diagnostics hub. The bite relationship data may also include how the upper and lower jaw dynamically relate to each other in functional motions and not just in a static relationship relative to each other. This can be useful for diagnostic problems related to the jaw joint (e.g., temporomandibular (TMJ) disorders). Additionally, intraoral scans may have been generated of one or more preparation tooth of the patient, which may also be input to the dental diagnostics hub. The dental diagnostics hub may then present a view of the upper dental arch 140, the lower dental arch 141, the preparation teeth, and/or the relative positions of the upper and lower dental arches 140, 141 in the user interface of the dental diagnostics hub.

Via the user interface of the dental diagnostics hub, a practitioner may view one or more of the upper dental arch 140, the lower dental arch 141, a particular preparation tooth and/or the patient bite, each of which may be considered a separate scan segment or mode. The practitioner may select one or multiple scan segments to view via a scan segment selector 102. In one embodiment, as shown, the scan segment selector 102 may include an upper dental arch segment selection 105, a lower dental arch segment selection 110 and a bite segment selection 115. As illustrated, the upper dental arch segment selection 105 and the lower dental arch segment selection 110 are active, causing the 3D model of the upper dental arch 140 and the 3D model of the lower dental arch 141 to be shown. A practitioner may rotate the 3D models and/or change a zoom setting for a view of the 3D models using the GUI.

The GUI of the dental diagnostics hub may further include a diagnostics command 101. Selection of the diagnostics command 101 may cause the dental diagnostics hub to perform one or multiple different analyses of the patient's dental arches 140, 141 and/or bite. The analyses may include an analysis for identifying tooth cracks, an analysis for identifying gum recession, an analysis for identifying tooth wear, an analysis of the patient's occlusal contacts, an analysis for identifying crowding of teeth (and/or spacing of teeth) and/or other malocclusions, an analysis for identifying plaque, an analysis for identifying tooth stains, an analysis for identifying caries, and/or other analyses of the patient's dentition. Once the analyses are complete, a dental diagnostics summary may be generated and shown in the GUI of the dental diagnostics hub, as shown in FIG. 1C.

Some of the analyses that are performed to assess the patient's dental health are dental condition progression analyses that compare dental conditions of the patient at multiple different points in time. For example, one caries assessment analysis may include comparing caries at a first point in time and a second point in time to determine a change in severity of the caries between the two points in time, if any. Other time-based comparative analyses that may be performed include a time-based comparison of gum recession, a time-based comparison of tooth wear, a time-based comparison of tooth movement, a time-based comparison of tooth staining, and so on. In at least one embodiment processing logic automatically selects data collected at different points in time to perform such time-based analyses. Alternatively, a user may manually select data from one or more points in time to use for performing such time-based analyses.

FIG. 1B illustrates a user interface of a dental diagnostics hub, showing a time-lapse feature 142, in accordance with embodiments of the present disclosure. In one embodiment, the time-lapse feature is launched automatically when a user selects the diagnostics command 101 to provide the user an option to select which dental information from which points in time to use for the analyses to be performed. In one embodiment, the time lapse feature 142 shows each of the different points in time (i.e., different times stamps) at which dental information was collected along a time line. The dental information that may be selected may include at a minimum intraoral scan data (e.g., 3D models generated based on one or more intraoral scanning sessions). The dental information that may be selected may further include x-rays generated at various points in time, CBCT scan data generated at various points in time, and/or other dental information generated at various points in time. Via the time lapse feature 142, a user may select one or more past data points (e.g., for previously generated 3D models of dental arches) and/or one or more current data points or most recent data points (e.g., for a current 3D model of the dental arches). The selected data points may then be used to perform one or more time-based analyses of the patient's dentition.

In at least one embodiment, the time-based analyses of the patient's dentition compare 3D models and/or one or more dental conditions of the patient over time, and identify dental conditions and/or determine a rate of progression of the one or more dental conditions based on the comparison. For example, 3D models of the dental arches from different points in time may be compared to one another to determine rates of progression of tooth wear, caries development, gum recession, gum swelling, malocclusions, and so on. The rates of progression may be compared to rate of progression thresholds. The rate of progression thresholds may be set by a doctor or may be set to defaults. Amount of change for dental conditions may also be determined, and may be compared to amount of change thresholds. Those dental conditions for which the rate of progression meets or exceeds a rate of progression threshold for that dental condition and/or for which amount of change meets or exceeds an amount of change threshold may be identified as dental conditions that are of clinical significance and/or dental conditions for which issues or problems have been identified.

The time-based analyses may project detected rates of progression or rates of change of one or more dental conditions into the future to predict severity levels of the dental conditions at future points in time. In at least one embodiment, progression of one or more dental conditions may be projected into the future, and the predicted dental condition at each projected point in time may be compared to one or more criteria (e.g., such as a severity threshold). The one or more criteria may be default criteria and/or may be criteria set by a doctor (e.g., a user of the dental diagnostics hub). The criteria may also be set by aggregated data, either within the same practice or through a network of practices of similar patient traits. When the one or more criteria are satisfied, that indicates that a dental condition of clinical significance is identified. The future point in time at which a projected dental condition will satisfy the one or more criteria (e.g., pass the severity threshold) may be noted and added to the patient's record in embodiments. In at least one embodiment, if the future point in time at which a projected dental condition will satisfy the one or more criteria for that dental condition is within a threshold amount of time from a current date, then the dental condition may be identified as of clinical importance or of potential clinical importance.

FIG. 1C illustrates a user interface of a dental diagnostics hub showing a dental diagnostics summary 103 after diagnostics have been run on a patient's dental arches, in accordance with embodiments of the present disclosure. In at least one embodiment, the dental diagnostics summary 103 includes the scan segment selector 102 including upper dental arch segment selection 105, lower dental arch segment selection 110 and/or bite segment selection 115. In at least one embodiment, the dental diagnostics summary 103 further includes views of the selected dental segments or modes (e.g., of the 3D model of the upper dental arch 140 and the 3D model of the lower dental arch 141 of the patient).

The dental diagnostics summary 103 provides a single view showing multiple different types of possible dental conditions and assessments as to the presence and/or severity of each of the types of dental conditions. In one embodiment, the various dental conditions are assigned one of three severity levels, including “no issues found” 145, “potential issues found” 150 and “issues found” 155. Each of the dental conditions may be coded or labeled with the severity ranking determined for that type of dental condition. In one embodiment, the dental conditions are color coded to graphically show severity levels. For example, those dental conditions for which issues were found may be coded red, those dental conditions for which potential issues were found may be coded yellow, and those dental conditions for which no issues were found may be coded green. Many other coding schemes are also possible. In one embodiment, each of the dental conditions is assigned a numeric severity level. For example, on a scale of 1 to 100, each dental condition may be assigned a severity level between 1 and 100 to indicate the severity level of that dental condition. Those dental conditions with a severity level that is below a first threshold severity level may be identified as dental conditions for which no issues were found. Those dental conditions for which the severity level is above the first threshold severity level but below a second threshold severity level may be identified as dental conditions for which potential issues were found. Those dental conditions for which the severity level is above the second severity level threshold may be identified as dental conditions for which issues were found. In at least one embodiment, different severity level thresholds may be set for each of the different dental conditions. Alternatively, the severity levels of the different dental conditions may be normalized across the multiple types of dental conditions and the same severity level thresholds may be used for multiple dental conditions. In at least one embodiment dental conditions are ranked based on their severity levels and/or based on the different between their severity levels and the associated severity level threshold for the dental conditions.

In at least one embodiment, a doctor may set severity level thresholds for one or more of the dental conditions. Severity level thresholds that a doctor may set may be point-in-time severity level thresholds for point-in-time severity levels of dental conditions determined based on data from a single point in time. Additionally, or alternatively, severity level thresholds that a doctor may set may be time-dependent thresholds, such as amount of change thresholds and rate of change thresholds. Alerts may be set to remind the doctor when the threshold level is approaching specific criteria.

Absent such selected severity level thresholds, default severity level thresholds may be automatically set for one or more of the dental conditions. In an example, a doctor may set a caries size threshold, and any detected caries that have a size that meets or exceeds the set caries size threshold may be identified as a found issue. In another example, a doctor may set a gum recession amount threshold, and any identified gum recession that has a value that meets or exceeds the gum recession amount threshold may be identified as a found issue. A doctor may also set rate of change thresholds for one or more dental conditions and/or such rate of change thresholds may be automatically set to default values. For example, if the rate of change of tooth wear exceeds a tooth wear rate of change threshold, then the patient may be identified as having an identified tooth wear issue. A doctor may also set an amount of change threshold. If a detected amount of change is greater than the set amount of change threshold for a dental condition, then the doctor may be alerted. Multiple different units may be used to set severity level thresholds, such as units of distance (e.g., microns, millimeters, fractions of an inch, etc.), units of size (e.g., microns, millimeters, fractions of an inch, etc.), units of rates of change (e.g., microns/month, millimeters per year, etc.), units of luminance, units of volume (e.g., mm³), units of area (e.g., mm²), ratios, percentages (e.g., percentage of change), and so on.

In at least one embodiment, severity level thresholds may depend at least in part on a location of an identified dental condition. For example, different caries severity thresholds may be set for different locations. Caries that are close to dentin may be more urgent because they are more likely to cause pain and/or to require a root canal than caries that are far from dentin. Accordingly, caries that are close to dentin may have a lower threshold than caries that are far from dentin, for example. In at least one embodiment, the distance between a caries and a patient's dentin may be determined based on x-ray data, a CBCT scan and/or NIRI imaging of the intraoral cavity.

In one embodiment, the different types of dental conditions for which analyses are performed and that are included in the dental diagnostics summary 103 include tooth cracks, gum recession, tooth wear, occlusal contacts, crowding and/or spacing of teeth and/or other malocclusions, plaque, tooth stains, and caries. Additional, fewer and/or alternative dental conditions may also be analyzed and reported in the dental diagnostics summary 103. In at least one embodiment, multiple different types of analyses are performed to determine presence and/or severity of one or more of the dental conditions. One type of analysis that may be performed is a point-in-time analysis that identifies the presence and/or severity levels of one or more dental conditions at a particular point-in-time based on data generated at that point-in-time. For example, a single 3D model of a dental arch may be analyzed to determine whether, at a particular point-in-time, a patient's dental arch included any caries, gum recession, tooth wear, problem occlusion contacts, crowding, spacing or tooth gaps, plaque, tooth stains, and/or tooth cracks. Another type of analysis that may be performed is a time-based analysis that compares dental conditions at two or more points in time to determine changes in the dental conditions, progression of the dental conditions and/or rates of change of the dental conditions, as discussed with reference to FIG. 1B. For example, in embodiments a comparative analysis is performed to determine differences between 3D models of dental arches taken at different points in time. The differences may be measured to determine an amount of change, and the amount of change together with the times at which the intraoral scans that were used to generate the 3D models were taken may be used to determine a rate of change. This technique may be used, for example, to identify an amount of change and/or a rate of change for tooth wear, staining, plaque, crowding, spacing, gum recession, caries development, tooth cracks, and so on.

In at least one embodiment, one or more trained models are used to perform at least some of the one or more dental condition analyses. The trained models may include physics models and/or machine learning models, for example. In one embodiment, a single model may be used to perform multiple different analyses (e.g., to identify any combination of tooth cracks, gum recession, tooth wear, occlusal contacts, crowding and/or spacing of teeth and/or other malocclusions, plaque, tooth stains, and/or caries). Additionally, or alternatively, different models may be used to identify different dental conditions. For example, a first model may be used to identify tooth cracks, a second model may be used to identify tooth wear, a third model may be used to identify gum recession, a fourth model may be used to identify problem occlusal contacts, a fifth model may be used to identify crowding and/or spacing of teeth and/or other malocclusions, a sixth model may be used to identify plaque, a sixth model may be used to identify tooth stains, and/or a seventh model may be used to identify caries.

In one embodiment, intraoral data from one or more points in time are input into one or more trained machine learning models that have been trained to receive the intraoral data as an input and to output classifications of one or more types of dental conditions. In one embodiment, the trained machine learning model(s) is trained to identify areas of interest (AOIs) from the input intraoral data and to classify the AOIs based on dental conditions. The AOIs may be or include regions associated with particular dental conditions. The regions may include nearby or adjacent pixels or points that satisfy some criteria, for example. The intraoral data that is input into the one or more trained machine learning model may include three-dimensional (3D) data and/or two-dimensional (2D) data. The intraoral data may include, for example, one or more 3D models of a dental arch, one or more projections of one or more 3D models of a dental arch onto one or more planes (optionally comprising height maps), one or more x-rays of teeth, one or more CBCT scans, a panoramic x-ray, near-infrared and/or infrared imaging data, color image(s), ultraviolet imaging data, intraoral scans, and so on. If data from multiple imaging modalities are used (e.g., 3D scan data, color images, and NIRI imaging data), then the data may be registered and/or stitched together so that the data is in a common reference frame and objects in the data are correctly positioned and oriented relative to objects in other data. One or more feature vectors may be input into the trained model, where the feature vectors include multiple channels of information for each point or pixel of an image. The multiple channels of information may include color channel information from a color image, depth channel information from intraoral scan data, a 3D model or a projected 3D model, intensity channel information from an x-ray image, and so on.

The trained machine learning model(s) may output a probability map, where each point in the probability map corresponds to a point in the intraoral data (e.g., a pixel in an intraoral image or point on a 3D surface) and indicates probabilities that the point represents one or more dental classes. In one embodiment, a single model outputs probabilities associated with multiple different types of dental classes, which includes one or more dental condition classes. In an example, a trained machine learning model may output a probability map with probability values for a teeth dental class and a gums dental class. The probability map may further include probability values for tooth cracks, gum recession, tooth wear, occlusal contacts, crowding and/or spacing of teeth and/or other malocclusions, plaque, tooth stains, healthy area (e.g., healthy tooth and/or healthy gum) and/or caries. In the case of a single machine learning model that can identify each of tooth cracks, gum recession, tooth wear, occlusal contacts, crowding and/or spacing of teeth and/or other malocclusions, plaque, tooth stains, and caries, eleven valued labels may be generated for each pixel, one for each of teeth, gums, healthy area, tooth cracks, gum recession, tooth wear, occlusal contacts, crowding and/or spacing of teeth and/or other malocclusions, plaque, tooth stains, and caries. The corresponding predictions have a probability nature: for each pixel there are multiple numbers that may sum up to 1.0 and can be interpreted as probabilities of the pixel to correspond to these classes. In one embodiment, the first two values for teeth and gums sum up to 1.0 and the remaining values for healthy area, tooth cracks, gum recession, tooth wear, occlusal contacts, crowding and/or spacing of teeth and/or other malocclusions, plaque, tooth stains, and/or caries sum up to 1.0.

In some instances, multiple machine learning models are used, where each machine learning model identifies a subset of the possible dental conditions. For example, a first trained machine learning model may be trained to output a probability map with three values, one each for healthy teeth, gums, and caries. Alternatively, the first trained machine learning model may be trained to output a probability map with two values, one each for healthy teeth and caries. A second trained machine learning model may be trained to output a probability map with three values (one each for healthy teeth, gums and tooth cracks) or two values (one each for healthy teeth and tooth cracks). One or more additional trained machine learning models may each be trained to output probability maps associated with identifying specific types of dental conditions.

In case of an ML model trained to identify three classes, it is convenient to store such predictions of dental classes in an RGB format. For example, a first value for a first dental class may be stored as a red intensity value, a second value for a second dental class may be stored as a green intensity value, and a third value for a third dental class may be stored as a blue intensity value. This may make visualization of the probability map very easy. Usually, there is no need in high precision and chars can be used instead of floats-that is 256 possible values for every channel of the pixel. Further optimization can be done in order to reduce the size and improve performance (e.g., use 16 values quantization instead of 256 values).

The output of the one or more trained machine learning models may be used to update one or more versions of the 3D model of the patient's upper and/or lower dental arches. In one embodiment, a different layer is generated for each dental condition class. A layer may be turned on to graphically illustrate areas of interest on the upper and/or lower dental arch that has been identified or flagged as having a particular dental condition.

If the probability maps were generated for one or more input 2D images (e.g., such as height maps in which pixel intensity represents height or depth), the probability maps output by the ML model(s) may be projected onto the points in the virtual 3D model. Accordingly, each point in the virtual 3D model may include probability information from probability maps of one or multiple different intraoral images that map to that point. In one embodiment, the probability information from the probability map is projected onto the 3D model as a texture. The updated 3D model may then include, for one or more points, vertexes or voxels of the 3D model (e.g., vertexes on a 3D mesh that represents the surface of the 3D model), multiple sets of probabilities, where different sets of probabilities associated with probability maps generated for different input images or other intraoral data may have different probability values.

Processing logic may modify the virtual 3D model by determining, for each point in the virtual 3D model, one or more dental class for that point. This may include using a voting function to determine a dental class for each point. For example, each set of probability values from an intraoral image may indicate a particular dental class. Processing logic may determine the number of votes for each dental class for a point, and may then classify the point as having a dental class that receives the most votes. In at least one embodiment points may be associated with multiple classes of dental conditions.

In at least one embodiment, image processing and/or 3D data processing may be performed on 3D models of dental arches generated from intraoral scans and/or on the output of one or more trained models. Such image processing and/or 3D data processing may be performed using one or more algorithms, which may be generic to multiple types of dental conditions or may be specific to particular dental conditions. For example, a trained model may identify regions on a 3D model of a dental arch that include caries, and image processing may be performed to assess the size and/or severity of the identified caries. The image processing may include performing automated measurements such as size measurements, distance measurements, amount of change measurements, rate of change measurements, ratios, percentages, and so on. Accordingly, the image processing and/or 3D data processing may be performed to determine severity levels of dental conditions identified by the trained model(s). Alternatively, the trained models may be trained both to classify regions as caries and to identify a severity and/or size of the caries.

The one or more trained machine learning models that are used to identify, classify and/or determine a severity level for dental conditions may be neural networks such as deep neural networks or convolutional neural networks. Such machine learning models may be trained using supervised training in embodiments.

Artificial neural networks (e.g., deep neural networks and convolutional neural networks) generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, lips, gums, etc.); and the fourth layer may recognize that the image contains a face or define a bounding box around teeth in the image. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

In one embodiment, a U-net architecture is used. A U-net is a type of deep neural network that combines an encoder and decoder together, with appropriate concatenations between them, to capture both local and global features. The encoder is a series of convolutional layers that increase the number of channels while reducing the height and width when processing from inputs to outputs, while the decoder increases the height and width and reduces the number of channels. Layers from the encoder with the same image height and width may be concatenated with outputs from the decoder. Any or all of the convolutional layers from encoder and decoder may use traditional or depth-wise separable convolutions.

In one embodiment, the machine learning model is a recurrent neural network (RNN). An RNN is a type of neural network that includes a memory to enable the neural network to capture temporal dependencies. An RNN is able to learn input-output mappings that depend on both a current input and past inputs. The RNN will address past and future intraoral data (e.g., intraoral scans taken at different times) and make predictions based on information that spans multiple time periods and/or patient visits. RNNs may be trained using a training dataset to generate a fixed number of outputs. One type of RNN that may be used is a long short term memory (LSTM) neural network.

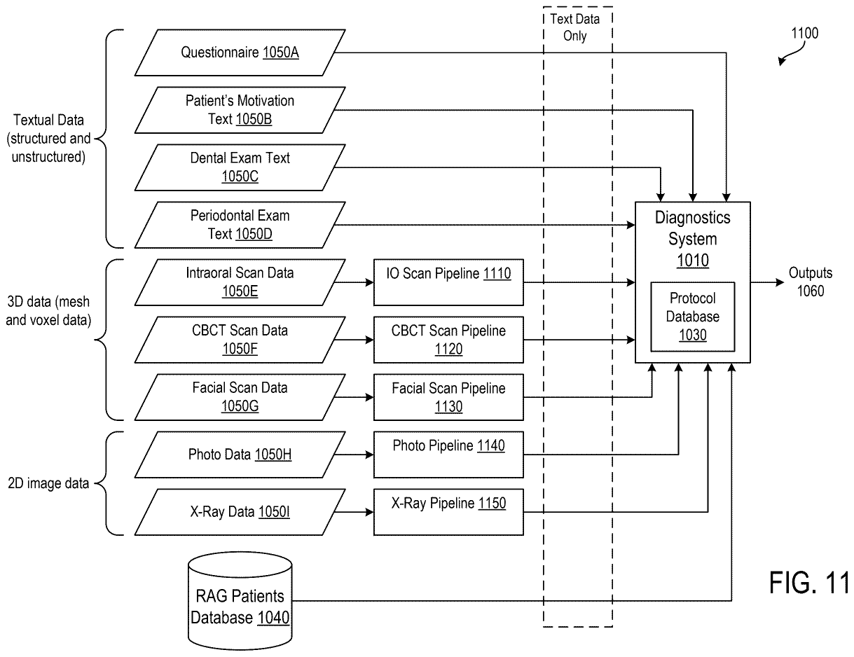

A common architecture for such tasks is LSTM (Long Short Term Memory). Unfortunately, LSTM is not well suited for images since it does not capture spatial information as well as convolutional networks do. For this purpose, one can utilize ConvLSTM—a variant of LSTM containing a convolution operation inside the LSTM cell. ConvLSTM is a variant of LSTM (Long Short-Term Memory) containing a convolution operation inside the LSTM cell. ConvLSTM replaces matrix multiplication with a convolution operation at each gate in the LSTM cell. By doing so, it captures underlying spatial features by convolution operations in multiple-dimensional data. The main difference between ConvLSTM and LSTM is the number of input dimensions. As LSTM input data is one-dimensional, it is not suitable for spatial sequence data such as video, satellite, radar image data set. ConvLSTM is designed for 3-D data as its input. In one embodiment, a CNN-LSTM machine learning model is used. A CNN-LSTM is an integration of a CNN (Convolutional layers) with an LSTM. First, the CNN part of the model processes the data and a one-dimensional result feeds an LSTM model. The network architecture for excess material removal may look as is shown in FIGS. 11A-B in one embodiment, which includes a ConvLSTM machine learning model.

In one embodiment, a class of machine learning model called a MobileNet is used. A MobileNet is an efficient machine learning model based on a streamlined architecture that uses depth-wise separable convolutions to build light weight deep neural networks. MobileNets may be convolutional neural networks (CNNs) that may perform convolutions in both the spatial and channel domains. A MobileNet may include a stack of separable convolution modules that are composed of depthwise convolution and pointwise convolution (conv 1×1). The separable convolution independently performs convolution in the spatial and channel domains. This factorization of convolution may significantly reduce computational cost from HWNK2M to HWNK2 (depthwise) plus HWNM (conv 1×1), HWN(K2+M) in total, where N denotes the number of input channels, K2 denotes the size of convolutional kernel, M denotes the number of output channels, and H×W denotes the spatial size of the output feature map. This may reduce a bottleneck of computational cost to conv 1×1.

In one embodiment, a generative adversarial network (GAN) is used. A GAN is a class of artificial intelligence system that uses two artificial neural networks contesting with each other in a zero-sum game framework. The GAN includes a first artificial neural network that generates candidates and a second artificial neural network that evaluates the generated candidates. The GAN learns to map from a latent space to a particular data distribution of interest (a data distribution of changes to input images that are indistinguishable from photographs to the human eye), while the discriminative network discriminates between instances from a training dataset and candidates produced by the generator. The generative network's training objective is to increase the error rate of the discriminative network (e.g., to fool the discriminator network by producing novel synthesized instances that appear to have come from the training dataset). The generative network and the discriminator network are co-trained, and the generative network learns to generate images that are increasingly more difficult for the discriminative network to distinguish from real images (from the training dataset) while the discriminative network at the same time learns to be better able to distinguish between synthesized images and images from the training dataset. The two networks of the GAN are trained once they reach equilibrium. The GAN may include a generator network that generates artificial intraoral images and a discriminator network that segments the artificial intraoral images. In at least one embodiment, the discriminator network may be a MobileNet.

In one embodiment, the machine learning model is a conditional generative adversarial (cGAN) network, such as pix2pix. These networks not only learn the mapping from input image to output image, but also learn a loss function to train this mapping. GANs are generative models that learn a mapping from random noise vector z to output image y, G: z→y. In contrast, conditional GANs learn a mapping from observed image x and random noise vector z, to y, G: {x, z}→y. The generator G is trained to produce outputs that cannot be distinguished from “real” images by an adversarially trained discriminator, D, which is trained to do as well as possible at detecting the generator's “fakes”. The generator may include a U-net or encoder-decoder architecture in embodiments. The discriminator may include a MobileNet architecture in embodiments. An example of a cGAN machine learning architecture that may be used is the pix2pix architecture described in Isola, Phillip, et al. “Image-to-image translation with conditional adversarial networks.” arXiv preprint (2017).

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

To train the one or more machine learning models, a training dataset (or multiple training datasets, one for each of the machine learning models to be trained) containing hundreds, thousands, tens of thousands, hundreds of thousands or more images should be used to form a training dataset. In at least one embodiment, up to millions of cases of patient dentition that include one or more labeled dental conditions such as cracked teeth, tooth wear, caries, gum recession, gum swelling, tooth stains, healthy teeth, healthy gums, and so on are used, where each case may include a final virtual 3D model of a dental arch (or other dental site such as a portion of a dental arch). The machine learning models may be trained to automatically classify and/or segment intraoral scans after an intraoral scanning session, and the segmentation/classification may be used to automatically determine presence and/or severity of dental conditions.

For each 3D model with labeled dental classes, a set of images (e.g., height maps) may be generated. Each image may be generated by projecting the 3D model (or a portion of the 3D model) onto a 2D surface or plane. Different images of a 3D model may be generated by projecting the 3D model onto different 2D surfaces or planes in some embodiments. For example, a first image of a 3D model may be generated by projecting the 3D model onto a 2D surface that is in a top down point of view, a second image may be generated by projecting the 3D model onto a 2D surface that is in a first side point of view (e.g., a buccal point of view), a third image may be generated by projecting the 3D model onto a 2D surface that is in a second side point of view (e.g., a lingual point of view), and so on. Each image may include a height map that includes a depth value associated with each pixel of the image. For each image, a probability map or mask may be generated based on the labeled dental classes in the 3D model and the 2D surface onto which the 3D model was projected. The probability map or mask may have a size that is equal to a pixel size of the generated image. Each point or pixel in the probability map or mask may include a probability value that indicates a probability that the point represents one or more dental classes. For example, there may be three dental classes, including a first dental class representing caries, a second dental class representing healthy teeth, and a third dental class representing gums. Points that have a first dental class may have a value of (1,0,0) (100% probability of first dental class and 0% probability of second and third dental classes), points that have a second dental class may have a value of (0,1,0), and points that have a third dental class may have a value of (0,0,1), for example.

A training dataset may be gathered, where each data item in the training dataset may include an image (e.g., an image comprising a height map) and an associated probability map. Additional data may also be included in the training data items. Accuracy of segmentation can be improved by means of additional classes, inputs and multiple views support. Multiple sources of information can be incorporated into model inputs and used jointly for prediction. Multiple dental classes can be predicted concurrently from a single model. Multiple problems can be solved simultaneously: teeth/gums segmentation, dental condition classification, etc. Accuracy is higher than traditional image and signal processing approaches.

Additional data may include a color image. For example, for each image (which may be a monochrome), there may also be a corresponding color image. Each data item may include depth information (e.g., a height map) as well as color information (e.g., from a color image). Two different types of color images may be available. One type of color image is a viewfinder image, and another type of color image is a scan texture. A scan texture may be a combination or blending of multiple different viewfinder images. Each intraoral scan may be associated with a corresponding viewfinder image generated at about the same time that the intraoral image was generated. If blended scans are used, then each scan texture may be based on a combination of viewfinder images that were associated with the raw scans used to produce a particular blended scan.

Another type of additional data may include an image generated under specific lighting conditions (e.g., an image generated under ultraviolet, near infrared or infrared lighting conditions). The additional data may be a 2D or 3D image, and may or may not include depth information (e.g., a height map).

The result of this training is a function that can predict dental classes directly from intraoral data (e.g., height maps of intraoral objects). In particular, the machine learning model(s) may be trained to generate a probability map, where each point in the probability map corresponds to a pixel of an input image and/or other input intraoral data and indicates one or more of a first probability that the pixel represents a first dental class, a second probability that the pixel represents a second dental class, a third probability that the pixel represents a third dental class, a fourth probability that the pixels represents a fourth dental class, a fifth probability that the pixel represents a fifth dental class, and so on.

From the dental diagnostics summary 103, a dentist may select any of the types of dental classes. For example, the dentist may select any one of tooth cracks 134, caries 120, gum recession 122, tooth wear 124, occlusion 126, crowding/spacing 128, plaque 130 and/or tooth stains 132. As discussed, multiple different types of dental conditions may be displayed, and for each type of dental condition a severity level for that dental condition may be shown. In the illustrated example, caries 120, tooth wear 124 and crowding/spacing 128 are shown to have issues found 155. Accordingly, as a result of performing a caries analysis, a tooth wear analysis and a crowding and/or spacing analysis, severity levels for tooth crowding/spacing 128, tooth wear 124 and caries 120 exceeded respective severity level thresholds. In the illustrated example, tooth stains 132 and occlusion 126 (e.g., poor occlusal contacts) are shown to have potential issues found 150, and tooth cracks 134, gum recession 122 and plaque 130 are shown to have no issues found 145.

A dentist, after a quick glance at the dental diagnostics summary 103, may determine that a patient has caries, clinically significant tooth wear, and crowding/spacing and/or other malocclusions. Accordingly, the dentist may select the caries 120 view option, the tooth wear 124 view option and/or the crowding/spacing 128 view option to quickly review the areas on the patient's dental arches at which caries, tooth wear and/or crowding (and/or spacing) were detected. The dentist may determine not to review gum recession, tooth cracks or plaque for the patient due to these dental conditions being classified as having no issues found. The dentist may or may not review the tooth stains and occlusion information due to these dental conditions having been classified as having potential issues found. Each of the illustrated dental conditions may be shown with an icon, button, link, or selectable option that a user can select via a graphical user interface of the dental diagnostics hub. Clicking on or otherwise selecting a particular dental condition may enable one or more tools associated with that specific dental condition.

The tools available to assess a selected dental condition may depend on the dental condition selected. For example, different assessment tools may be available for tooth stains 132 than for caries 120. In general, one of the available tools associated with a selected dental condition includes a simulation of a prognosis of the dental condition. Via the simulation, a doctor may determine what the area of interest (or areas of interest) exhibiting the dental condition looked like in the past and what they are predicted to look like in the future.

In at least one embodiment, the dental diagnostics hub, and in particular the dental diagnostics summary 103, helps a doctor to quickly detect dental conditions and their respective severity levels, helps the doctor to make better judgments about treatment of dental conditions, and further helps the doctor in communicating with a patient that patient's dental conditions and possible treatments. This makes the process of identifying, diagnosing, and treating dental conditions easier and more efficient. The doctor may select any of the dental conditions to determine prognosis of that condition as it exists in the present and how it will likely progress into the future. Additionally, the dental diagnostics hub may provide treatment simulations of how the dental conditions will be affected or eliminated by one or more treatments.

In at least one embodiment, a doctor may customize the dental conditions and/or areas of interest by adding emphasis or notes to specific dental conditions and/or areas of interest. For example, a patient may complain of a particular tooth aching. The doctor may highlight that particular tooth on the 3D model of the dental arches. Dental conditions that are found that are associated with the particular highlighted or selected tooth may then be shown in the dental diagnostics summary. In a further example, a doctor may select a particular tooth (e.g., lower left molar), and the dental diagnostics summary may be updated by modifying the severity results to be specific for that selected tooth. For example, if for the selected tooth an issue was found for caries and a possible issue was found for tooth stains, then the dental diagnostics summary 103 would be updated to show no issues found for tooth wear 124, occlusion 126, crowding/spacing 128, plaque 130, tooth cracks 134, and gum recession 122, to show a potential issue found for tooth stains 132 and to show an issue found for caries 120. This may help a doctor to quickly identify possible root causes for the pain that the patient complained of for the specific tooth that was selected. The doctor may then select a different tooth to get a summary of dental issues for that other tooth. Additionally, the doctor may select a dental arch, a quadrant of a dental arch, or a set of teeth, and the dental diagnostics summary 103 may be updated to show the dental conditions associated with the selected set of teeth, quadrant of a dental arch, and/or dental arch.

FIG. 1D illustrates a user interface for navigating diagnostics results provided to a mobile device 158 by a dental diagnostics hub, in accordance with embodiments of the present disclosure. The dental diagnostics summary 103 generated by a dental diagnostics hub may be sent to a device of a patient, which may be a mobile device 158 or a traditionally stationary device. Examples of mobile devices include mobile phones, tablet computers, laptops, and so on. Examples of traditionally stationary devices include desktop computers, server computers, smart televisions, set top boxes, and so on. In at least one embodiment, a link to the dental diagnostics summary 103 may be sent to the device of the patient, and the patient may activate the link (e.g., click on the link) to access the dental diagnostics summary 103. In at least one embodiment, the underlying information that is summarized in the dental diagnostics summary 103 may also be accessible by the patient by selecting on one or more of the dental conditions in the dental diagnostics summary 103. This may show the patient which teeth exhibit specific dental conditions, for example. The patient's version of the dental diagnostics summary 103 may further include or be associated with a schedule appointment option or function 160. The patient may click on or otherwise select the schedule appointment option or function 160 to schedule an appointment. This may cause a mobile phone to call a dentist office for example, or may cause the patient's device to navigate to a calendar view showing available appointment times. The patient may click on or otherwise select an available appointment time to schedule an appointment with their dentist.

FIG. 1E illustrates a user interface of a dental diagnostics hub showing a dental diagnostics summary 161 after diagnostics have been run on a patient's dental arches, in accordance with embodiments of the present disclosure. The dental diagnostics summary 161 presents dental information about a patient organized in a different manner than is shown in dental diagnostics summary 103. For dental diagnostics summary 103, summary information for many different types of dental conditions are shown together, without grouping the summary information based on dental categories. Dental diagnostics summary 161, on the other hand, groups dental conditions based on dental categories, and indicates specific types of dental conditions or problems within each of the dental categories. The dental condition information may also be arranged and presented in many other ways than the few examples shown herein.

In one embodiment, dental diagnostics summary 161 includes multiple high level dental categories or groups, including a restorative/prosthodontic category 162, a TMJ category 188, an orthodontic category 174, a periodontal category 164 and an endodontic category 182. All restorative and/or prosthodontic dental conditions may be displayed under restorative/prosthodontic category 162, all dental conditions associated with or caused by problems with TMJ may be displayed under TMJ category 188, all orthodontic dental conditions may be displayed under orthodontic category 174, all periodontal dental conditions may be displayed under periodontal category 164, and all endodontic dental conditions may be displayed under the endodontic category 182. Each of the high level dental categories may be coded (e.g., color coded) or otherwise include indicators to show whether or not dental conditions falling under those high level categories have been detected and/or severity levels of such dental conditions.

In at least one embodiment, one or more of the high level dental categories (e.g., restorative/prosthodontic category 162, TMJ category 188, orthodontic category 174, periodontal category 164, and endodontic category 182) include summary information for subcategories and/or particular dental conditions falling within the respective high level dental categories. In one embodiment, TMJ category 188 includes a cracks dental condition 190, an occlusion dental condition 192 and a tooth wear dental condition 194, which may correspond to tooth cracks 134, occlusion 126 and tooth wear 124, respectively, of FIG. 1C. For each of the cracks dental condition 190, occlusion dental condition 192 and tooth wear dental condition 194, the dental diagnostics summary 161 may indicate whether teeth of the patient are affected by the respective dental condition, which teeth are affected by the respective dental condition and/or a severity of the respective dental condition. Each of dental conditions within the TMJ category 188 may be coded (e.g., color coded) or otherwise include indicators to show whether or not the respective dental conditions fa have been detected and/or severity levels of such dental conditions.

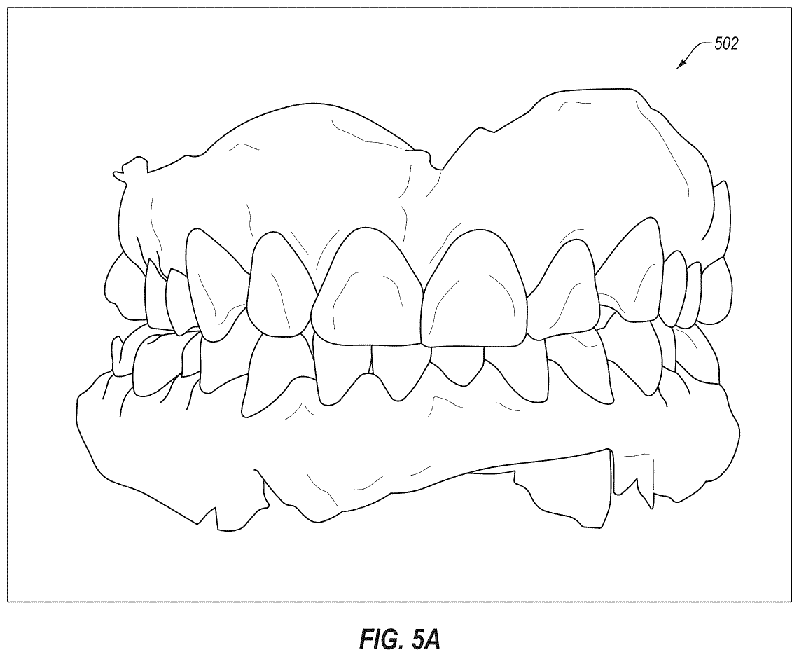

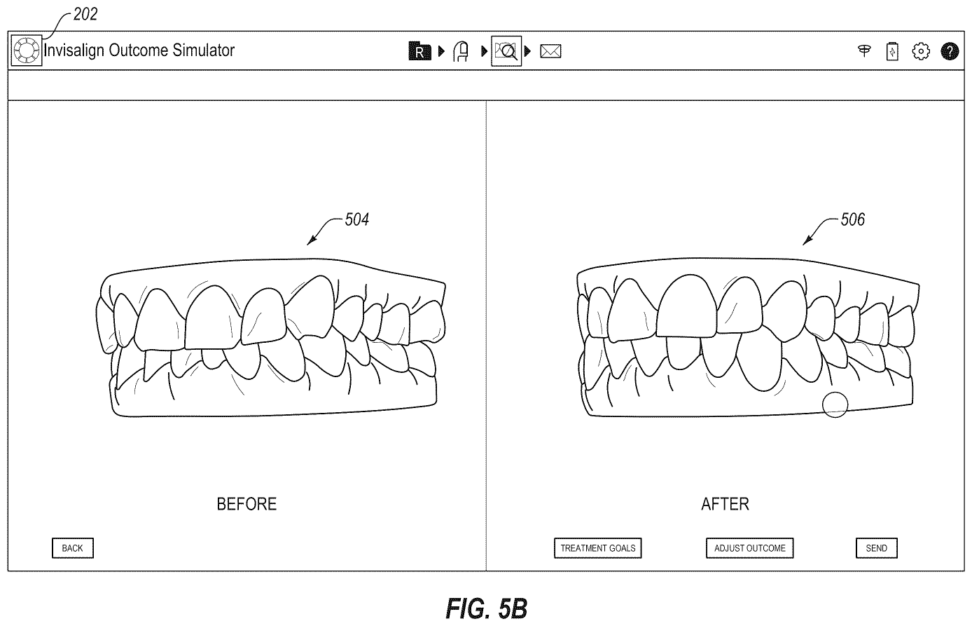

In one embodiment, orthodontic category 174 includes a crowding dental condition 176, a spacing dental condition 178 and a jaw discrepancies dental condition 180. Crowding dental condition 176 may correspond to crowding/spacing 128 of FIG. 1C. Spacing dental condition 178 may provide information on gaps or spaces between teeth of a patient. Jaw discrepancies dental condition 180 may include information on problems with a patient's jaw, such as how the jaw closes, overbite, underbite, overjet, and so on. For each of the crowding dental condition 176, spacing dental condition 178 and jaw discrepancies dental condition 180, the dental diagnostics summary 161 may indicate whether teeth of the patient are affected by the respective dental condition, which teeth are affected by the respective dental condition and/or a severity of the respective dental condition. Each of dental conditions within the TMJ category 188 may be coded (e.g., color coded) or otherwise include indicators to show whether or not the respective dental conditions have been detected and/or severity levels of such dental conditions. Selection of any of the spacing dental condition category 178, jaw discrepancies dental condition category 180 or crowding dental condition category 176 may launch a dental analysis tool illustrating the respective dental condition that was selected on the patient's dentition. From any of those dental analysis tools, an orthodontics tool may be launched, as discussed with reference to FIG. 5 below.

In one embodiment, periodontal category 164 includes an inflammation dental condition category 166, a bone loss dental condition category 170 and a gum recession dental condition category 167. Gum recession dental condition category 167 may correspond to gum recession 122 of FIG. 1C. Inflammation dental condition category 166 may include information on gum swelling or inflammation for one or more teeth and/or a degree of swelling. Bone loss dental condition category 170 may include information on bone density loss for one or more regions of a patient's jaw. For each of the gum recession dental condition 167, inflammation dental condition 166 and bone loss dental condition 170, the dental diagnostics summary 161 may indicate whether teeth of the patient are affected by the respective dental condition, which teeth are affected by the respective dental condition (e.g., tooth nos. 168, 169, 172) and/or a severity of the respective dental condition. Each of the dental conditions within the periodontal category 164 may be coded (e.g., color coded) or otherwise include indicators to show whether or not the respective dental conditions have been detected and/or severity levels of such dental conditions. Selection of any of the gum recession dental condition category 167, inflammation dental condition category 166 or bone loss dental condition category 170 may launch a dental analysis tool illustrating the respective dental condition that was selected on the patient's dentition.

In one embodiment, endodontic category 182 includes one or more types of endodontic problems. Endodontic problems may include problems relating to tooth roots and the soft tissues inside a tooth, such as dental pulp in a tooth. Endodontic category 182 may include endodontic conditions for one or more problem types 184, such as a first problem type for problems with dental pulp and a second problem type for problems with tooth roots. For each problem type 184, one or more affected tooth numbers 186 may be indicated. Each of dental conditions within the endodontic category 182 may be coded (e.g., color coded) or otherwise include indicators to show whether or not the respective dental conditions have been detected and/or severity levels of such dental conditions. Selection of any of the problem types 184 may launch a dental analysis tool illustrating the respective dental condition that was selected on the patient's dentition.

In one embodiment, restorative/prosthodontic category 162 includes one or more types of restorative and/or prosthodontic conditions. The term prosthodontic procedure refers, inter alia, to any procedure involving the oral cavity and directed to the design, manufacture or installation of a dental prosthesis at a dental site within the oral cavity, or a real or virtual model thereof, or directed to the design and preparation of the dental site to receive such a prosthesis. A prosthesis may include any restoration such as crowns, veneers, inlays, onlays, and bridges, for example, and any other artificial partial or complete denture. Prosthodontic dental conditions or issues may include a failing, failed or broken/cracked prosthesis, a worn prosthesis, a loose prosthesis, an ill-fitting prosthesis, and so on. Prosthodontic dental conditions may also include conditions that can be corrected by a prosthesis, such as a missing tooth, an edentulous dental arch, and so on. Restorative/prosthodontic category 162 may include conditions with existing prosthodontics, which may constitute a first problem type 163, and conditions that can be resolved using prosthodontics, which may constitute a second problem type 163. For each problem type 163, one or more affected tooth numbers 165 may be indicated. Each of the dental conditions within the restorative/prosthodontic category 162 may be coded (e.g., color coded) or otherwise include indicators to show whether or not the respective dental conditions have been detected and/or severity levels of such dental conditions. Selection of any of the problem types 163 may launch a dental analysis tool illustrating the respective dental condition that was selected on the patient's dentition.

In at least one embodiment, the dental diagnostics summary 161 includes the scan segment selector 102 including upper dental arch segment selection 105, lower dental arch segment selection 110 and/or bite segment selection 115. In at least one embodiment, the dental diagnostics summary 103 further includes views of the selected dental segments or modes (e.g., of the 3D model of the upper dental arch 140 and the 3D model of the lower dental arch 141 of the patient).

The dental diagnostics summary 161 provides a single view showing multiple different types of possible dental conditions at both a high level and at a lower level, and assessments as to the presence and/or severity of each of the types of dental conditions. In one embodiment, the various dental conditions are assigned one of three severity levels, including “no issues found”, “potential issues found” and “issues found”. Each of the dental conditions and/or dental categories (e.g., high level categories that may include multiple underlying conditions) may be coded or labeled with the severity ranking determined for that type of dental condition. In one embodiment, the dental conditions and/or categories are color coded to graphically show severity levels. For example, those dental conditions and/or categories for which issues were found may be coded red, those dental conditions and/or categories for which potential issues were found may be coded yellow, and those dental conditions and/or categories for which no issues were found may be coded green. Many other coding schemes are also possible. In one embodiment, each of the dental conditions and/or categories is assigned a numeric severity level. For example, on a scale of 1 to 100, each dental condition and/or category may be assigned a severity level between 1 and 100 to indicate the severity level of that dental condition. Those dental conditions and/or categories with a severity level that is below a first threshold severity level may be identified as dental conditions for which no issues were found. Those dental conditions and/or categories for which the severity level is above the first threshold severity level but below a second threshold severity level may be identified as dental conditions/categories for which potential issues were found. Those dental conditions and/or categories for which the severity level is above the second severity level threshold may be identified as dental conditions/categories for which issues were found.

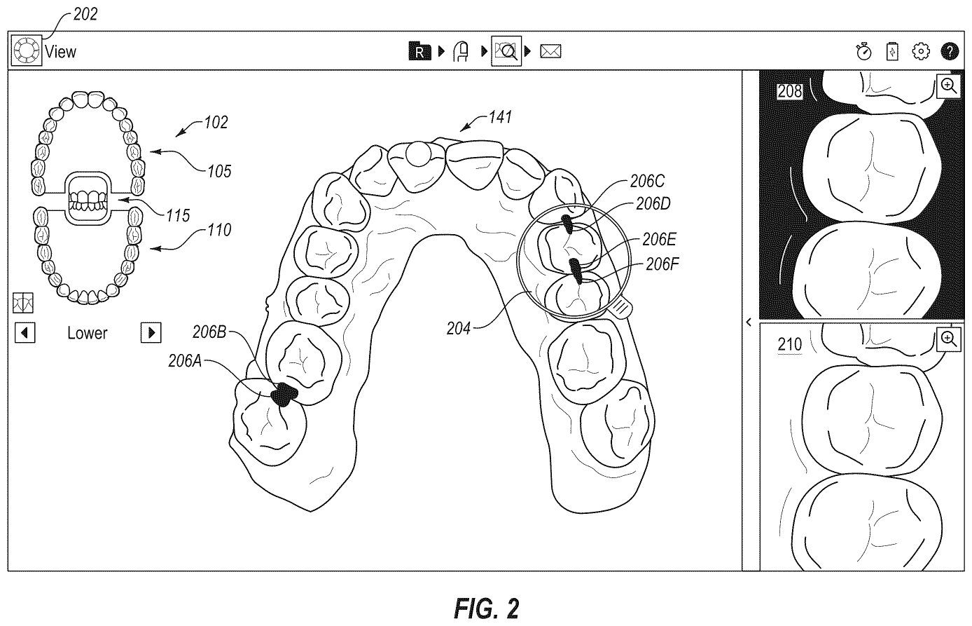

FIG. 2 illustrates a user interface for a caries analysis of a dental diagnostics hub, in accordance with embodiments of the present disclosure. The user interface for caries analysis may be provided responsive to a doctor selecting caries from the dental diagnostics summary 103. In at least one embodiment, the caries analysis user interface includes the scan segment selector 102 including upper dental arch segment selection 105, lower dental arch segment selection 110 and/or bite segment selection 115. In at least one embodiment each tooth that has an area of interest associated with the selected dental condition is highlighted or otherwise emphasized or marked in the scan segment selector 102. Accordingly, a quick glance at the scan segment selector 102 may show a doctor where to look further to review the AOIs with the selected dental condition. In at least one embodiment individual teeth may be selected in the scan segment selector 102 to view just those selected teeth. For example, a doctor may select one or a few teeth having AOIs to show a 3D model with just those teeth.

In at least one embodiment, the user interface for caries analysis further includes views of the selected dental segments or modes. In the illustrated example, a lower dental arch is selected, and the 3D model of the lower dental arch 141 of the patient is shown. An overlay of areas of interest (AOIs) that reflect detected caries is shown on the 3D model of the lower dental arch. For example, areas of interest 206A-F representing detected caries are shown on the 3D model of the lower dental arch 141. The upper dental arch segment may be selected to view AOIs representing caries in the upper dental arch. Additionally, both the upper and lower dental arch may be selected to show caries on both the upper and lower dental arches.

A doctor may change a view of the displayed 3D model or 3D models (e.g., of the 3D model of the lower dental arch 141) via the user interface so as to better view identified AOIs. Such changes to the view may include changing a zoom setting (e.g., by zooming in or out), rotating the 3D model(s), panning left, right, up, down, etc., and so on. A doctor may additionally use a focus tool to move a focus window 204 anywhere on the 3D model to focus in on a region of the 3D model of the dental arch(es). Additional information from one or more additional imaging modalities may be shown for a region that is within the focus window 204. For example, NIRI data for the region may be shown in a NIRI window 208, and color data for the region may be shown in a color window 210. For both the NIRI window 208 and the color window 210 the doctor may zoom in or out and/or change a view of the region.

The doctor may select a time-based simulation function to launch a time-based simulation for the selected dental condition (e.g., for caries). The time-based simulation may use information about AOIs as they existed at different points in time from the patient's record history (e.g., intraoral scans, NIRI images, color images, x-rays, etc. from different points in time) to project progression of the dental condition into the future and/or into the past. The time-based simulation may generate a video showing the start of the dental condition and progression of the dental condition over time to the present status of the dental condition and into the future. The time-based simulation may further include one or more treatment options, and may show what the areas of interest into the future after one or more selected treatments are performed.

The user interface for the caries analysis may indicate, for each of the detected caries 206A-F, a severity level of the caries. The severity level may be based on a size of the caries, on a location of the caries and/or on a distance between the caries and a patient's dentin and/or pulp.

In at least one embodiment a secure share mode may be provided in which doctors can collaborate securely with other care providers and/or can communicate securely with patients (or parents of patients) via a remote connection.

A doctor may select a learn mode option (not shown) to bring up educational information on the difference between healthy teeth and teeth having caries, and the difference between different severity levels of caries. The patient's current dentition with currently detected caries may be shown, and further tooth decay may be projected. The educational information may show what happens when the tooth decay reaches the patient's dentin and/or pulp, indicating an amount of pain that the patient can expect at various stages of tooth decay. The educational information may be shown to a patient to show that patient the stages of tooth decay for their teeth and what will happen if they don't treat the tooth decay.

Once the doctor is done reviewing the caries information for the patient, the doctor may select a dental diagnostics summary view icon or navigation option 202 to navigate back to the dental diagnostic summary 103.

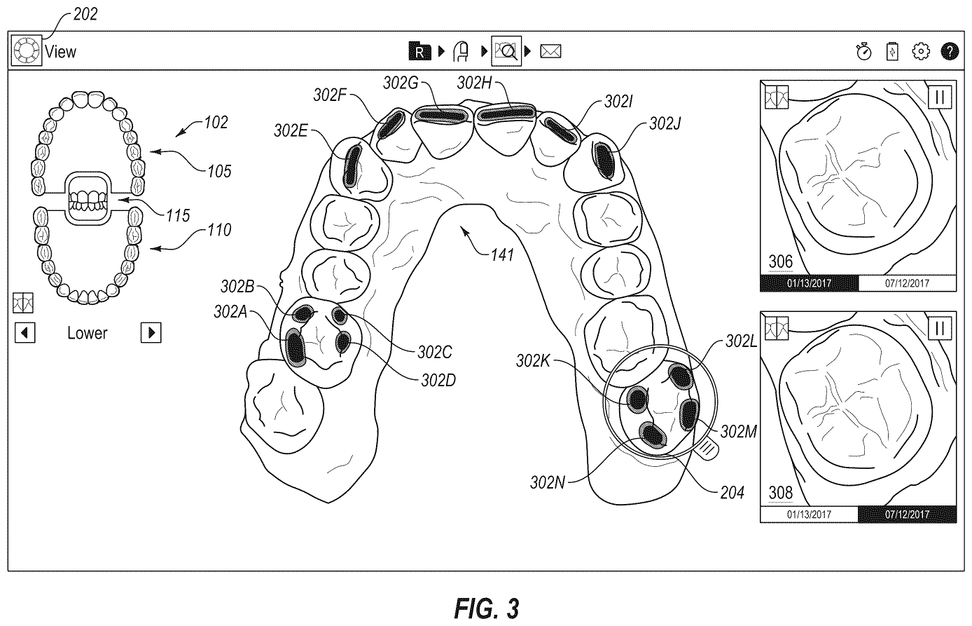

FIG. 3 illustrates a user interface for a tooth wear analysis of a dental diagnostics hub, in accordance with embodiments of the present disclosure. The user interface for tooth wear analysis may be provided responsive to a doctor selecting tooth wear from the dental diagnostics summary 103. In at least one embodiment, the tooth wear analysis user interface includes the scan segment selector 102 including upper dental arch segment selection 105, lower dental arch segment selection 110 and/or bite segment selection 115. In at least one embodiment each tooth that has an area of interest associated with the selected dental condition is highlighted or otherwise emphasized or marked in the scan segment selector 102. Accordingly, a quick glance at the scan segment selector 102 may show a doctor where to look further to review the AOIs with the selected dental condition. In at least one embodiment individual teeth may be selected in the scan segment selector 102 to view just those selected teeth. For example, a doctor may select one or a few teeth having AOIs to show a 3D model with just those teeth.

In at least one embodiment, the user interface for tooth wear analysis further includes views of the selected dental segments or modes. In the illustrated example, a lower dental arch is selected, and the 3D model of the lower dental arch 141 of the patient is shown. An overlay of areas of interest (AOIs) that reflect detected tooth wear is shown on the 3D model of the lower dental arch. For example, areas of interest 302A-N representing detected regions of tooth wear are shown on the 3D model of the lower dental arch 141. The upper dental arch segment may be selected to view AOIs representing tooth wear in the upper dental arch. Additionally, both the upper and lower dental arch may be selected to show tooth wear on both the upper and lower dental arches.

A doctor may change a view of the displayed 3D model or 3D models (e.g., of the 3D model of the lower dental arch 141) via the user interface so as to better view identified AOIs. Such changes to the view may include changing a zoom setting (e.g., by zooming in or out), rotating the 3D model(s), panning left, right, up, down, etc., and so on. A doctor may additionally use a focus tool to move a focus window 204 anywhere on the 3D model to focus in on a region of the 3D model of the dental arch(es). Additional information from one or more additional imaging modalities may be shown for a region that is within the focus window 404. For example, a 3D surface of the region within the focus window 204 may be shown for a first time period in a first point-in-time snapshot window 306 and for a second time period in a second point-in-time snapshot window 308. For each of the point-in-time snapshot windows 306, 308, a doctor may select a specific point-in-time snapshot to view. The point-in-time snapshots may show scanned surfaces for points-in-time at which scans were performed and/or may show interpolated or extrapolated surfaces for points-in-time at which no scans were performed. For each of the point-in-time snapshot windows, a doctor may press a play or pause icon or button to show a progression of the 3D surfaces from the current selected point-of-time into the future and/or into the past.

The doctor may select the time-based simulation function to launch a time-based simulation for the selected dental condition (e.g., for tooth wear). The time-based simulation may use information about AOIs as they existed at different points in time from the patient's record history (e.g., intraoral scans, NIRI images, color images, x-rays, etc. from different points in time) to project progression of the dental condition into the future and/or into the past. The time-based simulation may generate a video showing the start of the dental condition and progression of the dental condition over time to the present status of the dental condition and into the future. The time-based simulation may further include one or more treatment options, and may show what the areas of interest into the future after one or more selected treatments are performed.

Once the doctor is done reviewing the tooth wear information for the patient, the doctor may select the dental diagnostics summary view icon or navigation option 202 to navigate back to the dental diagnostic summary 103.

No user interfaces are shown for gum swelling, plaque, tooth cracks or gum recession. However, similar views for gum swelling, plaque, tooth cracks and/or gum recession may be shown to a dentist as are shown with regards to caries and tooth wear. Additionally, similar dental condition analysis tools may be provided for gum swelling, plaque, tooth cracks and/or gum recession as are provided for caries and/or tooth wear. A gum swelling analysis tool, for example, may project an amount of gum swelling into the future, and show inflammation of the gums, gum bleeding, and so on. Similarly, a gum recession analysis tool may project an amount of gum recession into the future, showing exposed portions of tooth roots, and so on.

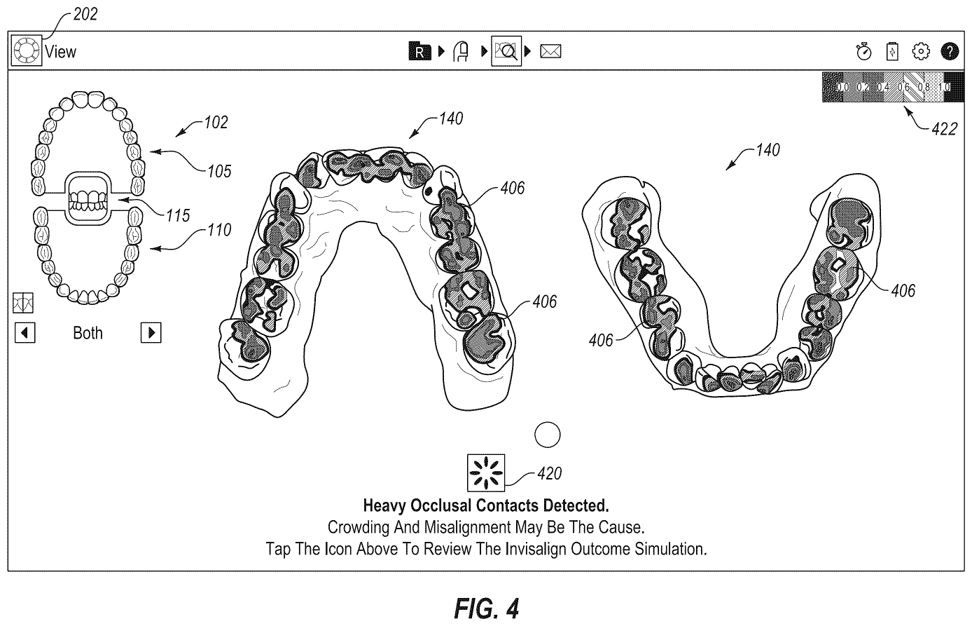

FIG. 4 illustrates a user interface for an occlusal contact analysis of a dental diagnostics hub, in accordance with embodiments of the present disclosure. The user interface for occlusal contact analysis may be provided responsive to a doctor selecting occlusal contacts from the dental diagnostics summary 103. In at least one embodiment, the occlusal contact analysis user interface includes the scan segment selector 102 including upper dental arch segment selection 105, lower dental arch segment selection 110 and/or bite segment selection 115. In at least one embodiment each tooth that has an area of interest associated with the selected dental condition is highlighted or otherwise emphasized or marked in the scan segment selector 102. Areas of interest for occlusal contacts may be tooth regions for which heavy occlusal contacts are identified, for example. Accordingly, a quick glance at the scan segment selector 102 may show a doctor where to look further to review the AOIs with the selected dental condition. In at least one embodiment individual teeth may be selected in the scan segment selector 102 to view just those selected teeth. For example, a doctor may select one or a few teeth having AOIs to show a 3D model with just those teeth.

In at least one embodiment, the user interface for occlusal contact analysis further includes views of the selected dental segments or modes. In the illustrated example, both an upper dental arch segment and a lower dental arch are selected, and the 3D models of the lower dental arch 141 and of the upper dental arch 140 of the patient are shown. An overlay of areas of interest (AOIs) 406 that reflect detected occlusal contact is shown on the 3D models of the lower dental arch 141 and of the upper dental arch 140. The occlusal contact overlay may be coded in some way (e.g., color coded) to show the severity or amount of occlusal contact for one or more regions of the teeth on the dental arches. The coding may indicate a distance from 0 to 1, where 0 represents full contact with the opposing dental arch (e.g., in which the AOI is in contact for multiple different relative positions of the upper and lower jaws) and 1 represents full separation from the opposing dental arch (e.g., in which the AOI is not in contact for any of the multiple different relative positions of the upper and lower jaws). A legend 422 for the coding may be shown in the user interface, showing different colors or other graphical indicators for each of the occlusal contact ratings.

The user interface for the occlusal contact analysis may include information on possible underlying causes of problematic occlusal contacts, such as crowding and misalignment of teeth. From the user interface for the occlusal contact analysis, the doctor may select an orthodontic treatment 420 icon or button to launch an orthodontic treatment simulator and/or orthodontic treatment plan generator.

A doctor may change a view of the displayed 3D model or 3D models (e.g., of the 3D model of the lower dental arch 141 and upper dental arch 140) via the user interface so as to better view identified AOIs. Such changes to the view may include changing a zoom setting (e.g., by zooming in or out), rotating the 3D model(s), panning left, right, up, down, etc., and so on.

The doctor may select the time-based simulation function to launch a time-based simulation for the selected dental condition (e.g., for occlusal contacts). The time-based simulation may use information about AOIs as they existed at different points in time from the patient's record history (e.g., intraoral scans, NIRI images, color images, x-rays, etc. from different points in time) to project progression of the dental condition into the future and/or into the past. The time-based simulation may generate a video showing the start of the dental condition and progression of the dental condition over time to the present status of the dental condition and into the future. The time-based simulation may further include one or more treatment options, and may show what the areas of interest into the future after one or more selected treatments are performed.

Once the doctor is done reviewing the occlusal contact information for the patient, the doctor may select the dental diagnostics summary view icon or navigation option 202 to navigate back to the dental diagnostic summary 103.

FIG. 5A illustrates a user interface for malocclusion analysis of a dental diagnostics hub, in accordance with embodiments of the present disclosure. The user interface for malocclusion analysis may be provided responsive to a doctor selecting crowding from the dental diagnostics summary 103. In at least one embodiment, the malocclusion analysis user interface includes the scan segment selector 102 including upper dental arch segment selection 105, lower dental arch segment selection 110 and/or bite segment selection 115. In at least one embodiment each tooth that has an area of interest associated with the selected dental condition is highlighted or otherwise emphasized or marked in the scan segment selector 102. Areas of interest for occlusal contacts may be tooth regions for which heavy occlusal contacts are identified, for example. Accordingly, a quick glance at the scan segment selector 102 may show a doctor where to look further to review the AOIs with the selected dental condition. In at least one embodiment individual teeth may be selected in the scan segment selector 102 to view just those selected teeth. For example, a doctor may select one or a few teeth having AOIs to show a 3D model with just those teeth.

AOIs showing tooth crowding 501 may be shown on the 3D models of the upper dental arch 140 and/or lower dental arch 141.

From the user interface for the malocclusion analysis, the doctor may select an orthodontic treatment 420 icon or button to launch an orthodontic treatment simulator and/or orthodontic treatment plan generator.

A doctor may change a view of the displayed 3D model or 3D models (e.g., of the 3D model of the lower dental arch 141 and upper dental arch 140) via the user interface so as to better view identified AOIs. Such changes to the view may include changing a zoom setting (e.g., by zooming in or out), rotating the 3D model(s), panning left, right, up, down, etc., and so on.

The doctor may select the time-based simulation function to launch a time-based simulation for the selected dental condition (e.g., for malocclusion).

Once the doctor is done reviewing the malocclusion information for the patient, the doctor may select the dental diagnostics summary view icon or navigation option 202 to navigate back to the dental diagnostic summary 103.

FIG. 5B illustrates an orthodontic treatment simulator user interface showing pre-treatment and post-treatment models of a dental arch, in accordance with embodiments of the present disclosure. The orthodontic treatment simulator may be launched responsive to a doctor selecting an orthodontic treatment 420 icon or button, such as from the user interface for malocclusion analysis and/or the user interface for occlusal contact analysis.

The orthodontic treatment simulator may determine a movement path to move one or more teeth from an initial arrangement of teeth 504 as determined from 3D models of a patient's current dental arches to a target arrangement of teeth 506 for the patient's dental arches. The target arrangement of the teeth (e.g., a desired and intended end result of orthodontic treatment) can be received from a clinician in the form of a prescription, can be calculated from basic orthodontic principles, and/or can be extrapolated computationally from a clinical prescription. In at least one embodiment, a target arrangement for the patient's teeth is automatically determined based on aesthetic principles and/or ideal occlusion principles.

A doctor may adjust treatment goals via option 510, which may cause the target arrangement of the teeth 506 to change. Additionally, the doctor may directly or manually adjust the simulated treatment outcome via option 515.

With a specification of the desired final positions of the teeth and a digital representation of the teeth themselves, the final position and surface geometry of each tooth can be specified to form a complete model of the tooth arrangement at the desired end of treatment.

Having both an initial position and a target position for each tooth, a movement path can be defined for the motion of each tooth. In at least one embodiment the movement paths are configured to move the teeth in the quickest fashion with the least amount of round-tripping to bring the teeth from their initial positions to their desired target positions. The tooth paths can optionally be segmented, and the segments can be calculated so that each tooth's motion within a segment stays within threshold limits of linear and rotational translation. In this way, the end points of each path segment can constitute a clinically viable repositioning, and the aggregate of segment end points can constitute a clinically viable sequence of tooth positions, so that moving from one point to the next in the sequence does not result in a collision of teeth.

A force system to produce movement of the one or more teeth along the movement path may be determined. A force system can include one or more forces and/or one or more torques. Different force systems can result in different types of tooth movement, such as tipping, translation, rotation, extrusion, intrusion, root movement, etc. Biomechanical principles, modeling techniques, force calculation/measurement techniques, and the like, including knowledge and approaches commonly used in orthodontia, may be used to determine the appropriate force system to be applied to the tooth to accomplish the tooth movement. In determining the force system to be applied, sources may be considered including literature, force systems determined by experimentation or virtual modeling, computer-based modeling, clinical experience, minimization of unwanted forces, etc.

The initial arrangement of teeth 504 and/or target arrangement of teeth 506 for the patient's dental arches may be sent to a patient for their review via option 520. A doctor may navigate back to a previous interface, such as to the malocclusion analysis user interface or to the user interface for occlusal contact analysis, via the back option 525. Additionally, once the doctor is done reviewing the simulated orthodontic treatment results, the doctor may select the dental diagnostics summary view icon or navigation option 202 to navigate back to the dental diagnostic summary 103.

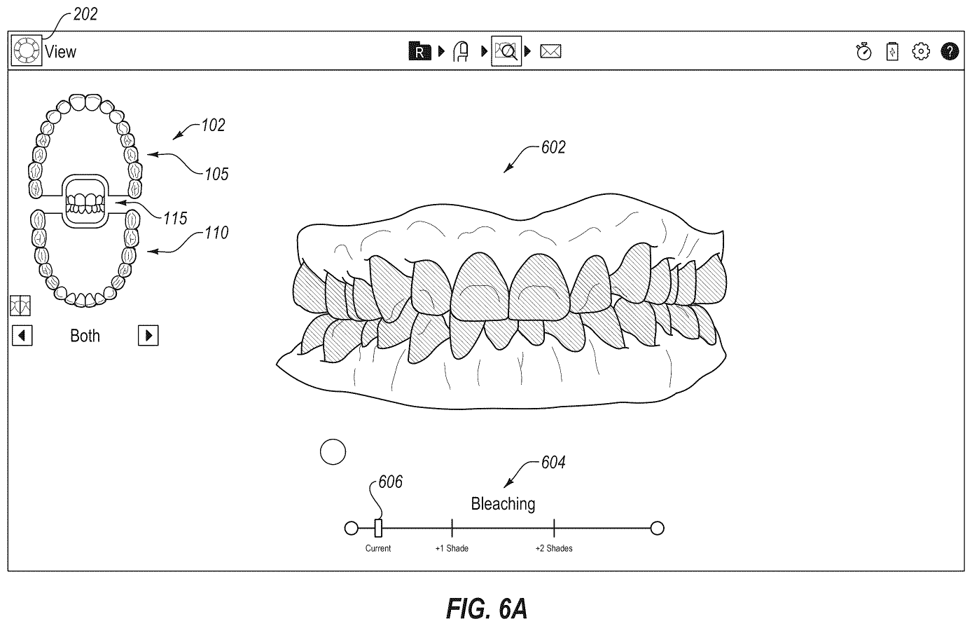

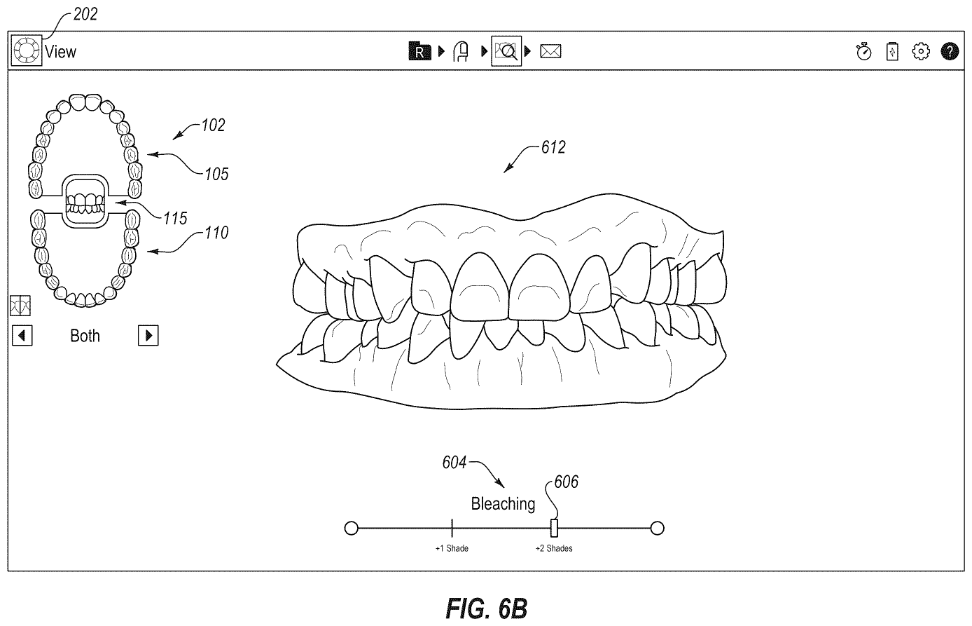

FIGS. 6A-B illustrate a user interface for a tooth stain analysis of a dental diagnostics hub, in accordance with embodiments of the present disclosure. The user interface for tooth stains analysis may be provided responsive to a doctor selecting tooth stains from the dental diagnostics summary 103. In at least one embodiment, the tooth stains analysis user interface includes the scan segment selector 102 including upper dental arch segment selection 105, lower dental arch segment selection 110 and/or bite segment selection 115.

In at least one embodiment, the user interface for tooth stains analysis further includes views of the selected dental segments or modes. In the illustrated example, both an upper dental arch segment and a lower dental arch are selected, and the 3D models of the lower dental arch 141 and of the upper dental arch 140 of the patient are shown. The 3D models of the upper dental arch 140 and/or lower dental arch 141 are shown with accurate color information showing the current color of the patient's teeth (which may include staining).

The user interface for the tooth staining analysis includes a tooth bleaching selector 604, which may be a slide bar in embodiments. In FIG. 6A, the tooth bleaching selector 604 indicates a current shade indicator 606 for the teeth. The current shade indicator 606 shows the current tooth shade on a tooth shade spectrum. As shown, the current shade indicator 606 shows that the patient's teeth are significantly stained. The doctor may adjust the current shade indicator 606 to one or more different tooth shades. In response to the current shade indicator 606 being adjusted along the tooth bleaching slide bar, processing logic determines updated color information for the teeth, and displays the teeth with the updated color information. FIG. 6B illustrates the user interface for the tooth stain analysis showing updated shading of the patient's teeth after the doctor has moved the current shade indicator 606 to +2 shades.

A doctor may change a view of the displayed 3D model or 3D models (e.g., of the 3D model of the lower dental arch 141 and upper dental arch 140) via the user interface so as to better view identified AOIs. Such changes to the view may include changing a zoom setting (e.g., by zooming in or out), rotating the 3D model(s), panning left, right, up, down, etc., and so on.

The doctor may select the time-based simulation function to launch a time-based simulation for the selected dental condition (e.g., for tooth staining). The time-based simulation may use information about AOIs as they existed at different points in time from the patient's record history (e.g., intraoral scans, NIRI images, color images, x-rays, etc. from different points in time) to project progression of the dental condition into the future and/or into the past. The time-based simulation may generate a video showing the start of the dental condition and progression of the dental condition over time to the present status of the dental condition and into the future. The time-based simulation may further include one or more treatment options, and may show what the areas of interest into the future after one or more selected treatments are performed.

Once the doctor is done reviewing the occlusal contact information for the patient, the doctor may select the dental diagnostics summary view icon or navigation option 202 to navigate back to the dental diagnostic summary 103.

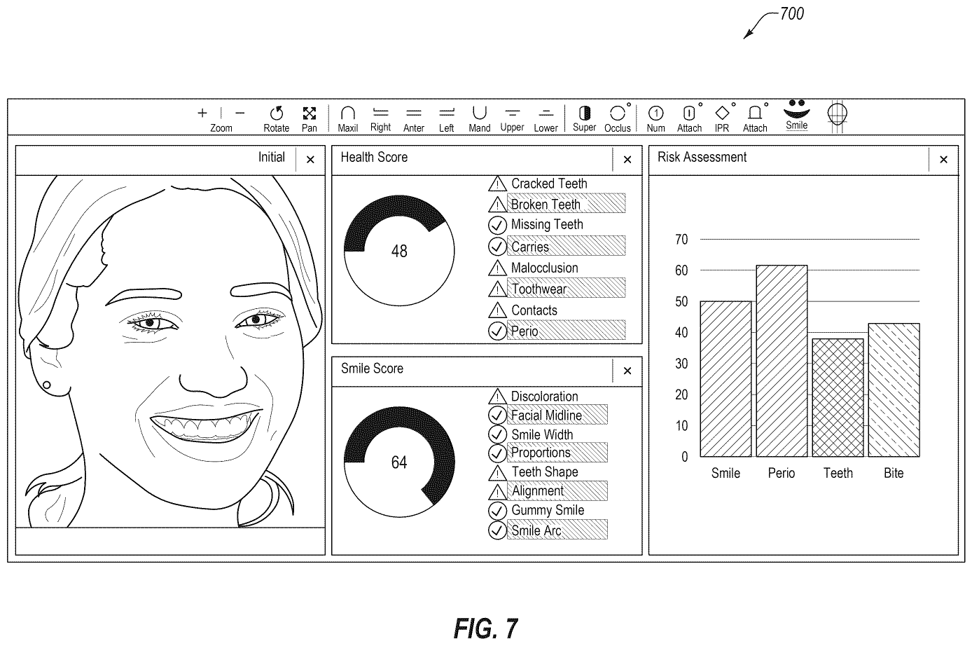

FIG. 7 illustrates a patient dental score card 700 of a dental diagnostics hub, in accordance with embodiments of the present disclosure. The patient dental score card 700 may include an image of the patient's smile 702, a dental health score 704, a dental smile score 706 and a dental risk assessment 708. The dental health score 704 may be based on a result of the multiple dental condition analyses performed by the dental diagnostics hub, and may be a global score reflective of patient dental health based on a combination of the various types of identified dental conditions and the severity levels of the identified dental conditions. For example, the dental health score 704 may be based on one or more of an assessment of cracked teeth, broken teeth, missing teeth, caries, malocclusion, tooth wear, occlusal contacts, periodontal issues, and so on. Selection of the dental health score 704 may cause the dental diagnostics hub to navigate to the dental diagnostic summary 103 in embodiments. The smile score 706 may be based on one or more of an assessment of tooth staining or discoloration, a facial midline, smile width, dental and/or facial proportions, tooth shape, tooth alignment, amount of gums that show in smile and/or an arc of the smile. The risk assessment may show a smile score (associated with tooth staining and so on), a periodontal score associated with gum recession and/or gum swelling), a teeth score (associated with tooth cracks, tooth breaks, missing teeth and/or caries) and a bite score (associated with occlusal contacts).

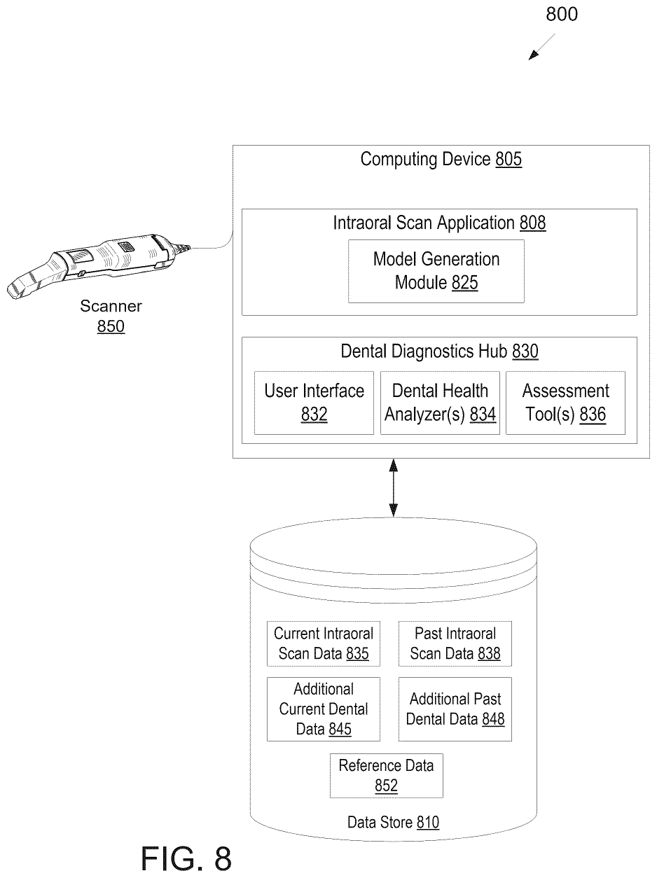

FIG. 8 illustrates one embodiment of a system 800 for performing intraoral scanning, generating a virtual three dimensional model of a dental site and/or performing dental diagnostics. In one embodiment, system 800 carries out one or more operations of below described with reference to FIGS. 1A-7 and 9A-12. System 800 includes a computing device 805 that may be coupled to a scanner 850 and/or a data store 810.

Computing device 805 may include a processing device, memory, secondary storage, one or more input devices (e.g., such as a keyboard, mouse, tablet, and so on), one or more output devices (e.g., a display, a printer, etc.), and/or other hardware components. Computing device 805 may be connected to a data store 810 either directly or via a network. The network may be a local area network (LAN), a public wide area network (WAN) (e.g., the Internet), a private WAN (e.g., an intranet), or a combination thereof. The computing device 805 may be integrated into the scanner 850 in some embodiments to improve performance and mobility.

Data store 810 may be an internal data store, or an external data store that is connected to computing device 805 directly or via a network. Examples of network data stores include a storage area network (SAN), a network attached storage (NAS), and a storage service provided by a cloud computing service provider. Data store 810 may include a file system, a database, or other data storage arrangement.

In at least one embodiment a scanner 850 for obtaining three-dimensional (3D) data of a dental site in a patient's oral cavity is operatively connected to the computing device 805. Scanner 850 may include a probe (e.g., a hand held probe) for optically capturing three dimensional structures (e.g., by confocal focusing of an array of light beams). One example of such a scanner 850 is the iTero® intraoral digital scanner manufactured by Align Technology, Inc. Other examples of intraoral scanners include the 8M™ True Definition Scanner and the Apollo DI intraoral scanner and CEREC AC intraoral scanner manufactured by Sirona®.

The scanner 850 may be used to perform an intraoral scan of a patient's oral cavity. An intraoral scan application 808 running on computing device 805 may communicate with the scanner 850 to effectuate the intraoral scanning. A result of the intraoral scanning may be a sequence of intraoral images or scans that have been generated. Each intraoral scan may include x, y and z position information for one or more points on a surface of a scanned object. In one embodiment, each intraoral scan includes a height map of a surface of a scanned object. An operator may start a scanning operation with the scanner 850 at a first position in the oral cavity, move the scanner 850 within the oral cavity to a second position while the scanning is being performed, and then stop recording of intraoral scans. In at least one embodiment recording may start automatically as the scanner identifies either teeth. The scanner 850 may transmit the intraoral scans to the computing device 805. Computing device 805 may store the current intraoral scan data 835 from a current scanning session in data store 810. Data store 810 may additionally include past intraoral scan data 838, additional current dental data 845 generated during a current patient visit (e.g., x-ray images, CBCT scan data, panoramic x-ray images, ultrasound data, color photos, and so on), additional past dental data generated during one or more prior patient visits (e.g., x-ray images, CBCT scan data, panoramic x-ray images, ultrasound data, color photos, and so on), and/or reference data 852. Alternatively, scanner 850 may be connected to another system that stores data in data store 810. In such an embodiment, scanner 850 may not be connected to computing device 805.

According to an example, a user (e.g., a practitioner) may subject a patient to intraoral scanning. In doing so, the user may apply scanner 850 to one or more patient intraoral locations. The scanning may be divided into one or more segments (e.g., upper dental arch, lower dental arch, and bite). Via such scanner application, the scanner 850 may provide the current intraoral scan data 835 to computing device 805. The current and/or past intraoral scan data 835, 838 may include 3D surface data (e.g., in the form of 3D images or images with height information), 2D or 3D color image data, NIRI image data, ultraviolet image data, and so on. Such scan data may be provided from the scanner to the computing device 805 in the form of one or more points (e.g., one or more pixels and/or groups of pixels). For instance, the scanner 850 may provide a 3D image as one or more point clouds.

In one embodiment, intraoral scan application 808 includes a model generation module 825. When a scan session is complete (e.g., all images for a dental site have been captured), model generation module 825 may generate a virtual 3D model of the scanned dental site. To generate the virtual model, model generation module 825 may register and “stitch” together the intraoral scans generated from the intraoral scanning session. In one embodiment, performing registration includes capturing 3D data of various points of a surface in multiple scans (views from a camera), and registering the scans by computing transformations between the images, as discussed herein above.

In one embodiment, computing device 805 includes a dental diagnostics hub 830, which may include a user interface 832, one or more dental health analyzers 834 and/or one or more dental condition assessment tools 836. The user interface 832 may be a graphical user interface and may include icons, buttons, graphics, menus, windows and so on for controlling and navigating the dental diagnostics hub 839.

Each of the dental health analyzers may be responsible for performing an analysis associated with a different type of dental condition. For example, dental health analyzers 834 may include separate dental health analyzers 834 for tooth cracks, gum recession, tooth wear, occlusal contacts, crowding of teeth and/or other malocclusions, plaque, tooth stains, and/or caries. In one embodiment, a single dental health analyzer 834 performs each of the different type of dental health analyses associated with each of the types of dental conditions discussed herein. In at least one embodiment there are multiple dental health analyzers, some of which perform dental health analysis for multiple different dental conditions. As discussed above, current intraoral scan data 835, past intraoral scan data 838, additional current dental data 845, additional past dental data 848 and/or reference data 852 may be used to perform one or more dental analysis. For example, the data regarding an at-hand patient may include X-rays, 2D intraoral images, 3D intraoral images, 2D models, and/or virtual 3D models corresponding to the patient visit during which the scanning occurs. The data regarding the at-hand patient may additionally include past X-rays, 2D intraoral images, 3D intraoral images, 2D models, and/or virtual 3D models of the patient (e.g., corresponding to past visits of the patient and/or to dental records of the patient).

Reference data 852 may include pooled patient data, which may include X-rays, 2D intraoral images, 3D intraoral images, 2D models, and/or virtual 3D models regarding a multitude of patients. Such a multitude of patients may or may not include the at-hand patient. The pooled patient data may be anonymized and/or employed in compliance with regional medical record privacy regulations (e.g., the Health Insurance Portability and Accountability Act (HIPAA)). The pooled patient data may include data corresponding to scanning of the sort discussed herein and/or other data. Reference data may additionally or alternatively include pedagogical patient data, which may include X-rays, 2D intraoral images, 3D intraoral images, 2D models, virtual 3D models, and/or medical illustrations (e.g., medical illustration drawings and/or other images) employed in educational contexts.

One or more dental health analyzers 834 may perform one or more types of dental condition analyses using intraoral data (e.g., current intraoral scan data 835, past intraoral scan data 838, additional current dental data 845, additional past dental data 848 and/or reference data 852), as discussed herein above. As a result, dental diagnostics hub 830 may determine multiple different dental conditions and severity levels of each of those types of identified dental conditions. In at least one embodiment, dental health analyzers 834 additionally use information of multiple different types of identified dental conditions and/or associated severity levels to determine correlations and/or cause and effect relationships between two or more of the identified dental conditions. Multiple dental conditions may be caused by the same underlying root cause. Additionally, some dental conditions may serve as an underlying root cause for other dental conditions. Treatment of the underlying root cause dental conditions may mitigate or halt further development of other dental conditions. For example, malocclusion (e.g., tooth crowding and/or tooth spacing or gaps), tooth wear and caries may all be identified for the same tooth or set of teeth. Dental diagnostics hub 830 may analyze these identified dental conditions that have a common, overlapping or adjacent area of interest, and determine a correlation or causal link between one or more of the dental conditions. In example, dental diagnostics hub 830 may determine that the caries and tooth wear for a particular group of teeth is caused by tooth crowding for that group of teeth. By performing orthodontic treatment for that group of teeth, the malocclusion may be corrected, which may prevent or reduce further caries progression and/or tooth wear for that group of teeth. In another example, plaque, tooth staining, and gum recession may be identified for a region of a dental arch. The tooth staining and gum recession may be symptoms of excessive plaque. The dental diagnostics hub 830 may determine that the plaque is an underlying cause for the tooth staining and/or gum recession.

In at least one embodiment, currently identified dental conditions may be used by the dental diagnostics hub 830 to predict future dental conditions that are not presently indicated. For example, a heavy occlusal contact may be assessed to predict tooth wear and/or a tooth crack in an area associated with the heavy occlusal contact. Such analysis may be performed by inputting intraoral data (e.g., current intraoral data and/or past intraoral data) and/or the dental conditions identified from the intraoral data into a trained machine learning model that has been trained to predict future dental conditions based on current dental conditions and/or current dentition (e.g., current 3D surfaces of dental arches). The machine learning model may be any of the types of machine learning models discussed elsewhere herein. The machine learning model may output a probability map indicating predicted locations of dental conditions and/or types of dental conditions. Alternatively, the machine learning model may output a prediction of one or more future dental conditions without identifying where those dental conditions are predicted to be located.

Dental condition assessment tools 836 may enable doctors to view and perform assessments of various types of dental conditions. Each type of dental condition may be associated with its own unique dental condition assessment tool or set of dental condition assessment tools.

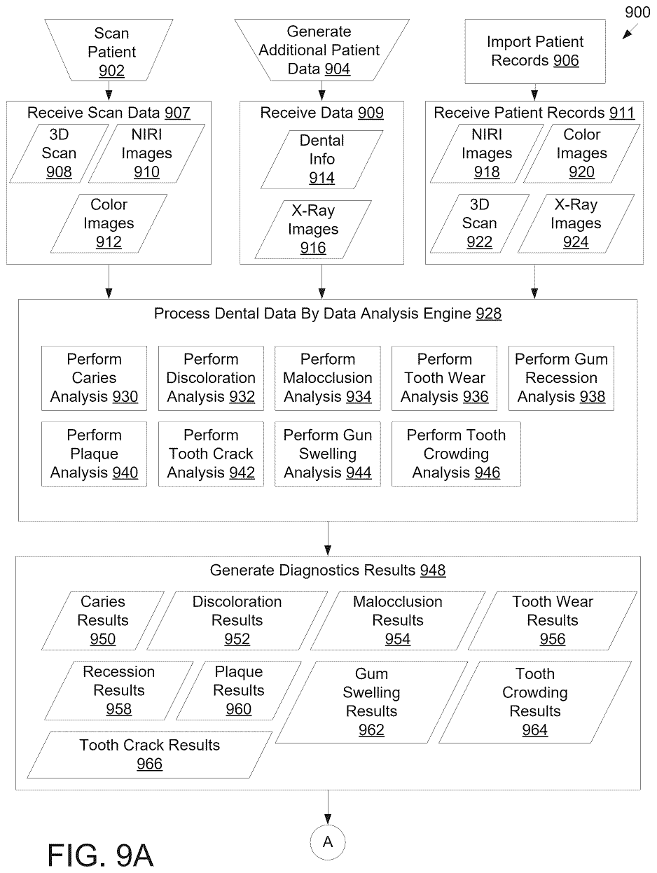

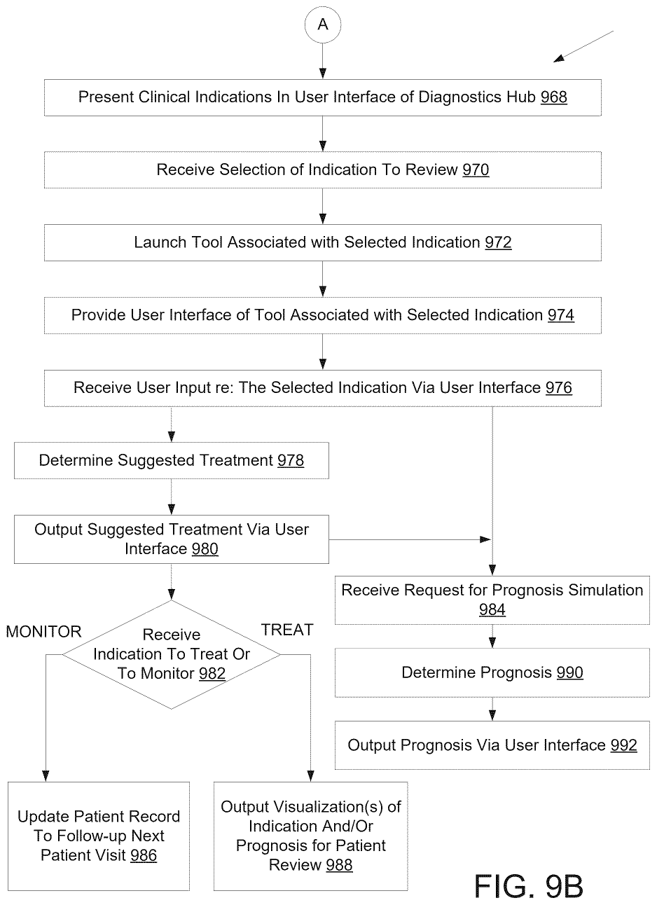

FIGS. 9A-9B illustrate a flow diagram for a method 900 of analyzing a patient's teeth and gums, in accordance with embodiments of the present disclosure. The method 900 may be performed by processing logic that comprises hardware (e.g., circuitry, dedicated logic, programmable logic, microcode, etc.), software (such as instructions run on a processing device), or a combination thereof. In one embodiment, processing logic corresponds to computing device 805 of FIG. 8 (e.g., to a computing device 805 executing an intraoral scan application 808 and/or a dental diagnostics hub 830).

At block 902, a doctor or dental practitioner scans a patient's intraoral cavity. At block 907, processing logic receives the scan data from the scan performed at block 902. The scan data may include 3D scan data (e.g., color or monochrome 3D scan data 908, which may be received in the form of point clouds, height maps, images, or other data types) and/or one or more 3D models of the patient's dental arches generated based on the scan data. The scan data may further include NIRI images 910 and/or color images 912. The 3D scan data 908, NIRI images 910 and/or color images 912 may all be generated by an intraoral scanner. At block 904, a doctor or dental practitioner may generate additional patient data. At block 909, processing logic receives the additional patient data. The additional patient data may include x-ray images 916 (e.g., bitewing x-ray images and/or panoramic x-ray images) and/or dental information 914 (e.g., such as observations of the dental practitioner, biopsy results, CBCT scan data, ultrasound data, etc.). At block 906, processing logic may import patient records for the patient being scanned. At block 911, processing logic may receive the imported patient records. The imported patient records may include historical patient data such as historical NIRI images 918, color images 920, 3D scan data or 3D models generated from such 3D scan data 922, x-ray images 924 and/or other information.

At block 928, processing logic processes the received dental data (including the data received at blocks 907, 909 and/or 911) using one or more data analysis engine (e.g., dental health analyzer(s) 834 of FIG. 8) to identify the presence of one or more types of dental conditions and/or severity levels for the one or more detected dental conditions. Processing logic may perform a caries analysis 930, a discoloration analysis 932, a malocclusion analysis 934, a tooth wear analysis 936, a gum recession analysis 938, a plaque analysis 940, a tooth crack analysis 942, a gum swelling analysis 944 and/or a tooth crowding analysis 946 (and/or tooth spacing analysis), as discussed herein above. The various analyses may include point-in-time analyses as well as time-dependent analyses that are based on data from multiple different times. For example, older scan data may be compared to recent scan data to determine whether dental conditions have improved, stayed the same, worsened, etc. Additionally, trajectories for the various dental conditions at various areas of interest where the dental conditions were identified may be determined and projected into the future and/or past. In at least one embodiment the outputs of one or more dental health analyzers are used (alone or together with intraoral data) as inputs to other dental health analyzers. For example, the presence or absence of malocclusion may be used as in input into a dental health analyzer that performs the caries analysis 930 and/or the dental health analyzer that performs plaque analysis 940.

At block 948, processing logic generates diagnostics results based on an outcome of the dental condition analyses performed at block 928. Processing logic may generate caries results 950, discoloration results 952, malocclusion results 954, tooth wear results 956, gum recession results 958, plaque results 960, gum swelling results 962, tooth crowding and/or spacing results 964 and/or tooth crack results 966. The diagnostics results may include detected AOIs associated with each of the types of dental conditions, and severity levels of the dental conditions for the AOIs. The diagnostics results may include qualitative measurements, such as size of an AOI, an amount of recession for a gum region, an amount of wear for a tooth region, and amount of change (e.g., for a caries, tooth wear, gum swelling, gum recession, tooth discoloration, etc.), a rate of change (e.g., for a caries, tooth wear, gum swelling, gum recession, tooth discoloration, etc.), and so on. The diagnostics results may further include qualitative results, such as indications as to whether a dental condition at an AOI has improved, has stayed the same, or has worsened, indications as to the rapidity with which the dental condition has improved or worsened, an acceleration in the improvement or worsening of the dental condition, and so on. An expected rate of change may have been determined (e.g., automatically or with doctor input), and the measured rate of change for a dental condition at an AOI may be compared to the expected rate of change. Differences between the expected rate of change and the measured rate of change may be recorded and included in the diagnostics results. Each of the diagnostics results may be automatically assigned a code on dental procedures and nomenclature (CDT) code or other procedural code for health and adjunctive services provided in dentistry. Each of the diagnostics results may automatically be assigned an appropriate insurance code and related financial information.

In at least one embodiment, image documentation of one or more identified procedures may be exported from scan data (e.g., intraoral images) and associated with specific treatment codes (e.g., CDT codes) for justification of a procedure performed or to be performed, such as for insurance coverage purposes. In an example, images of gingival inflammation and/or calculus (supragingival or subgingival if detectable) can go with (or be user configured to be attached to) periodontal procedure codes. In another example, images of caries can go with or be attached to restorative codes, images of fractures or abfractions can go with or be attached to occlusion and prosthodontic codes, images of crowding/spacing can go with or be attached to orthodontic codes, and so on. This may satisfy the requirements of some insurance companies, which may require documentation prior to authorizing certain procedures.

At block 968, processing logic presents clinical indications of the dental condition analysis results in a user interface of a dental diagnostics hub, such as shown in FIG. 1C. The clinical indications may additionally be automatically added to a patient chart. For example, a patient chart may automatically be updated to identify each identified dental condition, a tooth and/or gum region affected by the dental condition, a severity level of the dental condition, and/or other information about an AOI at which the dental condition was identified. The doctor may add notes about the dental conditions as well, which may also be added to the patient chart.

The information presented in the user interface may include qualitative results and/or quantitative results of the various analyses. In at least one embodiment, a dental diagnostics summary is shown that includes high level results, but that does not include low level details or detailed information underlying the high level results. All of the results of the analyses may be presented together in a unified view that improves clinical efficiency and provides for improved communication between the doctor and patient about the patient's oral health and how best to treat dental conditions. The summary of the dental condition results may not include or display specifics on where AOIs associated with particular dental conditions were identified and/or how many such AOIs were identified. Instead, in embodiments a minimum amount of information that is necessary to enable a doctor to formulate an initial impression about the patient's oral health may be presented. The dental conditions may be ranked based on severity level in embodiments. In at least one embodiment, the dental conditions are grouped into multiple classifications, where one classification may indicate that no issues were found (indicating that there is no need for the doctor to review those dental conditions), one classification may indicate that potential issues were found (indicating that the doctor might want to review those dental conditions, but that such review is not urgent), and/or one classification may indicate that issues were found (indicating that the doctor is recommended to immediately review those dental conditions).

The information presented in the user interface may include information identifying one or more new dental conditions that were detected in the current or most recent patient visit but not in the prior patient visit. The information presented in the user interface may include information identifying one or more preexisting dental conditions that have improved between the prior patient visit and the current or most recent patient visit. The information presented in the user interface may include information identifying one or more preexisting dental conditions that have worsened between the prior patient visit and the current or most recent patient visit. The information presented in the user interface may include information identifying one or more preexisting dental conditions that have not changed between the prior patient visit and the current or most recent patient visit.

At block 970, processing logic receives a selection of an indication to review. For example, a doctor may select caries indications to review. At block 972, processing logic launches one or more tools associated with the selected indication. At block 974, processing logic provides a user interface for the tool or tools associated with the selected indication, as shown with examples in FIGS. 2-6. Via the user interface, the doctor may review detailed information about the type of dental condition that was selected.

At block 976, processing logic may receive user input (e.g., from the doctor) regarding a selected indication via the user interface. The user input may include a user input defining one or more case specific areas of interest and/or issues (e.g., dental conditions) of interest for follow-up in future scans. In a future patient visit, the dentist may generate new intraoral data for the patient, and that new intraoral data may be used along with the definition of the AOIs and/or issues of interest when performing future analysis of the patient's dental health. The user input may additionally or alternatively include a user input defining customization for AOIs and/or issues (e.g., dental conditions) of interest for all patients. For example, the doctor may define criteria (e.g., thresholds) for detecting dental conditions and/or for assessing the severity of dental conditions. The doctor may additionally or alternatively override analysis results, such as by manually updating an AOI that was indicated as being an issue for a particular class of dental condition so that it is not labeled as an issue. The dental diagnostics hub may be customized for and/or by a doctor to enable that doctor to develop their own workflows to help walk a patient through their oral health, detected dental conditions, and options for addressing the dental conditions.

At block 978, processing logic determines a suggested treatment. Each of the types of dental conditions may be associated with one or more standard treatments that are performed in dentistry and/or orthodontics to treat that type of dental condition. Based on the locations of identified AOIs, the dental conditions for the identified AOIs, the number of AOIs having dental conditions and/or the severity levels of the dental conditions, a treatment plan may be suggested. A doctor may review the treatment plan and/or adjust the treatment plan based on their practice and/or preferences. In at least one embodiment the doctor may customize the dental diagnostics hub to give preference to some types of treatment options over other types of treatment options based on the doctor's preferences. Treatments may be determined for each of the identified dental conditions that are determined to have clinical significance.

At block 980, processing logic may output the suggested treatment or treatments via the user interface. In at least one embodiment, at block 984 processing logic may receive a request for a prognosis simulation. At block 990, processing logic simulates a prognosis of the dental condition with and/or without treatment. The prognosis simulation may be based on the determined AOIs and a selected dental condition. If a treatment was selected and/or suggested at block 980, then the suggested and/or selected treatment option may be used in determining the prognosis. In at least one embodiment, a first prognosis without treatment may be generated to show a likely course of the dental condition without treatment and a second prognosis with treatment may be generated to show a likely course of the dental condition with treatment. At block 992, the generated prognosis (or multiple prognoses) are output via the user interface. The prognosis or prognoses may be shown to a patient and/or may be sent to the patient for consideration (e.g., a link to the prognosis may be sent to the computing device of the patient).

The prognosis may include automatically generated patient communication data (also referred to as educational information) that facilitates the doctor communicating with the patient about the prognosis and possible treatments. Patient communication data may be generated for each of the types of detected dental conditions, and may be presented to the patient together via a unified presentation or separately as discrete presentations for one or more types of dental conditions. The patient communication data may include textual and/or graphical information explaining and highlighting the findings and prognoses in an easy way to understand for non-clinicians. The patient communication data may show prognoses of the patient using the patient's own dentition, projected into the future with and/or without treatment. The patient communication data may include data for one or a number of selected AOIs and/or dental conditions, or may include data for each of the AOIs and/or dental conditions or for each of the AOIs and/or dental conditions that exceed a particular severity level threshold or thresholds. In at least one embodiment, the patient wears an augmented reality (AR) or virtual reality (VR) display, and the findings and/or prognosis are shown via augmented reality and/or virtual reality.

In an example, the patient communication data may include some or all of the dental conditions that were identified that the doctor agreed should be addressed and/or monitored. The patient communication data may further include a comparison to dental conditions and/or AOIs of the patient at prior visits, and indications of how the AOIs and/or dental conditions have changed between visits. The patient communication data may include indications as to whether an AOI and/or dental condition was discussed previously, and a decision that was made about the AOI and/or dental condition. For example, the patent communication data may indicate that the patient already has been informed of a problem and that the doctor and/or patient are keeping an eye on the problem but are not planning on treating the problem at the present time.

Educational information may be presented, which may or may not be tailored based on the patient's dentition (e.g., using 3D models of the patient's dental arches). The educational information may show progression of the patient through different severity levels of a dental condition, using that patient's own dentition. In at least one embodiment, the dental condition analysis tools may be used to segment the patient's dental arches into teeth and gingiva, to identify dental conditions in the teeth and in the gingiva, and to predict and provide animations for progression of the various dental conditions for the patient's dental arches. Educational information may also show how the progression of dental conditions may be stopped or reversed with treatment options and/or with changes in patient behavior (e.g., brushing twice daily, flossing, wearing a night guard, etc.). Such educational information may be shown for each of the types of dental conditions discussed herein.

The information about dental conditions and/or AOIs to monitor, and the information about dental conditions and/or AOIs to treat, may be used to generate a customized report for the patient in an automated manner, with little or no doctor input. The patient communication data may further include sequencing information identifying first treatments to be performed and/or dental conditions to be addressed, subsequent treatments to be performed and/or dental conditions to be addressed, and so on. The patient communication data may indicate which treatments are optional and which treatments are necessary for the patient's dental health, and may further indicate urgency associated with each of dental conditions and associated treatments. For example, dental conditions that are emergencies may be identified, those dental conditions that should be addressed in the near future (e.g., next few months) may be identified, and those dental conditions that are not urgent but that should be addressed eventually may be identified. This enables the doctor and patient to prioritize treatment options. The patient communication data may further include information on the percentage of doctors that treat specific dental conditions, the types of treatments for those dental conditions that are performed and the rates at which those treatments are performed, and so on.

At block 982, processing logic receives an indication of one or more AOIs and/or dental conditions to monitor and/or to treat. For indications to treat an AOI and/or dental condition, the method proceeds to block 992. For indications to monitor an AOI and/or dental condition, the method proceeds to block 986.

At block 986, processing logic updates a patient record to follow-up regarding the AOIs and/or dental conditions that were identified for monitoring. At a next patient visit, processing logic will flag those AOIs and/or dental conditions that were marked for follow-up.

At block 992, processing logic may output visualizations of the indications and/or prognosis for patient review, as discussed above. The presented information may include information regarding insurance information (e.g., whether insurance covers a treatment) and/or cost. For example, the presented information may include a cost breakdown of the costs for each of the treatments to be performed to treat the one or more dental conditions. The patient may accept or decline one or more treatment options. Responsive to acceptance of a treatment option (or multiple treatment options), processing logic may automatically populate insurance paperwork with information about the dental condition(s) and/or treatment(s), and may automatically deliver the filled out insurance paperwork to an insurance company (e.g., via a discretionary portfolio management solution (DPMS) system) and/or obtain pre-authorizations from the insurance company (e.g., via a response received from the insurance company) prior to commencement of one or more treatments.

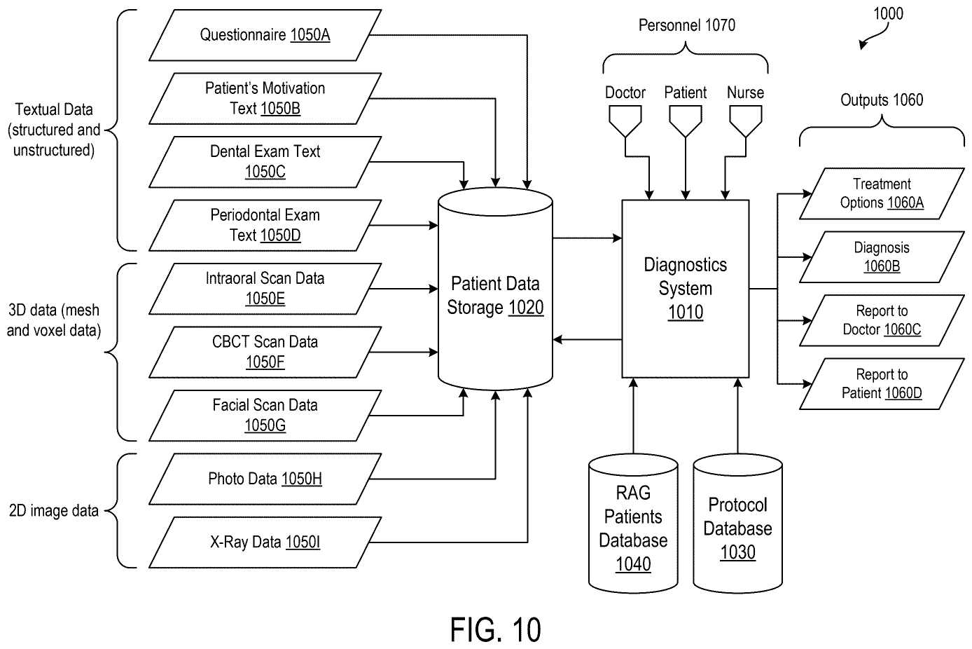

FIG. 10 illustrates an exemplary data pipeline 1000 for implementing a diagnostics system 1010 (e.g., a dental diagnostics system), in accordance with embodiments of the present disclosure. In at least one embodiment, the diagnostics system 1010 utilizes one or more language learning models to generate or update diagnostic records useful for determining treatment options, generating diagnoses, generating reports for doctors, generator reports for patients, other end uses. In at least one embodiment, the diagnostics system 1010 is implemented as part of the diagnostics hub according to any of the embodiments described herein. In at least one embodiment, one or more outputs 1060 from the diagnostics system 1010 may be presented visual in a GUI of a dental diagnostics hub according to any of the embodiments described herein.

Although the diagnostics system 1010 is depicted in FIG. 10 as a single device for ease of illustration, embodiments of the present disclosure are not limited to this representation. In at least one embodiment, the diagnostics system 1010 is implemented across a plurality of computing devices, which may be co-located or distributed across different geographic locations. In at least one embodiment, the diagnostics system 1010 is implemented in a cloud-based computing environment, such that one or more components of the diagnostics system 1010 are hosted on remote servers and accessed over a network (e.g., the Internet). In at least one embodiment, the diagnostics system 1010 is implemented as a hybrid system that includes both local computing resources (e.g., on-premises servers or workstations) and cloud-based computing resources. In at least one embodiment, the plurality of computing devices implementing the diagnostics system 1010 may include servers, workstations, edge computing devices, or any combination thereof. Accordingly, references herein to the diagnostics system 1010 should be understood to encompass any of these architectural configurations, whether centralized, distributed, cloud-based, or hybrid in nature.

In at least one embodiment, the diagnostics system 1010 implements one or more language models to generate one or more clinically-relevant outputs 1060. For example, each of the one or more language models may be a large language model (LLM). An LLM is a type of model designed to understand and generate human-like text, natural language, or the like. LLMs are generally built using dep learning techniques and trained on large datasets from diverse sources. LLMs often provide natural language understanding, text generation, contextual learning, instruction following based on nuanced or detailed prompts, and other functions. LLMs have advantages based on a large extent of knowledge mapped by the layers of the models, as well as an ability to make correct connections between concepts to generate relevant output. In at least one embodiment, an LLM implemented by the diagnostics system 1010 includes one or more deep neural networks that may incorporate transformer architectures, which allow the LLM to process and generate sequences of text by attending to different parts of input and output sequences. The LLM architecture may include multiple layers of neurons, each layer transforming the input data using weights and biases of the individual neurons that are learned during training. The LLM architecture may further include an attention mechanism that enables the LLM to weight the important of different words in a sentence relative to each other, allowing it to handle long-range dependencies in text. In at least one embodiment, the LLM includes speech-to-text processing logic (which may be considered a distinct component that is separate from the LLM or is implemented separately from the diagnostics system 1010). The speech-to-text processing logic may perform speech-to-text processing of input voice data.

In at least one embodiment, the diagnostics system 1010 is configured to process data in a variety of formats including, but not limited to: unstructured textual data (e.g., statements or sentences such as “tooth 17 has caries”), structured data (e.g., in JSON format, such as {“caries”: “17”}), embeddings or other types of latent space vector representations, or model data (e.g., 3D mesh data, volumetric data, various 2D image formats, etc.). The data may also be provided in its raw form or may be preprocessed. The data for a given patient may be collected from a variety of different sources and compiled into a patient record in patient data storage 1020, which may provide such data to the diagnostics system 1010 upon request. For data from difference sources, the textual data may be modified to identify the source of the data. For example, two different sets of data corresponding to the same physical measurement but are independently obtained from an intraoral scan and a CBTC scan may be stored as separate text strings or structured data along with an identifier or indication of the type of measurement that was performed to obtain it.

Examples of textual data in structured or unstructured form may include, but is not limited to, data in the form of a questionnaire 1050A, a patient's motivation 1050B, dental exam text 1050C, and periodontal exam text 1050D. The questionnaire 1050A may include a series of questions related to periodontal, biomechanical, dentofacial, and functional categories related to a patient, along with one or more sub-categories for which the doctor has indicated various risk levels, recommendations, notes pertaining to specific teeth, or other information. For example, in at least one embodiment, the questionnaire may provide at least a plurality of response options in the form of a list of selectable options that the doctor can choose, or allow the doctor to select from a rating scale. The patient's motivation text 1050B may correspond to, for example, a text transcription of a patient describing their reasons for seeking dental treatment, a survey filled out by a patient, or some other form of assessing the patient's motivation. The dental exam text 1050C and periodontal exam text 1050D may correspond, for example, to text written by a doctor describing the results of a dental exam and a periodontal exam, respectively.

In at least one embodiment, 3D data, such as mesh or volumetric (voxel) data, may include, but is not limited to, intraoral scan data 1050E, CBCT scan data 1050F, and facial scan data 1050G. In at least one embodiment, 2D image data may include, but is not limited to, photo data 1050H and x-ray data 1050I (e.g., panoramic x-ray data). In at least one embodiment, data extracted from 3D data 1050E-1050G and/or 2D image data 1050H-1050I may include, but is not limited to, distances between teeth, lengths of roots, distances between roots and bone surface, forward jaw surface information, backward jaw surface information, bone loss, bone density, and the like. In at least one embodiment, other types of input may be utilized by the diagnostics system 1010, including, but not limited to, parameters derived from articulation data and occlusion data. Each of the aforementioned parameters can be calculated and represented in textual form.

In addition to the various modalities discussed above, including data derived from scan data (e.g., intraoral scan data 1050E, CBCT scan data 1050F, facial scan data 1050G), 2D image data (e.g., photo data 1050H, x-ray data 1050I), and structured or unstructured textual data (e.g., questionnaire 1050A, patient's motivation text 1050B, dental exam text 1050C, periodontal exam text 1050D), the diagnostics system 1010 may receive one or more inputs directly as outputs from an artificial intelligence (AI) system or agent. For example, an upstream AI system or AI agent may perform preliminary analysis, feature extraction, data transformation, or other processing tasks, and provide the results of such processing as input to the diagnostics system 1010. In at least one embodiment, the AI system or agent may be a separate language model, a specialized machine learning model trained on domain-specific data, an autonomous agent configured to gather and preprocess patient information, or any combination thereof. In at least one embodiment, outputs from the AI system or agent may include, but are not limited to, extracted clinical parameters, preliminary diagnoses, risk assessments, treatment recommendations, summarized patient histories, or structured representations of unstructured data. In at least one embodiment, the diagnostics system 1010 is configured to receive and integrate such AI-generated outputs alongside data from other modalities. Accordingly, the diagnostics system 1010 may function as part of a larger ecosystem of AI systems and agents, where the output of one AI component serves as an input to another AI component in a coordinated processing pipeline.

In at least one embodiment, the diagnostics system 1010 has access to a protocol database 1030. The protocol database 1030 may include various sets of textual data descriptive written protocols, guidelines, preferences, or other practice-specific information associated with a doctor or a team of doctors. In at least one embodiment, the diagnostics system 1010 may determine and identify from data retrieved from the protocol database 1030 specific skillsets and/or preferences of a particular doctor (e.g., whether the doctor favors the use of implants, whether the doctor favors the use of veneers, tasks that the doctor prefers to delegate, etc.). In at least one embodiment, protocols, preferences, and other clinically-relevant information may be provided directly to the diagnostics system 1010 via personnel 1070 (e.g., a doctor, a patient, a nurse, etc.), for example, via a GUI. In at least one embodiment, the protocol database 1030 may be part of or directly under the control of the diagnostics system 1010, as illustrated in FIGS. 11-13.

In at least one embodiment, the diagnostics system 1010 has access to a retrieval augmented generation (RAG) patients database 1040. In at least one embodiment, the RAG patients database 1040 comprises data records describing patient data (including diagnoses, treatment options, etc.) for a set of prior patients. In at least one embodiment, the diagnostics system 1010 may provides a query to the RAG patients database 1040 based on parameters associated with a current patient whose data the diagnostics system 1010 is to process. The query is used to identify patient records in the RAG patients database 1040 that match or are similar to the parameters of the query, including demographic parameters, measurement parameters, Boolean parameters (e.g., presence/absence of jaw pain), or other clinically relevant parameters. In response, the RAG patients database 1040 provides a set of the identified patient records to the diagnostics system 1010, including, for example, information describing treatments that were performed for those patients and outcomes of the treatments. In at least one embodiment, the query may be based on one or more parameters specified by one or more personnel 1070 (e.g., a doctor, patient, nurse, etc.). In at least one embodiment, the query may be based on one or more parameters identified in or extracted from protocols or preferences stored in the protocol database 1030.

In at least one embodiment, outputs 1060 of the diagnostics system 1010 may include, but are not limited to, treatment options 1060A, a diagnosis 1060B, a report to a doctor 1060C, and a report to the patient 1060D. In at least one embodiment, one or more of the outputs may be generated as natural language text (e.g., in the form of a report). In at least one embodiment, the natural language text may be in the form of instructions for a downstream language model (e.g., an LLM that is configured to build treatment plans and recommendations based on textual instructions). In at least one embodiment, one or more of the outputs may be generated as structured data, and may be stored, for example, in the patient data storage 1020. In at least one embodiment, one or more of the outputs 1060 may be generated in the form of domain specific programming language code for use in one or more downstream processes or models (e.g., in information processing language code).

In at least one embodiment, outputs 1060 may be at least partially text-based expressed in natural language, and may include treatment information, digital practice management information and/or doctor-patient relationship information. The treatment information may be generated by the diagnostics system 1010 in conformance with preferences and protocols of the doctor utilizing the diagnostics system 1010 (e.g., based on data retrieved from the protocol database 1030). Treatment information may include information about a treatment (e.g., an orthodontic treatment, prosthodontic treatment, etc.) of the patient at hand, such as what operations are included in the treatment, a type of orthodontic appliance to use (e.g., braces, clear aligners, retainers, etc.), staging of the treatment, estimated treatment duration, specific treatment goals (e.g., alignment, bite correction, spacing), recommended interproximal reduction (IPR), tooth extractions and/or other pre-treatment or intra-treatment procedures, tooth attachments, amount of tooth movement at different stages, occlusal contact information, and so on. In the context of prosthodontics, treatment information might include a type of prosthesis (e.g., crown, bridge, dentures, implants, etc.), material choices (e.g., porcelain, metal, resin, etc.), step-by-step procedure details (e.g., tooth preparation, impressing taking, temporary prosthesis), coordination with other dental or medical treatments (e.g., periodontal therapy, surgery, etc.), and so on. Treatment information may additionally or alternatively include information on each procedure involved, including taking of impressions, x-rays, tooth preparation, appliance fittings, adjustments, frequency of visits, and/or whether sedation or anesthesia is to be used and how much. Treatment information may include expected outcomes such as aesthetic goals (e.g., expected changes in appearance, such as smile design, tooth alignment and facial symmetry), functional goals (e.g., improvement in bite, speech, chewing, and overall dental function), and/or any post-treatment care (e.g., including retainers, hygiene recommendations, follow-up visits, etc.). Treatment information may include a breakdown of costs, insurance coverage information, payment plans, etc. Treatment information may include pre-treatment patient instructions, post-treatment care, emergency protocols, etc. Treatment information may include before and after images, 3D models, radiographs, intraoral scans, etc. Treatment information may include progress notes such as detailed records of each patient visit including what was done, patient responses, and any modifications to the treatment plan.

In at least one embodiment, one or more of the outputs may be presented via a chat model, which may be accessible through a GUI. The chat model may provide the initial prompt, a chat history (e.g., back and forth between a user and the chat model), and/or treatment context information generated by the diagnostics system 1010. In at least one embodiment, the diagnostics system 1010 utilizes an instance of an LLM that was trained on customer data, medical services data, medical product data, patient historical data, etc. associated with a provider of medical services and/or medical products. In some embodiments, LLM is pretrained from knowledge of a particular medical domain. For information that is dynamically updated (e.g., because it is treatment specific, involves new data, etc.), a RAG implementation may be implemented.

FIGS. 11-13 illustrate variations of the data pipeline 1000 of FIG. 10, in accordance with embodiments of the present disclosure. FIG. 11 illustrates a data pipeline 1100 utilizing a text-based approach for providing input data to the diagnostics system 1010. For example, questionnaire 1050A, patient's motivation text 1050B, dental exam text 1050C, and periodontal exam text 1050D may be provided directly to the diagnostics system 1010 in structured or unstructured (e.g., natural language text) formats. In at least one embodiment, one or more pipelines may be utilized to convert scan data into structured or unstructured textual data. For example, an IO scan pipeline 1110 may be utilized to extract clinically-relevant data, parameters, or measurements from intraoral scan data 1050E and generated structured or unstructured textual data as input to the diagnostics system 1010. Similarly, a CBCT scan pipeline 1120 may be utilized to extract clinically-relevant data, parameters, or measurements from CBCT scan data 1050F, a facial scan pipeline 1130 may be utilized to extract clinically-relevant data, parameters, or measurements from facial scan data 1050G, a photo pipeline 1140 may be utilized to extract clinically-relevant data, parameters, or measurements from photo data 1050H, and an x-ray pipeline 1150 may be utilized to extract clinically-relevant data, parameters, or measurements from x-ray data 1050I.

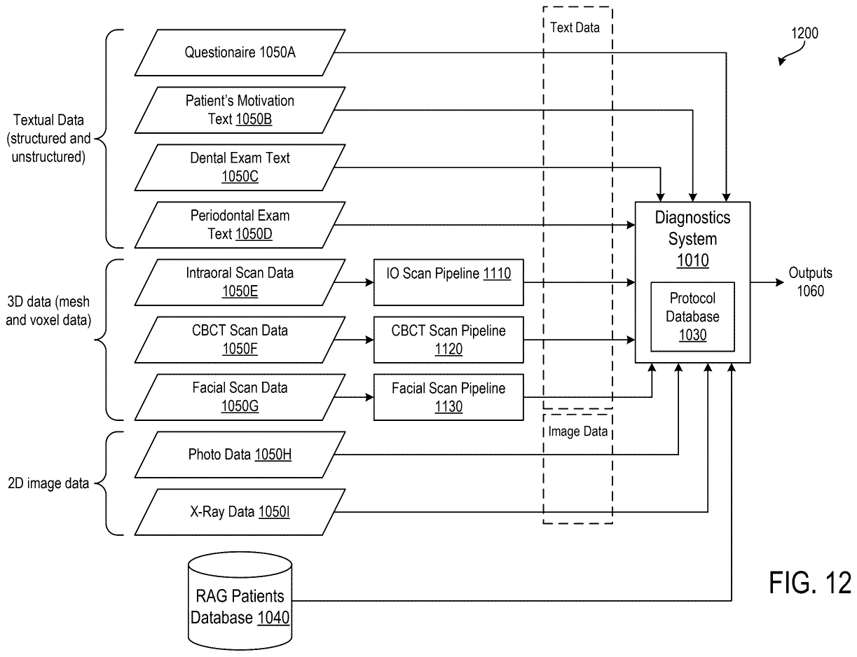

FIG. 12 illustrates a data pipeline 1200 utilizing both a text-based and an image-based approach for providing input data to the diagnostics system 1010. For example, intraoral scan data 1050E, CBCT scan data 1050F, and facial scan data 1050G may be processed by their respective pipelines, as discussed above with respect to FIG. 11. In at least one embodiment, the diagnostics system 1010 may received photo data 1050H and x-ray data 1050I directly without preprocessing or with minimal preprocessing. For example, the diagnostics system 1010 may include one or more trained machine learning models to extract clinically-relevant data, parameters, or measurements from the received photo data 1050H and/or x-ray data 1050I.

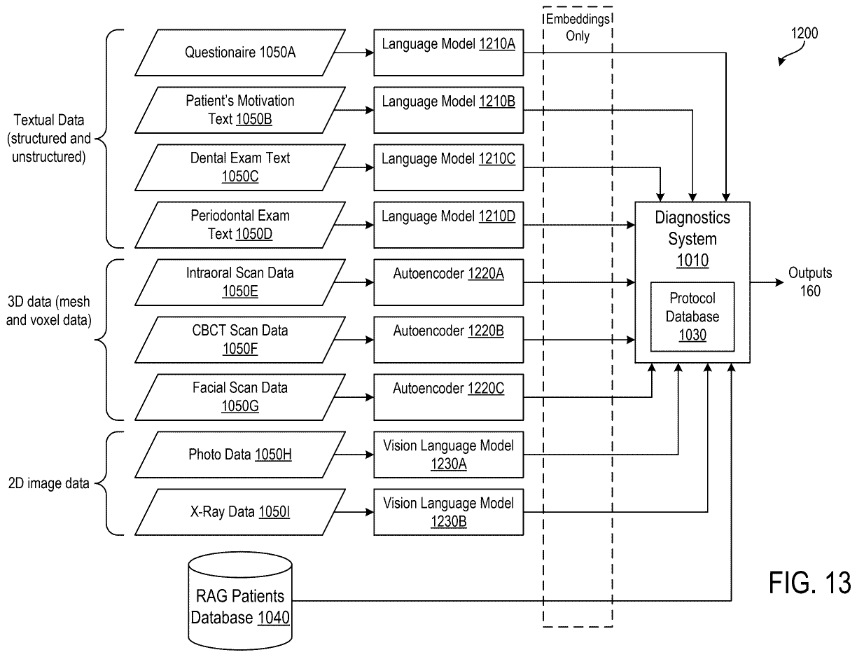

FIG. 13 illustrates a data pipeline 1300 utilizing an embeddings-based approach for providing input data to the diagnostics system 1010. In at least one embodiment, one or more language models 1210A-1210D (e.g., a large language model, such as GPT-4) may be used to generate embeddings from textual data 1050A-1050D, respectively. In at least one embodiment, the same language model (e.g., language model 1210A) may be used to generate the embeddings for all textual data 1050A-1050D.

In at least one embodiment, one or more autoencoders 1220A-1220C (e.g., a variational autoencoder) may be used to generate embeddings from 3D data 1050E-1050G, respectively. For example, each of the autoencoders 1220A-1220C may be trained to generate embeddings based on different types of 3D data inputs. In at least one embodiment, the same autoencoder (e.g., autoencoder 1220A) may be used to generate the embeddings for all 3D data 1050E-1050G.

In at least one embodiment, one or more vision language models 1230A-1230B (e.g., a multi-model system built on an LLM with a vision encoder, such as GPT-4V) may be used to generate embeddings from 2D image data 1050H-1050I, respectively. In at least one embodiment, the same vision language model (e.g., vision language model 1230A) may be used to generate the embeddings for all 2D image data 1050H-1050I.

Embeddings are dense vector representations of data (e.g., words, phrases, sentences, or paragraphs) that capture the semantic meaning of the data. Embeddings allow for a language model to process and understand relationships, context, and meaning within language. An LLM can learn an embedding via a training process optimized to predict or generate text, with the embeddings being adjusted through backpropagation. A typical embedding vector may include hundreds or thousands of dimensions, each representing abstract features of the word or phrase. The use of embeddings as inputs to the diagnostics system 1010 can advantageously result in faster computations, more nuanced relationships between words and concepts, and flexibility that allows LLMs of the diagnostics system 1010 to better understand novel sentences or words in context.

In at least one embodiment, the language models 1210A-1210D, autoencoders 1220A-1220C, and vision language models 1230A-1230B may utilize a tokenizer to generate tokens from preprocessed text. Each may further utilize an embedding generator to generate the embeddings from the generated tokens. For example, embeddings may be processed by one or more neural network layers of the models. During training, an LLM may process vast amounts of text data and learn the statistical relationships between words, phrases, sentences, and even larger contexts. The LLM predicts the next word or sequence of words based on the input it receives. This prediction is probabilistic, meaning the model calculates the likelihood of different possible continuations and selects the one with the highest probability. The model processes input as tokens (which may be words, subwords, or characters) and predicts the next token in the sequence by calculating the probability distribution over the possible tokens that could follow, given the preceding context.

It is to be understood that the variations depicted in FIGS. 11-13 are merely illustrative, and different sets of data may be provided to the diagnostics system 1010 in various combinations of text-based, image-based, and embeddings-based formats.

In at least one embodiment, the output 1060 may comprise an outcome record comprising one or more of a diagnosis, therapy options, exam recommendations, and a report for a doctor. In at least one embodiment, the diagnostics system 1010 may utilize a plurality of LLM agents trained to review, modify, supplement, and summarize the components of the outcome record according to different roles. For example, separate LLM agents may be trained to act as diagnosticians for the purposes of making, specifying, and explaining the diagnosis (e.g., re-formulate the diagnosis in more formal or more patient-friendly manner). In at least one embodiment, one or more agents may be trained to act as therapists to make, modify, and filter out recommendations for therapy options. In at least one embodiment, one or more agents may be trained to review exam recommendations and recommend various exams, including x-ray exams, CBCT exams, or the like. In at least one embodiment, one or more agents may be trained to review exam recommendations in view of redundant recommendations, and update the recommendations accordingly (e.g., removing a CBCT exam recommendation in view of a previously performed CBCT scan, or data duplicative of potential data that would be obtained by a new CBCT scan).

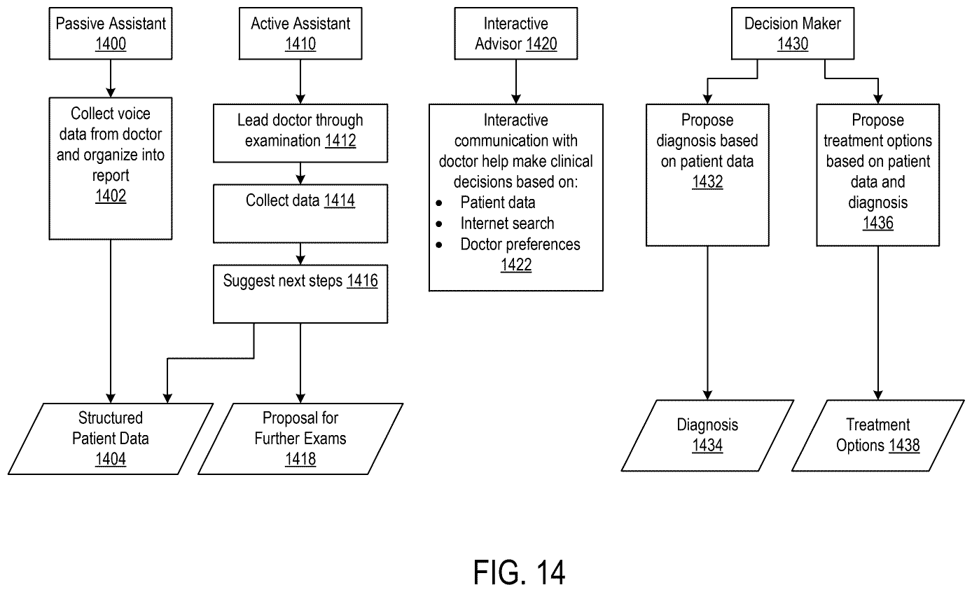

FIG. 14 includes flow diagrams illustrating potential implementations of the diagnostics system 1010 for processing and generating outputs, in accordance with embodiments of the present disclosure. For example, the diagnostics system 1010 may be adapted to act in various roles depending on the desired context of the doctor or team or doctors. For example, the roles may include, but are not limited to, a passive assistant 1400 role, an active assistant 1410 role, an interactive advisor 1420 role, and a decision maker 1430 role.

Block 1402 represents activities performed by the passive assistant 1400, which include, for example, collecting voice data from a doctor which is used for organizing into a report. The report may be generated as structured patient data 1404.

The active assistant 1410 may take on a more active role in direct the doctor's activities. For example, at block 1412, the doctor is led through examination via conversational outputs (in the form of visual text or a text-to-voice output). At block 1414, the active assistant collects data during the examination, for example, based on voice data collected from the doctor, from data directly input by the doctor (e.g., into a dental diagnostics hub), or a combination thereof. At block 1416, next steps may be suggested resulting in a report in the form of structured patient data 1404 or a proposal for further examinations 1418.

The interactive advisor 1420, at block 1422, may play an interactive role, for example, in the form of a chatbot interface or through a verbal communication with the doctor, providing the doctor with assistance in making clinical decisions based on collected patient data, Internet searches (e.g. databases such as PubMed, UpToDate, etc.), or based on doctor preferences known to the diagnostics system 1010.

The decision maker 1430 may be tasked with generating various proposals and recommendations, for example, based on a decision tree model. At block 1432, the decision maker 1430 may propose a diagnosis 1434 based on processed patient data. At block 1436, the decision maker 1430 may proposed treatment options 1438 based on patient data and a diagnosis (which may be the same diagnosis 1434 generated by the diagnostics system 1010 in its decision maker 1430 role).

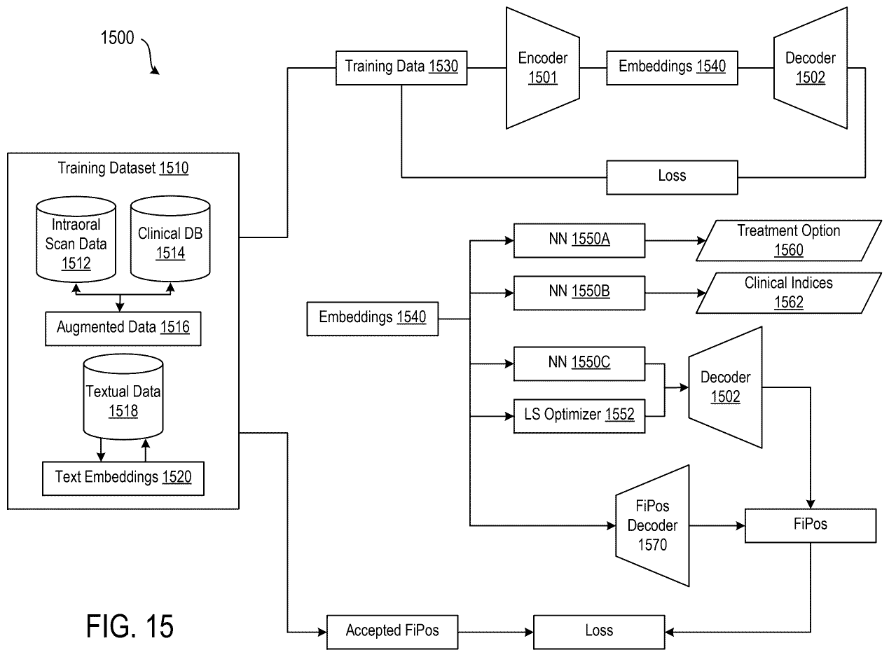

FIG. 15 illustrates training of an autoencoder 1500 used in the exemplary data pipeline 1000, in accordance with embodiments of the present disclosure. Data included in a training dataset 1510 for training the autoencoder 1500 may include intraoral scan data 1512 (which may be the same as intraoral scan data 1050E), data from a clinical database 1514 that includes records associated with a plurality of different patients, and augmented data 1516 corresponding to combinations, subsets, and/or post-processing of patient data from the intraoral scan data 1512 and the clinical database 1514. Textual data 1518 may include a repository of text-based (e.g., structured or unstructured) records pertaining to patients, including doctor's notes, patient questionnaires, etc. The textual data may have associated text embeddings 1520 derived from the textual data 1518.

In at least one embodiment, a subset of training data 1530 is provided to an encoder 1501 of the autoencoder 1500 to generate latent space embeddings 1540. The autoencoder 1500 may utilize a tokenizer to generate tokens from preprocessed text contained in the training data 1530. The encoder 1501 converts the tokens into embeddings by mapping each token into a dense vector of real numbers, which the model can then use for further computation. The autoencoder may include an embedding layer or embedding matrix that stores embeddings for all the tokens in the vocabulary. This matrix typically has a shape of (vocab_size, embedding_dim), where vocab_size is the total number of unique tokens in the vocabulary, and embedding_dim is the dimensionality of the embeddings (e.g., 128, 256, 512, etc.). For example, if the vocabulary has 10,000 tokens and the embedding size is 300, the embedding matrix would be of size (10,000×300). Each row in the embedding matrix corresponds to a token and contains its embedding vector. These vectors are typically dense, meaning that all the elements are non-zero, and they capture various semantic and syntactic properties of the token. When processing a token ID, the autoencoder 1500 performs a lookup operation in the embedding matrix to retrieve the corresponding embedding vector. The output of the embedding layer may be a matrix of embeddings corresponding to all the tokens in the input sequence. The generated embeddings 1540 and outputs generated by the decoder 1502 can be optimized with respect to the training data 1530 and corresponding text embeddings 1520.

Embeddings 1540 generated by the encoder 1501 may be utilized in connection with various neural networks 1550A-1550C of an LLM. The neural networks 1550A-1550C may include, for example, transformers, attention mechanisms, feed-forward networks, layer normalization, recurrent neural networks (RNNs), multi-layer perceptrons, and so on. Each layer refines and transforms the input embeddings based on learned weights and input context. The layers use these embeddings to perform tasks such as language modeling, text classification, and so on. After processing by each layer, the output may still be in the form of embeddings (e.g., dense vectors), which at this stage are enriched with contextual information. Accordingly, the embedding for a token now contains information not just about the token itself, but also about its relationship with other tokens in a sequence of tokens. The final output from the last neural network layer of the LLM is a set of contextualized embeddings. These embeddings are still in vector form and correspond to each token in the input sequence. However, they now represent the token in the context of the entire input sequence. These contextualized embeddings are typically passed through a linear layer (or a set of linear layers) to produce logits, which are raw scores for each token corresponding to the vocabulary size. The logits may then be processed by a softmax function to produce probabilities for each possible token in the vocabulary.

In at least one embodiment, a neural network 1550A may be trained to generate a treatment option 1560 from the embeddings. In at least one embodiment, a neural network 1550B may be trained to generate one or more clinical indices 1562. In at least one embodiment, a neural network 1550C may be used in combination with the decoder 1502 to optimize and transform embeddings into final position (“FiPos”) data for predicted treatment outcomes. FiPos data describes a target arrangement of the teeth (e.g., a desired and intended end result of orthodontic treatment) that can be received from a clinician in the form of a prescription, can be calculated from basic orthodontic principles, and/or can be extrapolated computationally from a clinical prescription. With a specification of the desired final positions of the teeth and a digital representation of the teeth themselves, the final position and surface geometry of each tooth can be specified to form a complete model of the tooth arrangement at the desired end of treatment. In at least one embodiment, the decoder 1502 computes positional changes to minimize loss in order to cause the generated FiPos data to be as close as possible to accepted FiPos data from the training dataset 1510. In at least one embodiment, latent space optimization of embeddings 1540 may be performed by a latent space optimizer 1552 in addition to or in lieu of using the neural network 1550C. In at least one embodiment, a FiPos decoder 1570 (which may be a neural network) may be trained to directly transform initial position data to final position data.

With the 3D model, a dental practitioner may determine a desired treatment outcome, which includes final positions and orientations for the patient's teeth. In at least one embodiment, the decoder 1502 or the FiPos decoder 1570 may be trained to generate a 3D model representative of a patient's dental arch (e.g., one or more meshes representative of individual teeth, meshes representative of gingiva, etc.) based on FiPos data. Processing logic of the diagnostics system 1010 may then determine a number of treatment stages to cause the teeth to progress from starting positions and orientations to the target final positions and orientations. In at least one embodiment, the generated 3D model may be utilized in a downstream pipeline, for example, to generate smile predictions.

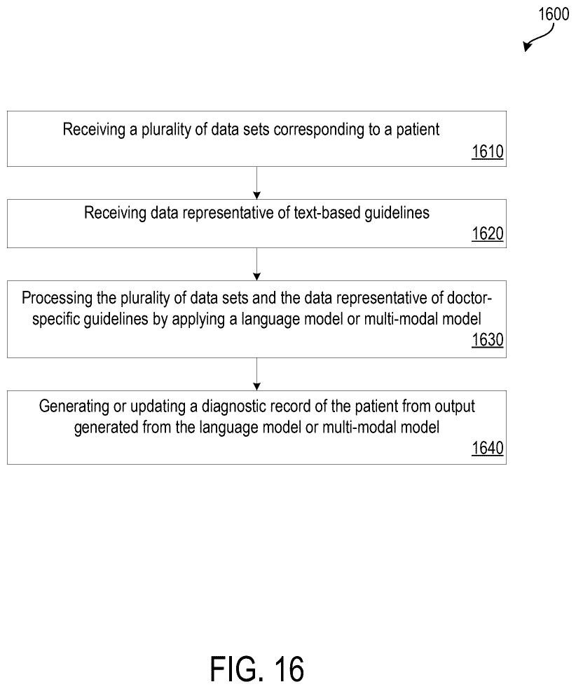

FIG. 16 illustrates a flow diagram for a method 1600 of generating or updating a diagnostic record of a patient, in accordance with embodiments of the present disclosure. The method 1600 may be performed by processing logic that comprises hardware (e.g., circuitry, dedicated logic, programmable logic, microcode, etc.), software (such as instructions run on a processing device), or a combination thereof. In one embodiment, processing logic corresponds to computing device 805 of FIG. 8 (e.g., to a computing device 805 executing an intraoral scan application 808 and/or a dental diagnostics hub 830).

At block 1610, processing logic (e.g., processing logic of the diagnostics system 1010) receives a plurality of data sets corresponding to a patient. In at least one embodiment, the plurality of data sets comprise one or more of textual data, 3D model data, or 2D image data. In at least one embodiment, the textual data comprises structured or unstructured text data selected from a questionnaire (e.g., questionnaire 1050A) such as a dental questionnaire, patient description data (e.g., patient's motivation text 1050B), dental exam data (e.g., dental exam text 1050C), periodontal exam data (e.g., periodontal exam text 1050D), or a combination thereof. In at least one embodiment, the 3D model data comprises intraoral scan data (e.g., intraoral scan data 1050E), CBCT scan data (CBCT scan data 1050F), facial scan data (facial scan data 1050G), other suitable 3D model data, or a combination thereof. In at least one embodiment, the 2D image data comprises one or more patient photos (e.g., photo data 1050H), one or more X-ray images (X-ray data 1050I), other suitable 2D image data, or a combination thereof.

In at least one embodiment, processing logic further generates a plurality of embeddings from one or more data sets of the plurality of data sets (e.g., utilizing one or more of language model 1210A-1210D, autoencoder 1220A-1220C, or vision language model 1230A-1230B). The generated embeddings may be received and processed by the diagnostics system in addition to or in lieu of the plurality of data sets from which the embeddings are derived. In at least one embodiment, each of the plurality of embeddings is computed by an LLM, an autoencoder, or a combination thereof. For example, in at least one embodiment, an autoencoder (e.g., a VAE) may be trained on embeddings data as training data so that the autoencoder can generate one or more clinically relevant outputs (e.g., when utilized by the diagnostics system 1010 for generating outputs 1060) or for processing data sets for use as input to the diagnostics system 1010. In at least one embodiment, the plurality of data sets are utilized to generate augmented data from intraoral scan data and clinical data descriptive of a corresponding patient as a training dataset for the diagnostics system.

In at least one embodiment, the clinically-relevant outputs may include one or more of a treatment option, clinical indices, a diagnosis, a medical report, or model data. In at least one embodiment, the autoencoder is configured to optimize embeddings data for generating one or more of a treatment option or clinical indices. In at least one embodiment, the autoencoder is configured to generate, based on embeddings data as input, final position data corresponding to a 3D model representative of a patient treatment outcome. In at least one embodiment, the autoencoder is utilized to perform latent space optimizations of embeddings.

At block 1620, processing logic receives data representative of text-based guidelines. In at least one embodiment, the text-based guidelines may comprise doctor-specific guidelines corresponding to a particular clinician or clinician's practice (e.g., data stored in the protocol database 1030). For example, the text-based guidelines may include various sets of textual data descriptive written protocols, guidelines, preferences, or other practice-specific information associated with a doctor or a team of doctors. In at least one embodiment, the guidelines may be expressed in conversational language. At block 1630, processing logic process the plurality of data sets and the data representative of text-based guidelines by applying a language model. In at least one embodiment, the language model comprises a large language model (LLM). In at least one embodiment, processing logic receives prior diagnosis data associated with the patient or other patients having similarities to the current patient. For example, processing logic may utilize one or more parameters of the current patient to provide as a query to a RAG patients database (e.g., RAG patients database 1040), from which the results are used by the processing logic in combination with data related to the current patient. In at least one embodiment, prior diagnosis data associated with the patient or other patients is processed by applying the language model thereto.

At block 1640, processing logic generates or updates a diagnostic record of the patient from output generated from the language model. In at least one embodiment, processing logic generates, from the diagnostic record, one or more of treatment or therapy options (e.g., treatment options 1060A), a diagnosis (e.g., diagnosis 1060B), a recommendation, or a report (e.g., report to doctor 1060C, report to patient 1060D). In at least one embodiment, a generated report may comprise a prognosis for one or more of a periodontal assessment, a biomechanical assessment, a functional assessment, or a dentofacial assessment.

In at least one embodiment, processing logic generates a diagnostic record comprising structured data. The structured data is derived at least partially from 3D model data or 2D image data within the plurality of data sets. In at least one embodiment, one or more physical measurements may be derived from the 3D model data or the 2D image data, and used to update the diagnostic record to include the derived physical measurements. In at least one embodiment, processing logic identifies measurements in the diagnostic record that are duplicative of or conflicting with one or more of the derived physical measurements. Processing logic may then reconcile the duplicative measurements in the diagnostic record based on the derived physical measurements. In at least one embodiment, a user of the diagnostics system 1010 may be prompted to select which data to keep or discard.

In at least one embodiment, the processing logic facilitates presentation of a user interface implementing chatbot functionality. The chatbot may be presented, for example, in a browser window for which a user may input a query. In at least one embodiment, the chatbot generates a textual response by utilizing data from the diagnostic record, for example, using an LLM to present the data in conversational language.

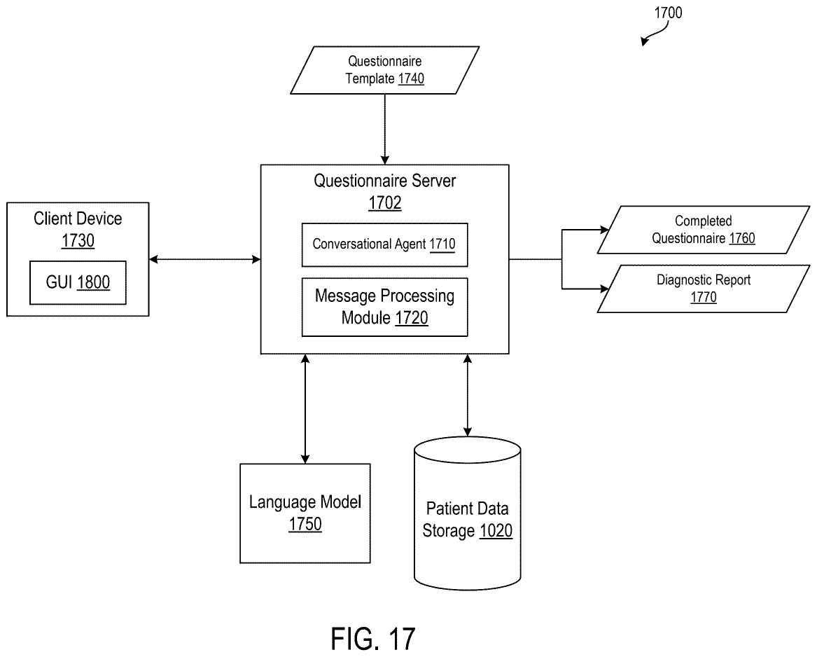

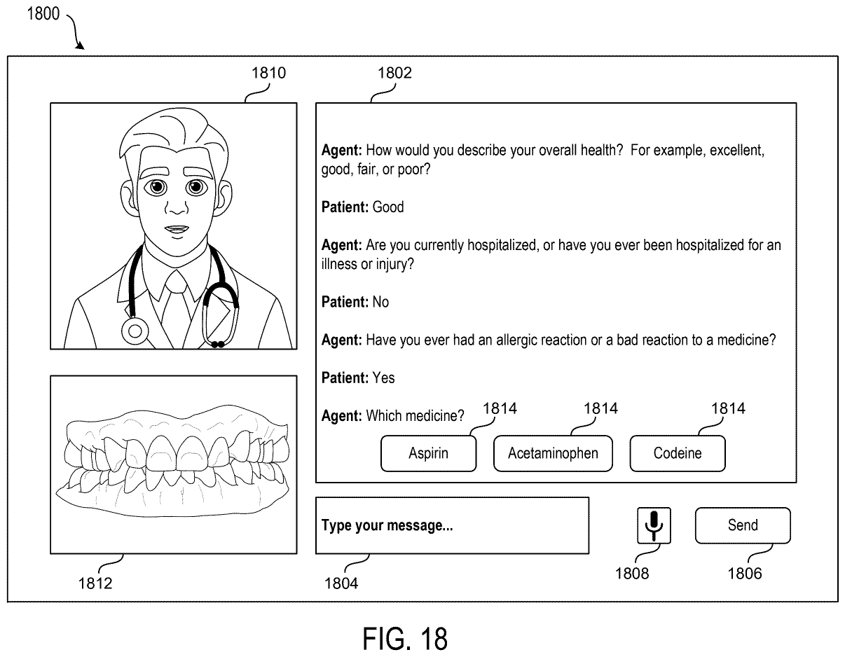

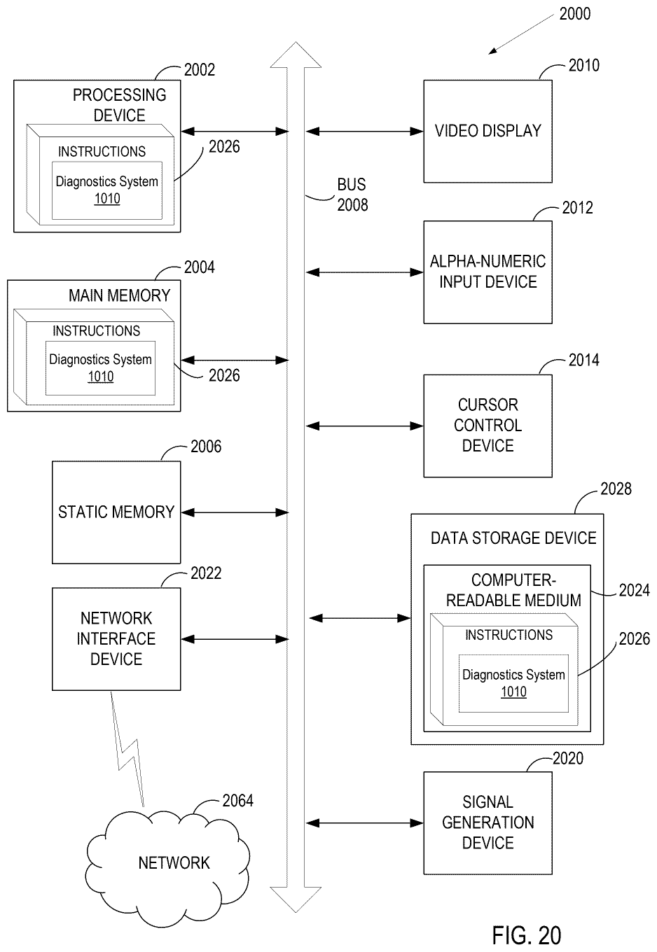

FIG. 17 illustrates an exemplary questionnaire system 1700 for providing an interactive conversational agent 1710 to converse with a patient, in accordance with embodiments of the present disclosure. In at least one embodiment, the questionnaire system 1700 includes a questionnaire server 1702 that hosts the conversational agent 1710 a message processing module 1720. The questionnaire server 1702 may be communicatively coupled to a client device 1730 via a network for implementing an interactive chat between a user (e.g., patient) and the conversational agent 1710. In at least one embodiment, the client device 1730 has functionality similar to the computing device 2000 described below with respect to FIG. 20, and may implement a GUI 1800 that allows a user of the device to converse with the conversational agent 1710. The GUI 1800 is described in greater detail with respect to FIG. 18.

In at least one embodiment, the conversational agent 1710 may utilize a language model 1750, which may be a large language model (LLM). In at least one embodiment, the LLM may be an LLM of the diagnostics system 1010, configured to process and interpret patient data, generate conversational responses, and facilitate interactive communication between the patient and the conversational agent 1710. The language model 1750 may be integrated with the questionnaire server 1702 and the message processing module 1720. In at least one embodiment, the conversational agent 1710 may be configured to access patient data stored in a patient data storage 1020. By retrieving relevant patient information, such as medical history, dental records, previous questionnaire responses, and other pertinent data, the conversational agent 1710 can generate patient-specific and context-sensitive messages tailored to the individual needs and circumstances of the user (e.g., patient) interacting via the client device 1730. The patient data storage 1020 may be securely connected to the questionnaire server 1702 and the message processing module 1720. In at least one embodiment, data utilized by the conversational agent 1710 may include or be derived from any of the data 1050A-1050I utilized by the diagnostics system 1010. In at least one embodiment, the data utilized by the conversational agent 1710 may include embeddings generated from such data utilizing any of the models 1210A-1210D, the autoencoders 1220A-1220C, or the models 1230A-1230B.

In at least one embodiment, the questionnaire server receives a questionnaire template 1740. In at least one embodiment, the conversational agent 1710 is trained on the questionnaire template 1740 so that it is able to guide the conversation with the patient toward obtaining information relevant to the specific fields and data points required for the questionnaire. In at least one embodiment, the conversational agent 1710 leverages the structure and content of the questionnaire template 1740 to dynamically generate questions, prompts, and follow-up inquiries that are designed to elicit medically-relevant responses from the patient. In at least one embodiment, the questionnaire template 1740 may include input fields such as patient name, date of birth, contact information, medical history, current dental symptoms, pain level, duration of symptoms, previous dental treatments, allergies, medications, insurance details, preferred appointment times, and others as would be appreciated by those of ordinary skill in the art. The conversational agent 1710, utilizing the message processing module 1720, may dynamically generate questions tailored to each of these input fields. For example, to elicit the patient's medical history, the conversational agent may ask, “Can you please describe any previous medical conditions or surgeries you have had?” For current dental symptoms, the agent may prompt, “Are you experiencing any pain or discomfort in your teeth or gums today?” These questions may be presented to the user via the GUI 1800 in at least one embodiment. The conversational agent 1710 may further adapt its inquiries based on the user's responses, providing follow-up questions or clarifications as needed to ensure completeness and accuracy of the data gathered.

In at least one embodiment, responsive to receiving responses from the client device 1730, the questionnaire server 1702 processes the incoming data through the message processing module 1720. In at least one embodiment, the message processing module 1720 is configured to parse, validate, and appropriately format each response according to the format of the questionnaire template 1740. The message processing module 1720 may perform data normalization, error checking, and mapping of user responses to corresponding fields within a questionnaire record. As each response is received, the module 1720 may update the questionnaire record in real time. Once the questionnaire record is determined to be sufficiently complete (e.g., all mandatory fields have been addressed and the data meets predefined validation criteria), the questionnaire server 1702 may generate a finalized version of the completed questionnaire 1760. In at least one embodiment, the questionnaire server 1702 may generate a diagnostic report 1770 based on the information provided in the completed questionnaire 1760, utilizing rule-based logic or a trained language model for analysis. The completed questionnaire 1760, and optionally the diagnostic report 1770, may then be transmitted to a storage device such as patient data storage 1020, or forwarded to the diagnostics system 1010 or a trained language model (e.g., language model 1210A) for further processing and review. For example, the completed questionnaire 1760 may correspond to the questionnaire 1050A as depicted in the data pipelines 1100 and 1200.

In at least one embodiment, the conversational agent 1710 enables both the questionnaire and its analysis to be edited by the doctor via a clinician-facing GUI interface, allowing for clarification of details during dental examinations or conversations with the patient. When the doctor uploads patient-specific documents into the conversational agent 1710, it can automatically extract information and respond to patient inquiries regarding that content. In at least one embodiment, the conversational agent 1710 performs partial analysis of the collected information before the doctor reviews it, presenting preliminary findings to the doctor prior to a consultation.

FIG. 18 illustrates an exemplary GUI 1800 to provide a patient access to the conversational agent (e.g., the conversational agent 1710), in accordance with embodiments of the present disclosure. As illustrated, the GUI 1800 includes a chat window 1802, a message field 1804, a send button 1806, an voice-to-text option 1808, a clinician avatar graphic 1810, and a medical graphic 1812. It is to be understood by those of ordinary skill in the art that additional components and functionality may be present, or that less than all of the components or functionality shown may be present. In addition, those of ordinary skill in the art would understand that the layout of the GUI 1800 may vary depending on the client device on which it is implemented and settings of the client device (such as accessibility settings). In various embodiments, the GUI 1800 may be presented as an in-browser interface accessible via standard web browsers, or as standalone software such as a dedicated application installed on a computing device. The GUI 1800 may be implemented on a range of client devices, including but not limited to personal computers (PCs), tablet computers, smartphones, and other mobile devices. Furthermore, in at least one embodiment, the GUI 1800 may be integrated with or implemented using an existing chat platform, such as WhatsApp or similar messaging services, to leverage existing infrastructure to facilitate patient access to the conversational agent.

In at least one embodiment, the user may interact with the conversational agent 1710 by entering text directly into a message field 1804, and transmitting their message by selecting a send button 1806. In at least one embodiment, the user may utilize a voice-to-text option 1808 that allows them to speak their responses, which may be converted into text and transmitted as a chat message. Additionally, the GUI 1800 may provide options for the user to have responses from the conversational agent 1710 read aloud, supporting accessibility and user preference for audio feedback. In at least one embodiment, the user may type or speak messages in a language that is different from what is presented in the 1802.

In at least one embodiment, the graphical user interface 1800 may include an avatar graphic 1810, which may visually represent a virtual character such as a doctor, dentist, or other medical personnel. The avatar graphic 1810 may be animated, providing dynamic facial expressions, gestures, and lip-syncing to enhance the realism and engagement of the interaction. In at least one embodiment, the responses generated by the conversational agent 1710 may be delivered as audio synchronized with the animated avatar graphic 1810 to create the appearance that the avatar is speaking directly to the user for a more personable and immersive experience. In addition to gathering information related to a questionnaire such as a dental questionnaire, the GUI 1800 may also enable users to perform a variety of other tasks. For example, users may be able to schedule appointments directly through the interface, request prescription refills, inquire about billing or insurance information, or receive reminders for upcoming visits or follow-up care. The system may further support requests for educational materials, such as information about dental procedures, oral hygiene tips, or post-treatment care instructions.

In at least one embodiment, the chat window 1802 may present the user with selectable options 1814 that allow the user to answer questions from the conversational agent 1710 without the need to type a response. These selectable options 1814 can be tailored to correspond to common answers or may be dynamically generated based on the user's medical history or previous responses. For example, when the conversational agent 1710 inquires about the type of medication the user is currently taking, the interface may display a list of medicines as selectable options 1814.

In at least one embodiment, the graphical user interface (GUI) 1800 may provide the user with options to upload various types of data, such as a photo of their face, a video capturing their face and/or teeth, a 3D face scan, or other relevant files. These upload options may be presented as selectable buttons or prompts within the GUI 1800. In at least one embodiment, the GUI 1800 may provide the user with the option to capture and upload a speech sample, such as a short audio recording. This functionality can be integrated as a selectable button or prompt within the chat window 1802. In at least one embodiment, the captured speech sample is transmitted to the questionnaire server 1702 and routed to a dedicated process within the pipeline configured to analyze speech characteristics, such as detecting potential speech defects or anomalies. The results of this speech analysis can be utilized by the questionnaire server 1702 to further personalize the interaction, generate relevant follow-up questions, or provide recommendations related to speech or oral health.

Upon receiving the uploaded data, the GUI 1800 may transmit it to the questionnaire server 1702, which is configured to route the data to a dedicated process or module within the processing pipeline. This separate process may perform analysis or extraction of pertinent information from the uploaded files, such as identifying dental features, facial symmetry, or other clinically relevant characteristics. In at least one embodiment, the results of these analyses are then communicated back to the message processing module 1720, which can utilize the extracted information to generate tailored responses, follow-up questions, or recommendations that are presented to the user via the GUI 1800.

In at least one embodiment, the system may further include a medical graphic 1812 that is displayed within the chat interface. The medical graphic 1812 may present dental-related data to the user, such as images or visualizations derived from scans, photographs, or other uploaded files. For example, if the user requests to view a scan of their teeth or facial structure, the medical graphic 1812 can render the relevant scan data directly in the chat window, allowing the user to visually inspect the information being discussed. In at least one embodiment, the medical graphic 1812 may serve as a visual aid to help the user better understand specific concepts or recommendations provided by the conversational agent 1710. For example, if the conversational agent is explaining a particular dental condition or treatment option, the medical graphic 1812 may display annotated diagrams, illustrations, or comparative images to clarify the explanation and enhance the user's comprehension. In situations where the conversational agent 1710 encounters difficulty in ascertaining a definitive answer required for the questionnaire, the conversational agent 1710 may further engage the user by asking clarifying questions. By leveraging partial analysis of the user's responses, the conversational agent can tailor its follow-up questions to resolve uncertainties and ensure the accuracy and completeness of the collected data.

In at least one embodiment, the patient may interact with the conversational agent 1710 via audio, in addition to or in lieu of interacting via the GUI 1800. For example, the patient may engage with the conversational agent 1710 through a voice-based interface, such as a telephone call, a voice-over-IP (VoIP) session, a smart speaker, or other audio-enabled device. In at least one embodiment, the conversational agent 1710 is configured to receive audio input from the patient, process the audio input using speech-to-text processing logic to generate textual data, and generate audio output using text-to-speech processing logic to deliver responses audibly to the patient. In at least one embodiment, the audio-based interaction may be conducted without requiring the patient to view or interact with a visual display to accomodate patients who have visual impairments, limited access to computing devices, or a preference for hands-free communication. In at least one embodiment, the conversational agent 1710 may conduct the audio-based interaction in real time, enabling a natural conversational flow similar to a telephone conversation with a human receptionist or medical professional. In at least one embodiment, the audio-based interaction may be asynchronous, allowing the patient to leave voice messages that are processed by the conversational agent 1710 and responded to at a later time. In at least one embodiment, the conversational agent 1710 may seamlessly transition between audio-based and GUI-based interactions within a single session, allowing the patient to switch modalities as needed.

The following is an illustrative use case of an audio-based interaction between a patient and the conversational agent 1710. A patient, upon receiving a reminder for an upcoming dental appointment, initiates a phone call to the dental practice. The call is answered by the conversational agent 1710, which greets the patient by name after identifying the patient based on the incoming phone number or a brief voice authentication. The conversational agent 1710 states: “Hello, this is your dental office. I see you have an appointment scheduled for next Tuesday at 2:00 PM. How may I assist you today?” The patient responds verbally: “I need to fill out my pre-appointment questionnaire.” The conversational agent 1710 proceeds to ask questions from a dental questionnaire in a conversational manner, such as: “Have you experienced any tooth pain or sensitivity since your last visit?” The patient responds: “Yes, I have been having some sensitivity on my lower left side when I drink cold beverages.” The conversational agent 1710 acknowledges the response and asks follow-up questions to gather additional details, such as: “How long have you been experiencing this sensitivity?” and “Does the sensitivity go away after a few seconds, or does it linger?” Once the questionnaire is complete, the conversational agent 1710 summarizes the collected information and confirms with the patient: “Thank you. I have noted that you are experiencing cold sensitivity on your lower left side that has been occurring for approximately two weeks. Is there anything else you would like to add before your appointment?” The patient confirms the summary, and the conversational agent 1710 stores the completed questionnaire in the patient's dental record for review by the dentist prior to the appointment.

In at least one embodiment, the conversational agent 1710 may be implemented in a virtual reality (VR) or augmented reality (AR) environment. In a VR implementation, the patient may wear a VR headset or other immersive display device that renders a fully virtual environment in which the patient interacts with the conversational agent 1710. For example, the VR environment may simulate a dental office waiting room or consultation room, within which a virtual avatar representing a dental professional (e.g., a dentist, hygienist, or receptionist) engages the patient in conversation. In an AR implementation, the patient may wear AR glasses, use an AR-enabled smartphone or tablet, or utilize another AR-capable device that overlays virtual elements onto the patient's real-world surroundings. For example, the AR environment may project a virtual avatar of a dental professional into the patient's physical space, such as their living room or kitchen, allowing the patient to interact with the conversational agent 1710 while remaining aware of their real-world environment. In at least one embodiment, the VR or AR implementation may include spatial audio, allowing the virtual avatar's voice to appear to originate from the avatar's location within the virtual or augmented space. In at least one embodiment, the patient may interact with the conversational agent 1710 in the VR or AR environment using voice commands, hand gestures, eye tracking, controller inputs, or any combination thereof. In at least one embodiment, the VR or AR environment may display visual aids, such as 3D models of teeth, dental anatomy diagrams, or representations of the patient's own dental scans, to enhance the patient's understanding of questions posed by the conversational agent 1710 or to illustrate dental conditions and treatment options.

The following is an illustrative use case of a patient interacting with the conversational agent 1710 in an AR environment. A patient at home receives a notification on their AR glasses indicating that their dental office has sent a pre-appointment questionnaire. The patient activates the notification, and an AR avatar representing a friendly dental assistant appears in front of the patient, rendered as a 3D holographic figure visible through the AR glasses. The avatar greets the patient: “Good afternoon! I'm here to help you complete your pre-appointment questionnaire before your visit next week. Do you have a few minutes to go through some questions?” The patient responds verbally: “Yes, I'm ready.” The avatar asks: “Have you noticed any changes in your teeth or gums since your last visit?” The patient responds: “I think one of my fillings might be loose.” The avatar displays a 3D model of a tooth with a filling hovering beside it and asks: “Can you point to or describe which tooth feels loose?” The patient gestures toward the upper right area of their mouth, and the avatar responds: “Thank you. I've noted that you have concerns about a filling in your upper right quadrant. The dentist will examine that area during your appointment.” The avatar continues with additional questions, displaying relevant visual aids as needed, until the questionnaire is complete. The avatar then summarizes the responses and confirms with the patient before storing the completed questionnaire in the patient's dental record.

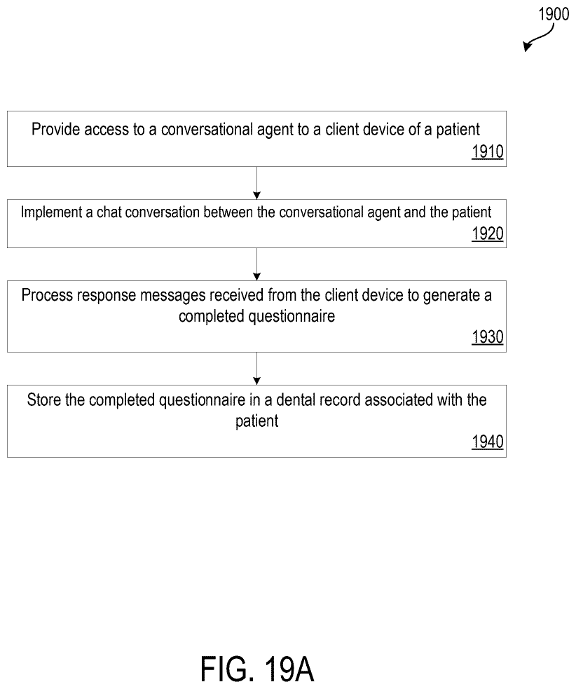

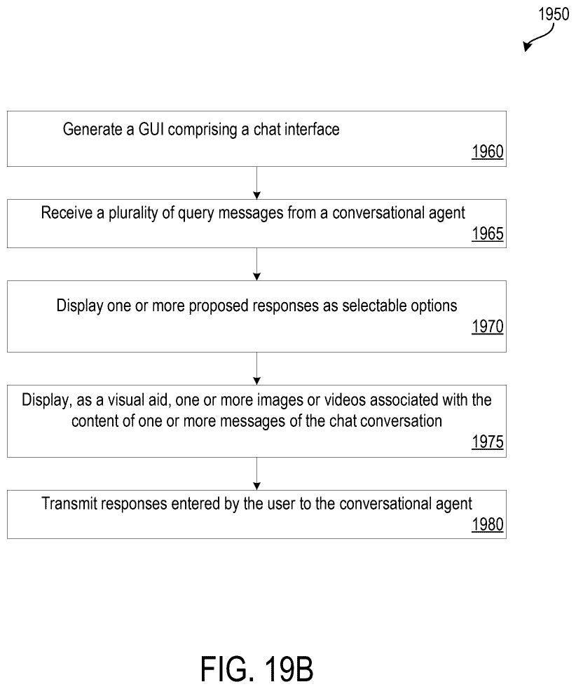

FIG. 19A illustrates a flow diagram for a method 1900 of providing a patient access to a conversational agent, in accordance with embodiments of the present disclosure. FIG. 19B illustrates a flow diagram for a method 1950 of providing a user interface on a client device for accessing a conversational agent, in accordance with embodiments of the present disclosure. The methods 1900 and 1950 may be performed by processing logic that comprises hardware (e.g., circuitry, dedicated logic, programmable logic, microcode, etc.), software (such as instructions run on a processing device), or a combination thereof. In one embodiment (e.g., of the method 1900), processing logic corresponds to the conversational agent 1710 of FIG. 17 (e.g., implemented by the questionnaire server 1702). In a further embodiment (e.g., of the method 1950), processing logic is implemented by the client device 1730.

Referring now to FIG. 19A, at block 1910, processing logic (e.g., processing logic of questionnaire server 1702) provides access to a conversational agent (e.g., the conversational agent 1710) to a client device (e.g., the client device 1730) of a patient. In at least one embodiment, the client device is configured to generate a GUI (e.g., the GUI 1800) that allows the patient to interact with the conversational agent, for example, through an exchange of text messages and/or through an audio conversation. In at least one embodiment, the conversational agent comprises an LLM (e.g., is integrated with an LLM or is communicatively coupled to an LLM, such as the language model 1750).

In at least one embodiment, the GUI is configured to present for display an avatar representative of a dental practitioner (e.g., via the avatar image 1810). In at least one embodiment, the GUI is configured to present for display, as a visual aid, one or more images or videos associated with the content of one or more of the plurality of messages (e.g., the medical graphic 1812).

In at least one embodiment, the GUI is configured to present for display, as a visual aid, text, one or more images, or one or more videos associated with content of one or more of the plurality of messages. In at least one embodiment, the text, one or more images, or one or more videos comprise clinical educational material configured to assist the patient in understanding clinical aspects of the chat conversation. In at least one embodiment, the clinical educational material is predefined and stored in a database. In at least one embodiment, the clinical educational material is generated dynamically based on a topic under discussion during the chat conversation. For example, if the patient mentions a medical condition, additional information related to that condition may be generated in real-time or near real-time and presented via the GUI. In at least one embodiment, a language of the chat conversation is changeable in response to a request from the patient.

In at least one embodiment, the one or more images or videos corresponds to scan data associated with the patient or a visual representation derived from the scan data. Such scan data may comprise one or more of intraoral scan data, CBCT data, facial scan data, or a combination thereof. The one or more images or videos corresponds to one or more patient photos or videos, one or more X-ray images, or a combination thereof. For example, in at least one embodiment, the one or more images or videos comprises pre-treatment and predicted post-treatment images or videos of the patient's face and/or dentition.

At block 1920, processing logic implements a chat conversation between the conversational agent and the patient. In at least one embodiment, the chat conversation may be text-based and take place in a chat window (e.g., chat window 1802), for which a plurality of messages are exchanged between the conversational agent and the patient. In at least one embodiment, the chat messages may include query messages transmitted to the client device (e.g., which may be derived from a questionnaire) and response messages received from the client device corresponding to the patient's responses to the query messages.

In at least one embodiment, the questionnaire is a medical questionnaire, which may include, but is not limited to, a general health questionnaire, a dental questionnaire, an oral health questionnaire, or any combination thereof. For example, a general health questionnaire may gather information about the patient's overall medical history, chronic conditions, current medications, allergies, family medical history, lifestyle factors (e.g., smoking, alcohol consumption, diet, exercise), and other health-related information that may be relevant to the patient's dental care or overall well-being. An oral health questionnaire may focus on conditions and symptoms related to the patient's mouth, teeth, gums, jaw, tongue, and related structures, which may overlap with but is not limited to traditional dental care. In at least one embodiment, the questionnaire need not be limited to health-related topics. For example, the questionnaire may include questions related to administrative matters, such as insurance information, billing preferences, appointment scheduling preferences, contact information updates, consent forms, or other non-clinical information relevant to the patient's relationship with the dental or medical practice. In at least one embodiment, the questionnaire may include questions related to patient satisfaction, feedback on prior visits, or preferences for future care. A dental questionnaire may focus on, for example, the patient's dental history, current symptoms (such as tooth sensitivity, pain, or bleeding gums), previous dental treatments, oral hygiene habits, dietary factors affecting dental health, any known allergies to dental materials or medications, history of jaw pain or temporomandibular joint (TMJ) disorders, bruxism or teeth grinding habits, use of tobacco or alcohol, presence of dental prosthetics or orthodontic appliances, frequency and recency of dental visits, and any concerns regarding cosmetic dental procedures.

In at least one embodiment, the conversational agent transmits data representative of one or more proposed responses to the client device. The client device may present for display via the GUI the one or more proposed responses as selectable options (e.g., selectable options 1814). In at least one embodiment, the one or more proposed responses is derived at least partially from a dental record of the patient or a previously received response message.

In at least one embodiment, one or more of the query messages are generated responsive to one or more of the response messages. In at least one embodiment, one or more of the query messages is derived at least partially on a dental record of the patient (e.g., a record from the patient storage 1020). In at least one embodiment, processing logic generates a contextual message in response to a response message received from the client device, and transmits the response message to the client device. In at least one embodiment, the contextual message generated in response to a response message may be utilized to clarify or elaborate on questions necessary for the completion of a dental questionnaire. For example, if the patient indicates a prior hospitalization, the contextual message requests additional details including a date of hospitalization, a reason for hospitalization, and whether a surgical procedure was performed. In at least one embodiment, the contextual message is generated to request clarification of additional information based on one or more clinical guidelines. In at least one embodiment, the contextual message is generated to support one or more conversational scenarios, comprising one or more of: clarification of operation of the chat conversation or completion of the medical questionnaire; clarification of medical terminology; modification or reversal of one or more previously provided responses; acknowledgment of a refusal to provide a response; or handling attempts to interfere with the questionnaire and returning the conversation to a primary workflow.

At block 1930, processing logic processes the response message received from the client device to generate a completed questionnaire (e.g., the completed questionnaire 1760). In at least one embodiment, processing logic includes textual data in the questionnaire that is derived at least partially from a previous dental questionnaire of the patient, patient description data, dental exam data, periodontal exam data, or a combination thereof. In at least one embodiment, processing logic inserts textual data derived from the processed response messages into text fields of the dental questionnaire. In at least one embodiment, the completed questionnaire is in the form of structured text data, unstructured text data, or a combination thereof.

At block 1940, processing logic stores the completed questionnaire in a dental record associated with the patient (e.g., in the patient data storage 1020). In at least one embodiment, processing logic transmits the completed questionnaire to a dental diagnostics system (e.g., the diagnostics system 1010).

Referring now to FIG. 19B, at block 1955, processing logic (e.g., processing logic of client device 1730) generates for display a GUI (e.g., the GUI 1800) comprising a chat interface to facilitate a chat conversation between a user (e.g., patient) of a client device and a conversational agent (e.g., the conversational agent 1710). In at least one embodiment, the GUI displays an avatar representative of a dental practitioner.

At block 1965, the GUI receives a plurality of query messages from the conversational agent pertaining to a dental health questionnaire. In at least one embodiment, one or more of the query messages are generated responsive to one or more of the response messages. In at least one embodiment, the query messages are presented to the user in text form or audio form via the GUI.

At block 1970, processing logic displays one or more proposed responses as selectable options (e.g., the selectable options 1814) in the GUI. In at least one embodiment, the one or more proposed responses is derived at least partially from a dental record of the user or a previously received response message.

At block 1975, processing logic displays, as a visual aid, text, one or more images, or one or more videos associated with the content of one or more messages of the chat conversation. In at least one embodiment, the one or more images or videos corresponds to scan data associated with the user or a visual representation derived from the scan data. In at least one embodiment, the scan data comprises one or more of intraoral scan data, CBCT data, facial scan data, or a combination thereof. In at least one embodiment, the one or more images or videos corresponds to one or more patient photos or videos, one or more X-ray images, or a combination thereof. In at least one embodiment, the one or more images or videos comprises pre-treatment and predicted post-treatment images or videos of the patient's face and/or dentition.

At block 1980, processing logic transmits responses entered by the user of the client device to the conversational agent.

FIG. 20 illustrates a diagrammatic representation of a machine in the example form of a computing device 2000 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein. In various embodiments, the computing device 2000 may correspond to one or more of the computing device 805 of FIG. 8, the questionnaire server 1702 of FIG. 17, or the client device 1730 of FIG. 17.

The example computing device 2000 includes a processing device 2002, a main memory 2004 (e.g., read-only memory (ROM), flash memory, dynamic random access memory (DRAM) such as synchronous DRAM (SDRAM), etc.), a static memory 2006 (e.g., flash memory, static random access memory (SRAM), etc.), and a secondary memory (e.g., a data storage device 2028), which communicate with each other via a bus 2008.

Processing device 2002 represents one or more general-purpose processors such as a microprocessor, central processing unit, or the like. More particularly, the processing device 2002 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 2002 may also be one or more special-purpose processing devices such as an application specific integrated circuit (ASIC), a field programmable gate array (FPGA), a digital signal processor (DSP), network processor, or the like. Processing device 2002 is configured to execute the processing logic (instructions 2026) for performing operations and steps discussed herein.

The computing device 2000 may further include a network interface device 2022 for communicating with a network 2064. The computing device 2000 also may include a video display unit 2010 (e.g., a liquid crystal display (LCD) or a cathode ray tube (CRT)), an alphanumeric input device 2012 (e.g., a keyboard), a cursor control device 2014 (e.g., a mouse), and a signal generation device 2020 (e.g., a speaker).

The data storage device 2028 may include a machine-readable storage medium (or more specifically a non-transitory computer-readable storage medium) 2024 on which is stored one or more sets of instructions 2026 embodying any one or more of the methodologies or functions described herein. A non-transitory storage medium refers to a storage medium other than a carrier wave. The instructions 2026 may also reside, completely or at least partially, within the main memory 2004 and/or within the processing device 2002 during execution thereof by the computer device 2000, the main memory 2004 and the processing device 2002 also constituting computer-readable storage media.

The computer-readable storage medium 2024 may also be used to store data for implementing the diagnostics system 1010, which may correspond to the similarly named component of FIG. 8, or may store data for implementing the questionnaire system 1700 of FIG. 17. The computer readable storage medium 2024 may also store a software library containing methods for the diagnostics system 1010. While the computer-readable storage medium 2024 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any non-transitory medium (e.g., a medium other than a carrier wave) that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

It is to be understood that the above description is intended to be illustrative, and not restrictive. Many other embodiments will be apparent upon reading and understanding the above description. Although embodiments of the present disclosure have been described with reference to specific example embodiments, it will be recognized that the disclosure is not limited to the embodiments described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

Claim language or other language herein reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

All references cited throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, are disclosed separately. When a Markush group or other grouping is used herein, all individual members of the group and all combinations and sub-combinations possible of the group are intended to be individually included in the disclosure.

It must be noted that as used herein and in the appended claims, the singular forms “a”, “an”, and “the” include plural reference unless the context clearly dictates otherwise. Thus, for example, reference to “a component” includes a plurality of such components and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. Nothing herein is to be construed as an admission that the invention is not entitled to antedate such disclosure by virtue of prior invention.

Whenever a range is given in the specification, for example, a temperature range, a time range, or a composition or concentration range, all intermediate ranges and subranges, as well as all individual values included in the ranges given are intended to be included in the disclosure. As used herein, ranges specifically include the values provided as endpoint values of the range. For example, a range of 1 to 100 specifically includes the end point values of 1 and 100. It will be understood that any subranges or individual values in a range or subrange that are included in the description herein can be excluded from the claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1-80. (canceled)

81. A method comprising:
receiving, by a diagnostics system, a plurality of data sets corresponding to a patient;
receiving, by the diagnostics system, data representative of text-based guidelines;
processing the plurality of data sets and the text-based guidelines by applying a language model of the diagnostics system, wherein the language model is configured to process one or more modalities; and
generating or updating a diagnostic record of the patient from output generated from the language model.

82. The method of claim 81, wherein the text-based guidelines comprise doctor-specific guidelines, and wherein the language model comprises a large language model (LLM).

83. The method of claim 81, wherein the plurality of data sets comprise one or more of textual data, 3D model data, or 2D image data.

84. The method of claim 81, further comprising:
receiving, by the diagnostics system, prior diagnosis data associated with the patient, wherein processing the plurality of data sets further comprises processing the prior diagnosis data by applying the language model.

85. The method of claim 81, further comprising:
generating a plurality of embeddings from the plurality of data sets, wherein each of the plurality of embeddings is computed by a large language model (LLM), an autoencoder, or a combination thereof; and
providing the embeddings as training data to the autoencoder to train the autoencoder to generate one or more of a treatment option, clinical indices, a diagnosis, a medical report, or model data.

86. The method of claim 81, further comprising:
generating, from the diagnostic record, one or more of a diagnosis, therapy option, recommendation, or report, wherein the report comprises a prognosis for one or more of a periodontal assessment, a biomechanical assessment, a functional assessment, or a dentofacial assessment.

87. The method of claim 81, further comprising:
presenting a user interface implementing a conversational agent;
receiving, as input to the conversational agent, an input query from a user; and
generating, as output from the conversational agent, a textual, verbal, or visual response based on the diagnostic record.

88. The method of claim 81, wherein the plurality of data sets comprise one or more of 3D model data or 2D image data, the method further comprising:
deriving one or more physical measurements from the 3D model data or the 2D image data;
updating the diagnostic record to include the one or more physical measurements;
identifying measurements in the diagnostic record that are duplicative of one or more of the derived physical measurements; and
reconciling the duplicative measurements in the diagnostic record based on the derived physical measurements.

89. A system comprising:
a memory; and
a processing device operatively connected to the memory, the processing device to perform the method of claim 81.

90. A computer readable medium comprising instructions that, when executed by a processing device, cause the processing device to perform the method of claim 81.

91. A method comprising:
receiving, by a diagnostics system, a plurality of data sets corresponding to a patient, wherein the plurality of data sets comprise data of different modalities;
receiving, by the diagnostics system, data representative of text-based guidelines;
processing, by the diagnostics system, each data set using a modality-specific machine learning model selected from a plurality of machine learning models to generate a representation of the data set, each modality-specific machine learning model being trained to process data of a specific modality;
aggregating the representations;
providing the aggregated representations and the text-based guidelines as input to a language model of the diagnostics system; and
generating or updating a diagnostic record of the patient based on output from the language model.

92. The method of claim 91, wherein each modality-specific machine learning model of the plurality of machine learning models is implemented by the diagnostics system or is utilized by the diagnostics system as an external or integrated resource.

93. The method of claim 92, wherein a first modality-specific machine learning model of the plurality of machine learning models comprises a language model that is trained to generate textual data or embeddings from structured or unstructured textual data.

94. The method of claim 92, wherein a second modality-specific machine learning model of the plurality of machine learning models comprises an autoencoder that is trained to generate textual data or embeddings from three-dimensional (3D) model data.

95. The method of claim 92, wherein a third modality-specific machine learning model of the plurality of machine learning models comprises a vision language model that is trained to generate textual data or embeddings from two-dimensional (2D) image data.

96. The method of claim 91, further comprising:
receiving embeddings data generated from one or more sets of data corresponding to patient data; and
providing the embeddings data as training data to each of the plurality of modality-specific machine learning models.

97. A system comprising:
a memory; and
a processing device operatively connected to the memory, the processing device to perform the method of claim 91.

98. A computer readable medium comprising instructions that, when executed by a processing device, cause the processing device to perform the method of claim 91.

99. A method comprising:
providing, to a client device of a patient, access to a conversational agent, wherein the client device is to generate a graphical user interface (GUI) that allows the patient to interact with the conversational agent;
implementing a chat conversation between the conversational agent and the patient, the chat conversation comprising a plurality of messages exchanged between the conversational agent and the patient, wherein the plurality of messages comprises:
query messages transmitted to the client device derived from a medical questionnaire; and
response messages received from the client device corresponding to responses of the patient to the query messages;
processing the response messages received from the client device to generate a completed questionnaire; and
storing the completed questionnaire in a dental record associated with the patient.

100. The method of claim 99, wherein the conversational agent comprises a large language model (LLM), and wherein the GUI is configured to present for display an avatar representative of a dental practitioner.
